(19) United States
(12) Patent Application Publication
(10) Pub. No.: US 2025/0189428 A1
(43) Pub. Date: Jun. 12, 2025

(54) COMPOSITIONS AND METHODS FOR SINGLE WELL MULTIPLEXED CALIBRATION AND COMPENSATION

(71) Applicant: Slingshot Biosciences, Inc., Emeryville, CA (US)

(72) Inventors: Jeffrey KIM, Berkeley, CA (US); Anh Tuan NGUYEN, San Francisco, CA (US)

(73) Assignee: Slingshot Biosciences, Inc., Emeryville, CA (US)

(21) Appl. No.: 19/057,384

(22) Filed: Feb. 19, 2025

Related U.S. Application Data
(63) Continuation of application No. PCT/US2023/072659, filed on Aug. 22, 2023.
(60) Provisional application No. 63/400,039, filed on Aug. 22, 2022.

(51) Int. Cl. G01N 15/10; G01N 15/14

(52) U.S. Cl. CPC G01N 15/1012; G01N 15/147; G01N 2015/1006

(57) ABSTRACT

The present disclosure relates to compositions comprising a plurality of modified particles that allow for one-pot calibration, compensation, and spectral unmixing, and methods for their use.

### CROSS-REFERENCE TO RELATED APPLICATIONS

This application is a continuation of International Application No. PCT/US2023/072659, filed Aug. 22, 2023, which claims priority to and the benefit of U.S. Provisional Patent Application No. 63/400,039, filed on Aug. 22, 2022. Each of the aforementioned applications are incorporated by reference herein in its entirety for all purposes.

### FIELD OF THE DISCLOSURE

The present disclosure relates to compositions of matter and methods that allow for calibration, compensation, and spectral unmixing in a single well.

### BACKGROUND OF THE DISCLOSURE

Flow cytometry, hematology, and image-based cytometry are techniques that allow for the rapid separation, counting, and characterization of individual particles and are routinely used in clinical and laboratory settings for a variety of applications. The technology typically relies on directing a beam of light onto a focused stream of liquid. In one form, a number of detectors are then aimed at the point where the stream passes through the light beam: one in line with the light beam (e.g., Forward Scatter or FSC; also known as Axial Light Loss or ALL) and several perpendicular to it (e.g., Side Scatter or SSC). FSC generally correlates with the particle volume and SSC generally depends on the complexity, or granularity, of the particle (i.e., shape of the nucleus, the amount and type of cytoplasmic granules or the membrane roughness). As a result of these correlations, different specific particle types (i.e., cells, extracellular vesicles) exhibit different FSC and SSC, allowing them to be distinguished from one another.

These measurements comprise the basis of cytometric analysis. In some forms of analysis, cells are also imaged and the descriptive features of the cells, such as size/shape/volume and, in some cases when combined with detection reagents, biochemical features, are recorded. In other forms of analysis, interferometry, particle tracking analysis, and electrical perturbations are used to measure particle characteristics. In additional forms, the cells are labeled with reagents that detect the presence of biomarkers (or nucleic acids) that allow for multiplexed measurement of object features and characteristics.

The use of fluorescent molecules, such as fluorophore-labeled antibodies, in flow cytometry is a common way to study cellular characteristics. For the purposes of clarity, fluorophore and fluorochrome are used interchangeably in the specification. A fluorophore may also be referred to as a tag, dye or stain. Within these types of experiments, a labeled antibody is added to the cell sample. The antibody then binds to a specific molecule on the cell surface or inside the cell. Finally, when the laser light of the appropriate wavelength strikes the fluorophore, a fluorescent signal is emitted and detected by the flow cytometer.

However, when using fluorophores, it can be difficult to delineate between different target species based solely on corresponding fluorescent signals, which may overlap and muddy the ability to assign meaningful values to each target species. The present disclosure addresses this shortcoming.

### SUMMARY OF THE INVENTION

In one aspect, the present disclosure provides for a composition for compensation or spectral unmixing calculations in a single well comprising a first polymer particle having a first biomarker found on a target cell and a second polymer bead comprising a second biomarker found on the target cell. In another aspect, the present disclosure provides for a method of calibrating a cytometric device comprising mixing such composition in a single well with antibody-fluorophores conjugates specific for each biomarker in a cytometric device, measuring the fluorescence signal of the mixture, deconvoluting the fluorescence signal of the first and second antibody-fluorophore bound polymer beads from the measured fluorescence signal of the mixture using known fluorescent signals of the first and second antibody-fluorophore conjugates in order to calculate a compensation or spectral unmixing matrix and calibrating the cytometric device.

In one aspect, the present disclosure provides for a composition for compensation or spectral unmixing calculations in a single well comprising a first polymer particle having a first biomarker found on a target cell and a second polymer bead comprising a second biomarker found on the target cell, wherein a first antibody-fluorophore conjugate specific for the first biomarker and a second antibody-fluorophore conjugate specific for the second biomarker are pre-bound or pre-conjugated to the first biomarker and second biomarker. In another aspect, the present disclosure provides for a method of calibrating a cytometric device comprising mixing such composition in a single well in a cytometric device, measuring the fluorescence signal of the mixture, deconvoluting the measured fluorescence signal of the first and second antibody-fluorophore bound polymer beads from the measured fluorescence signal of the mixture using known fluorescent signals properties of the first and second antibody-fluorophore conjugates in order to calculate a compensation or spectral unmixing matrix and calibrating the cytometric device.

In a preferred embodiment, the polymer particles comprise hydrogels which are substantially similar to the auto-fluorescence and other optical properties of the target cell.

The invention allows for compensation or spectral unmixing calculations to be done in a single reaction. The invention also allows for FMO calculations to be performed using a single reagent mixture.

The present disclosure also provides for computational methods of performing fluorescent compensation and spectral unmixing using a single reaction vessel wherein the plurality of individual bead populations are combined with a complete staining panel mixture in a single tube. The individual bead populations may be modified, a priori, with the fluorophores used in a given panel, or they may be prepared such that they specifically bind to each individual antibody-fluorophore conjugate from the staining panel mixture. The present disclosure provides for methods to derive deconvoluted data from the single reaction vessel based upon the intrinsic or pre-determined fluorescent properties of an individual fluorophore/fluorochrome in order to generate input data required for compensation or spectral unmixing calculations. The present disclosure also provides for methods to derive FMO control calculations from a one-mixture antibody staining panel. The present disclosure also provides for software-driven method for automatically calculating the individual compensation and spectral unmixing calculations from this approach.

In another embodiment, the fluorescent features can be directly-conjugated to the polymer beads. For example, rather than create biochemically-distinct bead populations in the mixture that specifically bind to individual reagents within a staining panel, the beads can be labeled a priori with fluorophores from a staining panel (or combinations of fluorophores or pre-bound to the antibody-fluorophore conjugates) such that they can be easily deconvoluted for compensation or FMO calculations.

### BRIEF DESCRIPTION OF THE FIGURES

FIG. 1A and FIG. 1B illustrate the general concept of fluorescent compensation and spectral unmixing, highlighting the differences between (A) traditional and (B) one-pot methods. The present invention results in dramatic time savings for high complexity assays (e.g. 30 color calculations).

FIG. 2A and FIG. 2B illustrate traditional compensation bead workflows requiring separate tubes, and why a combined staining panel approach does not work using existing reagents. Standard compensation beads bind to a wide range of detection antibodies (depicted as “Reagent”) in a staining panel. Therefore, the reagents must be separated into individual tubes in order to calculate compensation data (A). If a staining panel is combined into a mixture and then added to compensation beads, multiple fluorophores would bind each bead, making deconvolution and compensation or spectral unmixing calculations impossible (B).

FIG. 3 depicts fluorescence spillover and compensation adjustment required to calculate appropriate emission signal data. In traditional workflows, each fluorophore-antibody combination must be separated into individual tubes and physically deconvoluted in order separate the signals for compensation or spectral unmixing calculations. This is largely driven by the fact that the particles used in traditional compensation calculations (whether cells or traditional compensation beads) will indiscriminately bind to most, if not all, of the antibodies in a given panel so they cannot be separated into distinct events.

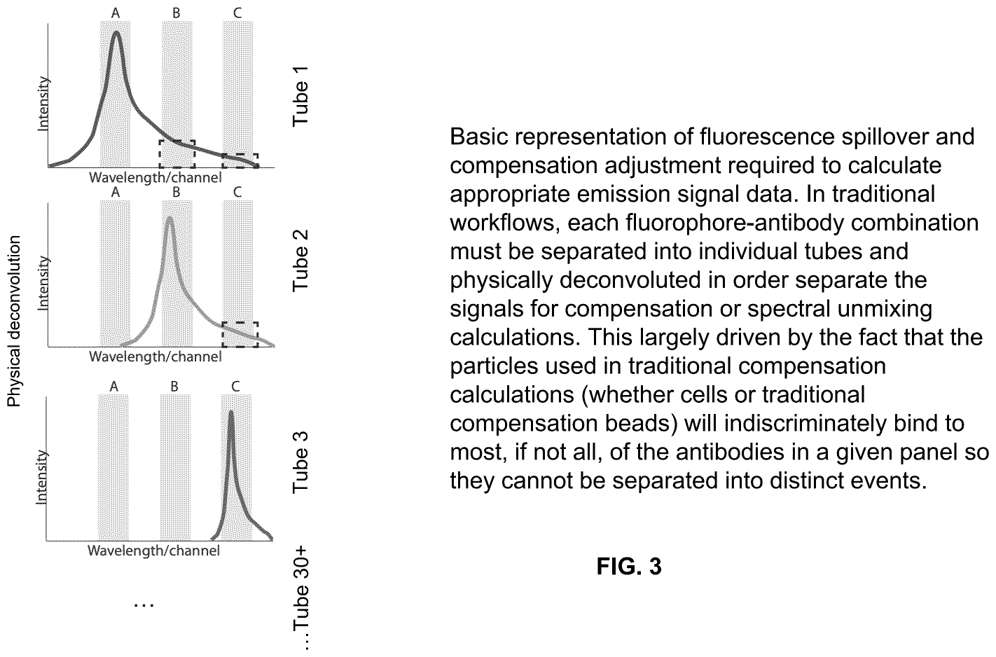

FIG. 4 illustrates an example of a one-pot compensation or unmixing method of the present disclosure where the individual bead populations are pre-modified with fluorochromes or the bead populations are modified to bind to individual reagents (antibody-fluorophore conjugates) from a mixed staining panel. Biomarker-modified compensation beads will only bind to a single antibody in a reagent cocktail, allowing the user to add a complete staining panel mixture to a single tube and deconvolute individual fluorescent signals from the mixture for compensation or spectral unmixing calculations. Individual fluorophore signals can be deconvoluted for compensation or spectral unmixing calculations because each bead will only bind to a specific reagent in the mixture. This results in a dramatically simplified workflow.

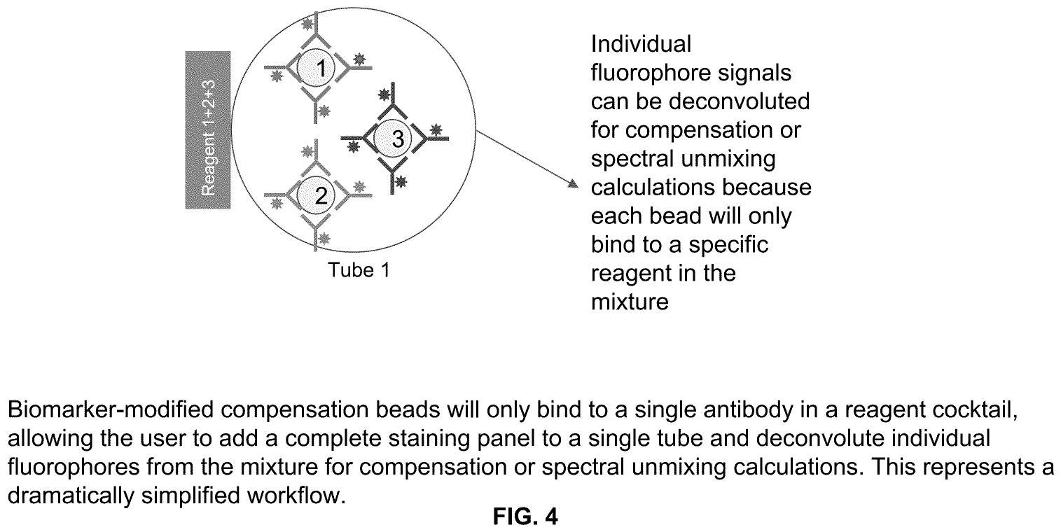

FIG. 5 illustrates how a user can add a pre-aliquoted mixture of selectively binding biomarker beads (for example, a complete staining panel) in a one-pot reaction to computationally deconvolute individual fluorophore profiles to calculate compensation or spectral unmixing. This is enabled by the fact that each individual bead in the mixture will bind only to one antibody from the cocktail. Software can then selectively isolate the profile information by selecting local maxima (FIG. 13), for example, allowing for in silico deconvolution vs. physical tube-derived deconvolution (FIG. 2).

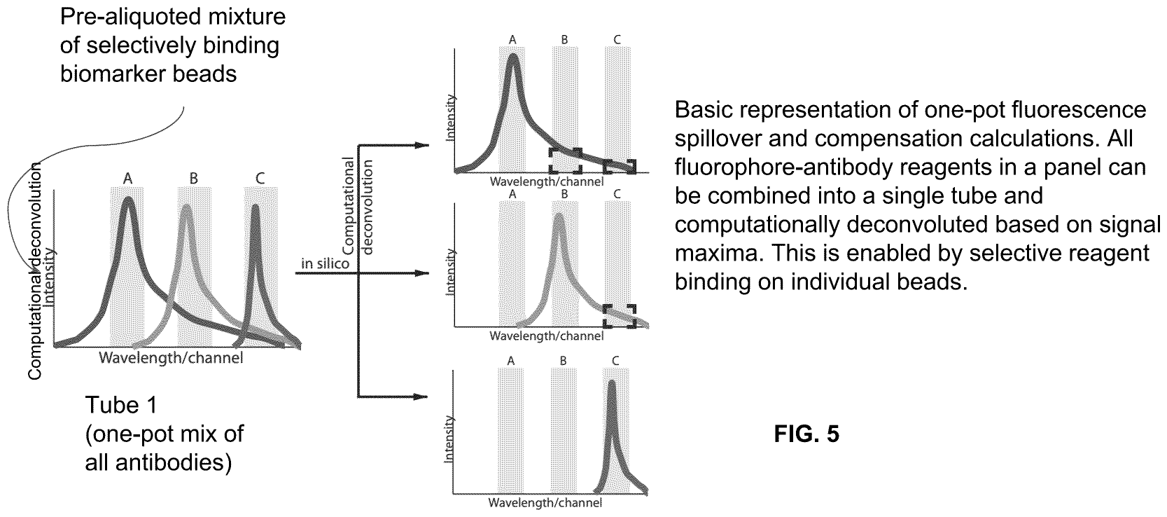

FIG. 6A and FIG. 6B illustrate the differences between (A) a typical fluorescence minus one (FMO) staining panel workflow compared to (B) an FMO panel generated using the reagents and approach described in the present disclosure. In the typical workflow, the combinatorial mixture of antibodies must be generated by the user and then applied to the binding particle, typically patient cells. This results in dramatic time savings for high complexity (e.g. 30 color) assays.

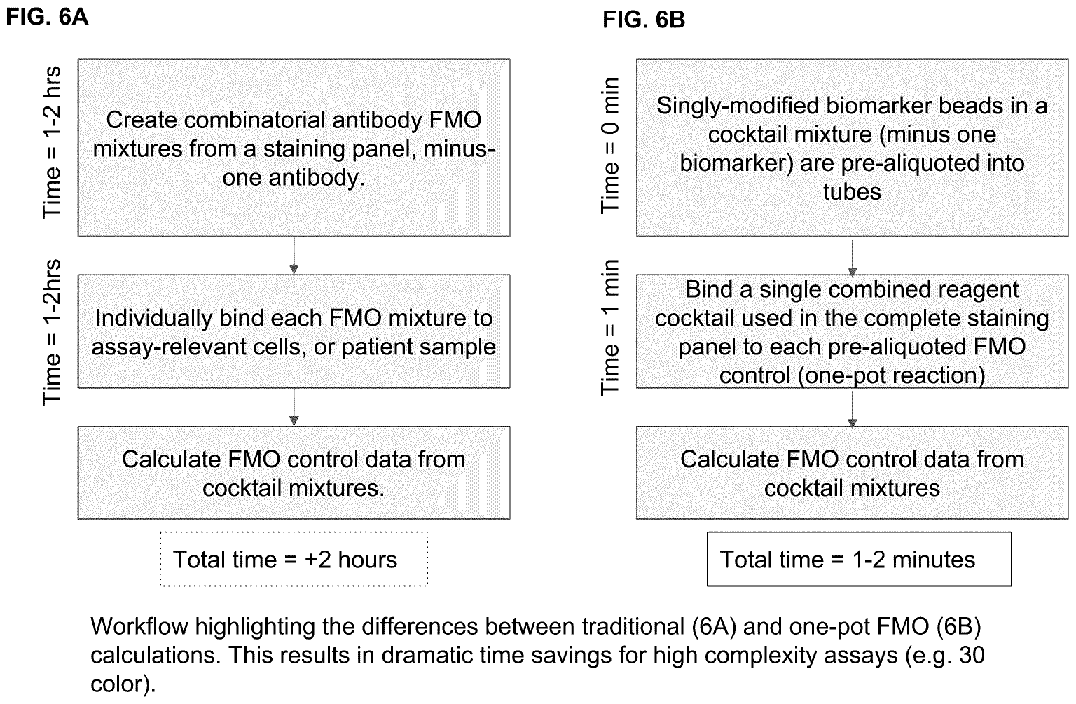

FIG. 7 illustrates the physical process of generating FMO data for an experiment, by creating the combinatorial mixture of reagents (minus one) used in a staining panel. Standard FMO workflows require the user to laboriously create the combinatorial mixture of all antibodies in a staining panel (minus one) in order to calculate the FMO noise floor. This is driven by the fact that the reagents or cells used for FMO calculations will bind to all antibodies in a cocktail.

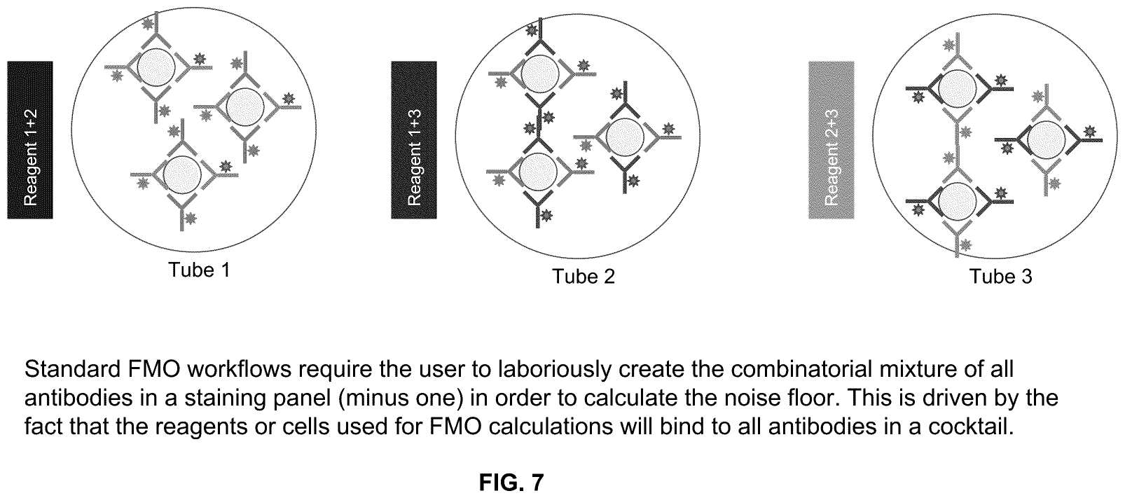

FIG. 8 depicts the importance of FMO calculations, especially for dimly or poorly expressed biomarkers, to ensure accurate data generation. In this simplified panel, there are three staining antibodies in the collection, each emitting in a unique channel. Due to fluorescence spillover, the true noise floor or lower limit of detection is highlighted by the dotted line. Any signal below that dotted line cannot be reliably measured as the signal is generated from orthogonal antibodies in the panel, and not a true biological representation of the presence of the target biomarker for that channel. This is especially important for poorly expressed or “dim” biomarker sets.

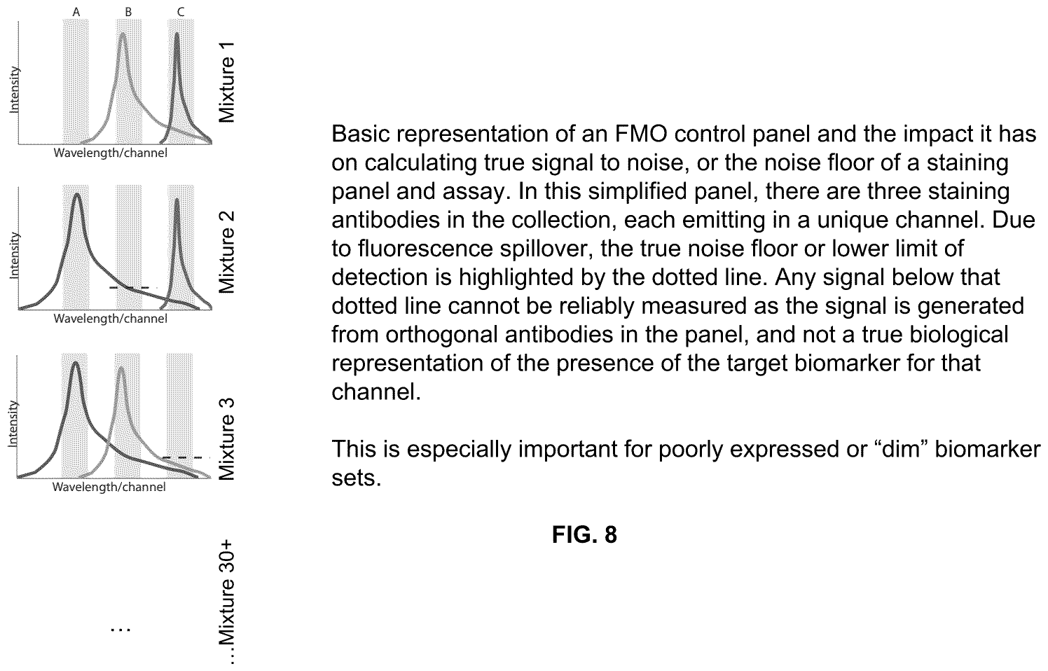

FIG. 9 illustrates a one-reagent-mixture FMO control workflow where the user only prepares one master mix of staining antibodies for a staining panel. The user then applies that single mixture to pre-aliquoted combinatorial mixtures of FMO binding beads, greatly improving operator efficiency and reducing errors. The FMO binding beads are pre-aliquoted to contain the full panel (minus one) enabling the user to create only one master mix of reagents, saving significant time compared to traditional workflows.

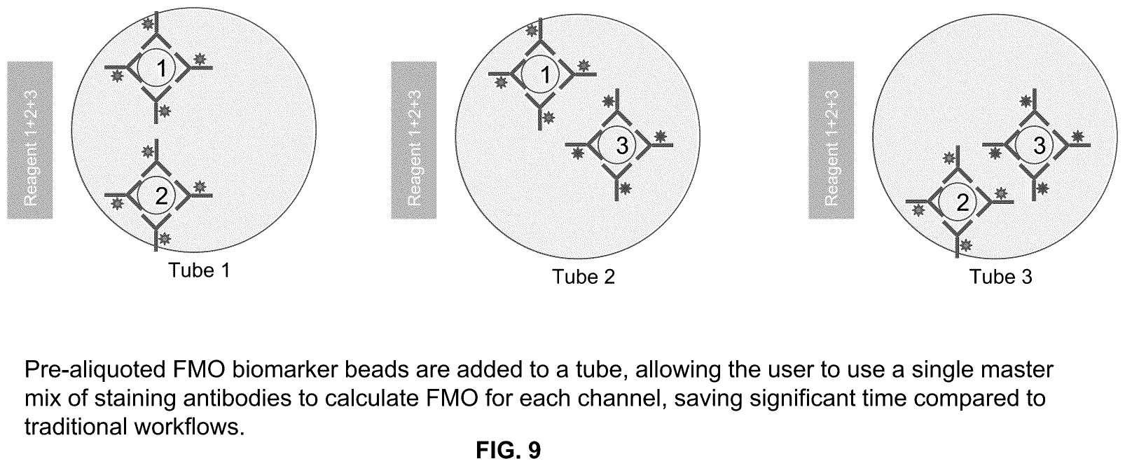

FIG. 10 illustrates a one-mixture FMO control panel and the impact it has on calculating true signal to noise, or the noise floor of a staining panel and assay. In this form, the combinatorial FMO biomarker beads are combined, a priori, allowing the user to make a single cocktail that needs to be added to each tube of mixed biomarker beads. Each of these mixtures is missing a unique biomarker bead, acting like an FMO control. This dramatically speeds up the workflow for calculating FMO controls for complex panels. This also eliminates the need to use actual patient samples or cells for FMO calculation.

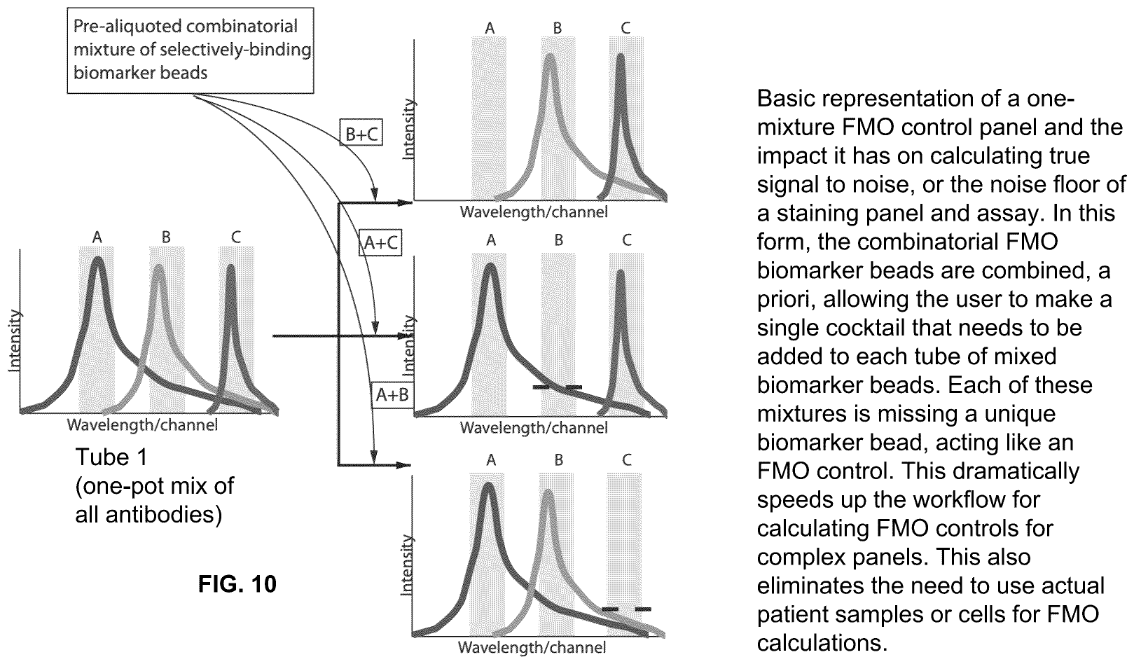

FIG. 11 illustrates a traditional compensation data workflow with 6 antibodies from a TBNK panel. Typically, individual antibody-fluorophore conjugates must be mixed and bound with compensation beads in separate tubes to generate spectra used for compensation or spectral unmixing calculations, as shown. (PerCP-Cy5.5, PE Cy7, APC-Cy7, FITC, PE, APC).

FIG. 12 depicts six unique antibody-fluorophore conjugates mixed in the same tube and analyzed together. Whether traditional compensation beads or the beads of the present invention are used, the signal from the mixture will be the same. However, this signal cannot be deconvoluted using traditional compensation beads because each bead binds to all of the reagents in a given mixture. This precludes effective compensation or spectral unmixing calculations. However, when the compositions and methods of the present invention are used, the independent signals can be deconvoluted from the mixture and compensation or spectral unmixing calculations can be done. In this example a spectral cytometer is used but any traditional cytometer can also be used.

FIG. 13 depicts the process of selecting maxima to deconvolute individual fluorophore signals from a combined mixture of the beads described in the disclosure. This enables the data, from FIG. 12 for example, to be separated into individual channels. For this basic example, the maxima of each fluorescence channel was used to select the individually modified beads.

FIG. 14 depicts the individual fluorophore signals successfully deconvoluted from the one-pot reaction mixture containing individually modified beads, enabling a single-tube compensation matrix/spectral unmixing calculations to be performed. Such individual fluorophore signals are indistinguishable from the data generated by individual separated tubes in FIG. 11.

FIG. 15A and FIG. 15B depict the resulting data from (B) a spectrally-unmixed TBNK staining panel using the one-pot bead mixture described in this disclosure, which is indistinguishable from (A) the individually separated tubes used for spectral-unmixing.

### DETAILED DESCRIPTION OF THE INVENTION

### Definitions

As used herein, the indefinite articles “a” and “an” and the definite article “the” are intended to include both the singular and the plural, unless the context in which they are used clearly indicates otherwise. “At least one” and “one or more” are used interchangeably to mean that the article may include one or more than one of the listed elements.

As used herein, the terms “polymer bead” and “polymer particle” may be used interchangeably.

Unless otherwise indicated, it is to be understood that all numbers expressing quantities, ratios, and numerical properties of ingredients, reaction conditions, and so forth, used in the specification and claims are contemplated to be able to be modified in all instances by the term “about”. For instance, throughout this application, the term “about” may be used to indicate that a value includes the inherent variation of error for the device or the method being employed to determine the value, or the variation that exists among the samples being measured. Unless otherwise stated or otherwise evident from the context, the term “about” means within 10% above or below the reported numerical value (except where such number would exceed 100% of a possible value or go below 0%). When used in conjunction with a range or series of values, the term “about” applies to the endpoints of the range or each of the values enumerated in the series, unless otherwise indicated. As used in this application, the terms “about” and “approximately” are used as equivalents.

There is a general trend in the art to increase the number of biophysical features being measured in a single experiment or assay tube. For the purposes of this application, the terms assay tube, well, container, pot, tube and reaction vessel are used interchangeably. This is commonly referred to as a “staining panel” that is designed to characterize the sample of interest using a set of reagents, typically an antibody that recognizes an epitope, conjugated to a fluorophore. A larger staining panel allows for higher “plex” analysis, greater operator efficiency and reduced sample and analyte requirements to run a complex characterization/phenotyping assay. Exemplary staining panels include the Optimized Multicolor Immunofluorsecence Panels (OMIPs) outlined by the International Society for Advancement of Cytometry.

The complex methodologies involved in fluorescence detection provide significant hurdles for the researcher to consider. This includes the operational time to physically separate each reagent in a staining panel for individual analysis during compensation control set up, which can comprise several hours of labor for complex panels. This also increases the likelihood of operator error in many settings. The further complexities of flow cytometry, combined with the consequent design of experimental protocol and detailed analysis involving numerous fluorophores and fluorescent signals, provide additional obstacles for the efficient utilization of flow cytometry. Proper consideration of spectral overlap that results from use or inclusion of multiple fluorescent materials in different detection systems currently is reactive to these problems as they arise.

As the complexity of a staining panel increases, it becomes more important to distinguish fluorescent or spectral signals from one another, typically by using compensation or spectral unmixing methods, because there is a greater chance that a given set of fluorophores cannot be reliably distinguished from one another. This is known as fluorescent or spectral overlap/spillover/crosstalk, and can confound the interpretation of results. From a first-principles perspective, this effect is driven by the fact that most fluorescent reagents have a range of excitation and emission spectra vs a single wavelength emission profile. Therefore, compensation becomes increasingly important as the complexity of staining panels increases.

### Fluorescence

A fluorophore is a molecule that is capable of fluorescing. In its ground state, the fluorophore molecule is in a relatively low-energy, stable configuration, and it does not fluoresce. When light from an external source hits a fluorophore molecule, the molecule can absorb the light energy. If the energy absorbed is sufficient, the molecule reaches an excited state (high energy); this process is known as excitation. There are multiple excited states or energy levels that the fluorophore can attain, depending on the wavelength and energy of the external light source. Since the fluorophore is unstable at high-energy configurations, it eventually adopts the lowest-energy excited state, which is semi-stable. The excited lifetime (the length of time that the fluorophore is an excited state) is very short; the fluorophore rearranges from the semi-stable excited state back to the ground state, and part of the excess energy may be released and emitted as light. The emitted light is of lower energy, and of longer wavelength, than the absorbed light, thus the color of the light that is emitted is different from the color of the light that has been absorbed. De-excitation returns the fluorophore to its ground state. The fluorophore can absorb light energy again and go through the excited state to ground state process repeatedly.

### Fluorescence Spectra

A fluorescent dye absorbs light over a range of wavelengths and every dye has a characteristic excitation range. This range of excitation wavelengths is referred to as the fluorescence excitation spectrum and reflects the range of possible excited states that the dye can achieve. Certain wavelengths within this range are more effective for excitation than other wavelengths. A fluorophore is excited most efficiently by light of a particular wavelength. This wavelength is the excitation maximum for the fluorophore. Less efficient excitation can occur at wavelengths near the excitation maximum; however, the intensity of the emitted fluorescence is reduced. Although illumination at the excitation maximum of the fluorophore produces the greatest fluorescence output, illumination at lower or higher wavelengths affects only the intensity of the emitted light; the range and overall shape of the emission profile are unchanged.

A molecule may emit at a different wavelength with each excitation event because of changes that can occur during the excited lifetime, but each emission will be within the fluorescence emission spectrum. Although the fluorophore molecules all emit the same intensity of light, the wavelengths, and therefore the colors of the emitted light, are not homogeneous. The emission maximum is the wavelength where the population of molecules fluoresces most intensely. The excited fluorophore can also emit light at wavelengths near the emission maximum. However, this light will be less intense.

Different types of light sources are used to excite fluorophores. Common sources include broadband sources, such as, for example, mercury-arc and tungsten-halogen lamps. These lamps produce white light that has peaks of varying intensity across the spectrum. When using broadband white light sources it is necessary to filter the desired wavelengths needed for excitation; this is most often done using optical filters. Optical filters selectively allow light of certain wavelengths to pass while blocking out undesirable wavelengths. A bandpass excitation filter transmits a narrow range of wavelengths and may be used for selective excitation. Laser excitation sources may also be used. Lasers provide wavelength peaks that are well-defined, selective, and of high intensity allowing more selective illumination of the sample. High-output light-emitting diodes (LEDs) provide selective wavelengths, low cost and energy consumption, and long lifetime. Single-color LEDs are ideal for low-cost instrumentation where they can be combined with simple longpass filters that block the LED excitation and allows the transmission of the dye signal. However, the range of wavelengths emitted from each LED is still relatively broad and also may require the use of a filter to narrow the bandwidth. Further information regarding fluorescence spectra may be found in The MolecularProbes® Handbook—A Guide to Fluorescent Probes and Labeling Technologies, incorporated herein by reference in its entirety.

### Fluorescence Detection

In a traditional or conventional flow cytometer and other instruments that employ a multiplicity of photodetectors to detect a multiplicity of dyes, the collected light is separated into specific ranges of wavelengths, typically by a system of frequency-dependent filters and dichroic mirrors, such that the light detected by a particular photodetector or photomultiplier tube (PMT) is limited to a predefined range of wavelengths, sometimes referred to as a detection channel. The detection channels and dyes are selected such that the peak of the emission spectrum of each dye is within the frequency range of a different detection channel, e.g., each detection channel detects primarily the emission from a single dye. However, because of the breadth of the emission spectra of fluorescent dyes, typically a dye will fluoresce in more than one detection channel and, thus, measurements of dye fluorescence are not independent. The emission of one dye in detection channels intended for the detection of other dyes is referred to by a number of terms, such as spillover, spectral overlap, and crosstalk.

Spectral flow cytometry is a technique based on conventional flow cytometry where a spectrograph and multichannel detector (e.g., charge-coupled device (CCD)) is substituted for the traditional mirrors, optical filters and PMTs in conventional systems. In the spectral flow cytometer, fluorescent light is collected and displayed as a spectrograph, either directly or through an optical fiber, where the whole light signal is dispersed and displayed as a high-resolution spectrum on the CCD or coupled into one or more multichannel detectors for detection.

For proper data interpretation, the fluorescent light recorded from one fluorescent source must be distinguished from that recorded from other fluorescent sources. For that reason, the ideal fluorophore has a fluorescence emission profile of a very intense, narrow peak that is well separated from all other emission peaks. However, typical fluorophores have broad emission peaks that may overlap or spillover. This overlap or spillover may compromise data and analysis.

Background fluorescence, which may originate from endogenous sample constituents (autofluorescence) or from unbound or nonspecifically bound reagents, may compromise fluorescence detection. The detection of autofluorescence can be minimized either by selecting filters that reduce the transmission or detection of autofluorescence, but by doing so, the overall fluorescence intensity detection is compromised. A full spectrum flow cytometer will detect autofluorescence.

### Calibration

Most assays require some form of calibration and set up to ensure accurate performance when analyzing a biological sample. An example of calibration includes setting gains and voltages for detection, measuring inter-laser drop delay, and ensuring linearity in detecting fluorescence signals. In many instances, the ideal calibration and set up reagent looks and acts like the biological particle being analyzed. This helps to ensure similar performance in a diagnostic instrument, while not introducing artifacts into measurement processes.

Most synthetic or polymer products used in cellular analysis are made of polystyrene, an opaque polymer with high refractive index that generally has a fixed forward and side scatter profile based on the diameter of the particle. This high refractive index is distinct from biological particles such as cells and extracellular vesicles, which are semi-transparent and allow internal features, such as organelles in the case of cells, to be optically resolved and measured. In particular, extracellular vesicles often have a low refractive index relative to their diameter. Polystyrene is also hydrophobic and has a high elastic modulus, two features that also distinguish it from cellular and biological material. In some forms, silica (SiO2) is used as a surrogate for a biological particle. While the refractive index of silica is closer to that of a typical extracellular vesicle, there are still substantial differences in optical response, requiring interpolation when used as a calibration reagent.

Together, these characteristics make polystyrene and silica particles less than ideal when aiming to create a control and calibration reagent for biological particle measurement. As a result, many practitioners use cellular material or biologically-derived particles (e.g. lipid nanoparticles) as a process and reference control, prior to measuring a sample. Cellular and biological material suffers from other drawbacks, including poor stability, limited and complex supply chain, high batch-to-batch variability, and high cost of production. In addition, the material typically has stringent cold-chain handling requirements, limiting the scope and application of the control.

### Compensation and Spectral Unmixing

Another example of calibration includes fluorescence compensation, where the excitation and emission spectra of a given fluorophore is distinguished from potentially overlapping sets of fluorophores used in the same set of experiments. An additional example of calibration includes spectral unmixing, where the spectral response (sometimes referred to as a fluorophore's emissions or fingerprint or signature or pattern) of a given fluorophore is distinguished from potentially overlapping sets of fluorophores used in the same set of experiments. For conventional cytometers, the process is referred to as “compensation”, while for spectral cytometers it is named “spectral unmixing”. While compensation and spectral unmixing share the same conceptual goal, they are based on different mathematic calculations.

Ideally, when one uses a dye in an experiment, its emission spectrum will be narrow enough that fluorescence from that dye is only detected by a single detector in the instrument. In practice, because of the broad emission spectra of available fluorochromes, the dye being used will likely emit significant amounts of fluorescence in several different detectors. In other words, the light reaching a given detector consists of the signals from multiple fluorochromes. Compensation is the process of transforming the data such that the values from a single detector come from an individual dye. In order to separate these signals, or compensate for the overlapping emissions, a percentage of each overlapping emission is subtracted from the target emission. Traditionally, this compensation was performed by the instrument during acquisition. However, modern instruments are capable of storing the data in uncompensated form and compensation can be applied by the analysis software. In an exemplary calculation, Compensated Parameter 2 Fluorescence equals Observed Parameter 2 Fluorescence minus 5% of Observed Parameter 1 Fluorescence.

Compensation involves creating two matrices. The spillover matrix represents the percentage of the signal from a given channel that spills into adjacent channels. The compensation matrix, used to correct for the spillover, is the inverse of the spillover matrix.

In embodiments, the target parameter is the parameter that is detecting signal (potentially from multiple sources). The source parameter is the primary parameter that you want the signal to be in, but that dye is also bleeding (potentially) into multiple targets. In other words, we are subtracting the percentage of the source that is “bleeding” into the target. In the example given above, Parameter 2 is the target and Parameter 1 is the source. A family of sources and targets is called a compensation definition. A compensation definition describes all the ways that fluorescence from different channels affect each other under a given set of conditions and is equivalent to a single compensation matrix. Typically, the instrument user will set the gains for all the channels at the beginning of the experiment and use these settings for the duration of the experiment. Thus, the compensation definition would apply for the entire experiment.

With spectral cytometry instruments, a continuous, high resolution, optical spectrum is collected for each event in the sample. The spectrum is the sum of the spectra derived from all the dyes present on the event of interest. Spectral Unmixing is the process of transforming the data to determine the contribution of each dye to the total signal. Several mathematical models can be used to perform the spectral unmixing calculation, including the Ordinary Least Square method. The Ordinary Least Square method assumes a linear contribution of unchanging reference spectra to the mixture spectra of an unknown sample. In turn, the calculation allows for the estimation of the contribution of each spectrum (i.e. each dye).

Ordinary Least Square uses a linear decomposition algorithm to solve the equation:

$$Y = A C + E$$

For the sake of simplicity, the term “E”, which is the random measurement error, can be initially ignored and the remaining terms can be brought into focus, where Y is the measured spectrum matrix (i.e. a 1-column matrix containing the spectrum of the event of interest), A is the reference spectra matrix (i.e. a n-columns matrix containing the spectrum of each reference dye), and C is the concentration matrix (i.e. a 1-row matrix containing the contribution values of each dye to the total measured spectrum). Given a series of reference spectra (A), and a measured spectrum (Y), this method allows for the estimation of the term C, and thus the contribution of each dye to the total signal intensity of the event of interest.

The term “compensation” as used herein refers to correction of the emission signal to accurately estimate the fluorescence signal for a given fluorophore. The term “spectral unmixing” as used herein refers to separating the emission spectra to accurately estimate the spectral signal for a given fluorophore. Both compensation and spectral unmixing are directed to removal of spillover signals from other fluorophores. Spectral unmixing handles data from more detectors than compensation.

Methods of compensation and spectral unmixing, as introduced above, are known in the art. Such methods involve adjustment of the signal measured by each photodetector by an amount calculated to compensate for the contribution from dyes other than the primary dye to be detected. Examples in the field of flow cytometry include Bagwell et al., 1993, “Fluorescence Spectral Overlap Compensation for any Number of Flow Cytometer Parameters”, Ann. N.Y. Acad. Sci. 677: 167-184; Roederer et al., 1997, “Eight Color, 10-Parameter Flow Cytometry to Elucidate Complex Leukocyte Heterogeneity”, Cytometry 29: 328-339; and Bigos et al., 1999, Cytometry 36: 36-45; Verwer, 2002, BD FACSDiVa™ Option for the BD FACSVantage SE Flow Cytometer White Paper, and U.S. Pat. No. 6,897,954, each incorporated herein by reference. WinList™ (Verity Software House, Topsham, Me.), Orfeo ToolBox (CNES), FCS Express (De Novo Software, Pasadena, California) and FlowJo 5.7.2 software (Tree Star, Inc., Ashland, Oreg.) are a stand-alone software packages that allow software compensation on stored data files produced by a flow cytometer.

Typically, the amount of fluorescence compensation required is determined experimentally using compensation control beads (bound to a single antibody-fluorophore conjugate) or single-color particles dyed with one of the fluorescent dyes used in the assay. The fluorescence signal of each bead is measured in each of the channels, which directly provides a measure of the signal overlap in each of the channels. One method of measuring signal overlap of fluorescently labeled antibody reagents (e.g., detection reagents) in each of the detection channels is using BD™ CompBeads compensation particles (BD Biosciences, San Jose, Calif.). The particles, which are coated with anti-Ig antibodies, are combined with a fluorescently labeled antibody reagent, which becomes captured on the surface of the bead, to produce a particle labeled with the fluorescent dye. The signal overlap of the dye is determined by measuring the emission of the labeled particle in each of the detection channels. The measurement typically is made relative to the emission spectrum from the unlabeled particle. This process becomes more challenging as the number of fluorophores used in an assay increases, thereby causing more signal overlap. There is a need for methods and compositions that can improve spectral unmixing and compensation (and thereby improve resolution) in multi-parameter flow cytometry.

In simplified form, a percentage of fluorescence is subtracted from one channel measuring a fluorophore from a second channel measuring the fluorescence of the second (or multiple) fluorophore such that the contribution of the incidental fluorescence is removed. Every fluorophore combination that shows spectral overlap must be compensated. To determine the amount of compensation required to correct the fluorescence data, single-color samples (either aliquots of the cell sample or microspheres that bind to all of the antibodies in a staining panel, are stained with each fluorophore separately, in individual tubes) are utilized and analyzed with the experimental samples, which are typically then stained with multiple fluorophores.

Compensation is typically performed by using polystyrene beads that are modified such that they bind to detection antibodies, often via the Fc region. In this form, compensation beads will indiscriminately bind to most if not all of the antibodies used in an experiment, allowing the operator to measure the fluorescence/spectral profile of a given antibody-fluorophore/fluorochrome conjugate when they are measured in isolation, in individual tubes.

In current form, the operator must aliquot individual tubes of beads and add individual antibodies from a staining panel to each tube, separately, in order to deconvolute individual signals for fluorescent compensation or spectral unmixing. As the complexity of the staining panel increases, this process can sometimes take hours to complete and is highly prone to operator/user error due to the number of pipetting steps that are required.

Similarly, fluorescence minus one (FMO) controls are equally important to determine the true signal to noise of a given biomarker/channel, especially when there are differing levels of expression for a given biomarker in a multiplexed assay. In these instances, the combinatorial cocktail of all antibodies used in a given panel, minus one, must be mixed and bound to a target cell in order to determine the noise floor or lower limit of detection for a given assay and staining panel. Briefly, due to spillover effects, the measured amount of fluorescence in a particular channel may not be representative of a biological measurement of a biomarker in that channel. Instead, it is often representative of inadvertent fluorescence spillover from adjacent channels that measure other biomarkers. In order to determine true biological signal from noise, FMO controls are critical. This is especially important for poorly expressed or “dim” biomarkers which have very low signal intensity. This process can be extremely time consuming, involving the combinatorial mixing of antibodies for a panel into individual tubes and relies, more often than not, on the actual cells being assayed. In circumstances where there is limited cellular material (e.g. primary cells from a patient or rare disease) FMO controls become exceedingly difficult to perform accurately.

Therefore, there is a need in the art for synthetic compositions that allow for more efficient and less error-prone compensation and FMO process controls in order to properly set up an analytical device for multiplexed cytometric analysis.

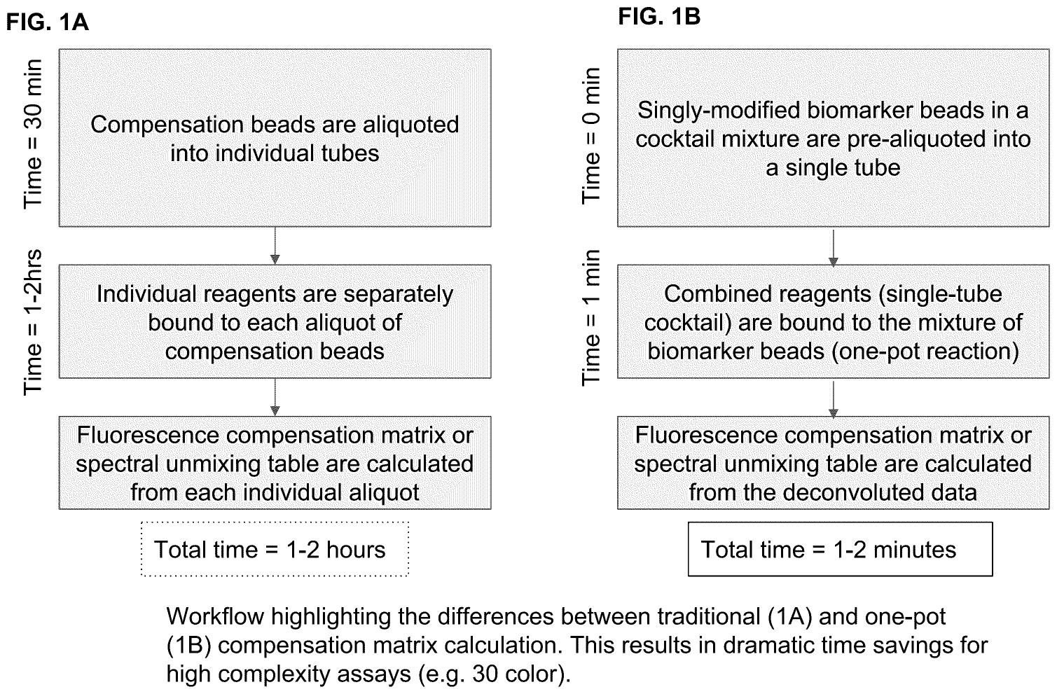

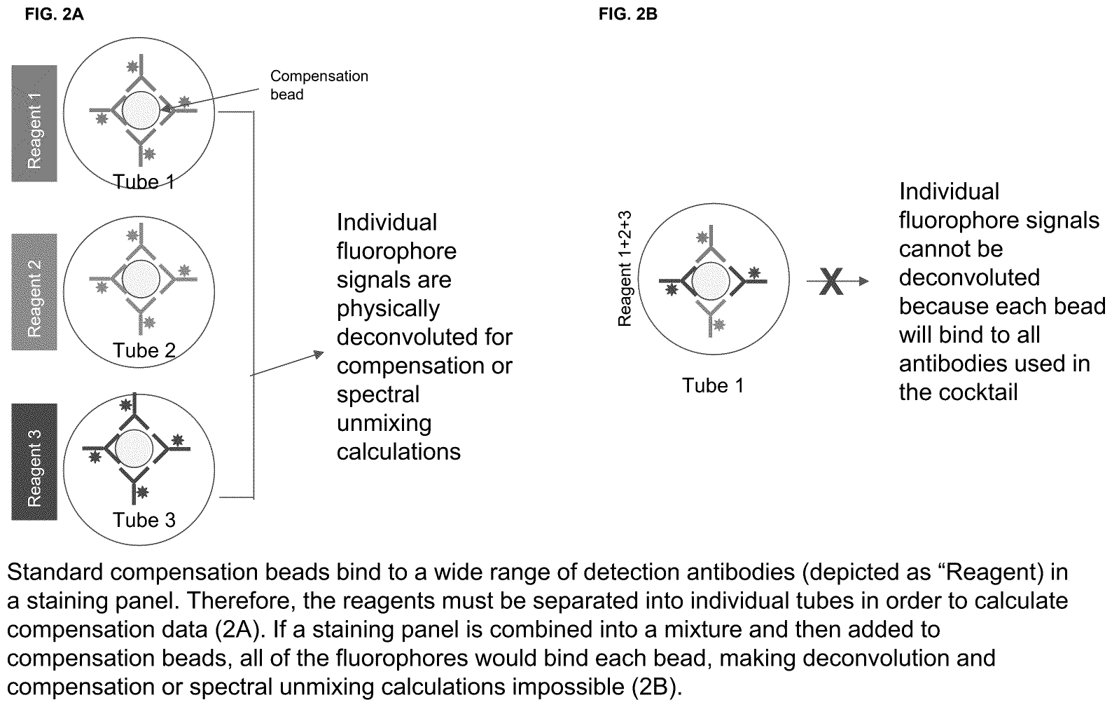

Referring now to the Drawings, as shown in FIG. 1 and FIG. 2, compensation is typically performed by separating individual fluorophores/reagents into distinct tubes containing compensation beads that are designed, for example, to bind to a common region of the antibody reagents used in a staining panel, such as the Fc region. The individual fluorophores must be physically separated in tubes in order to deconvolute the fluorescent signals from antibody-fluorophore conjugates used in a staining panel because each individual compensation bead will bind to multiple distinct antibodies used in a staining panel. As a result, an individual compensation bead may have different fluorophores attached if it were added to a mixture of reagents with distinct fluorophores. For example, an individual compensation bead may have an anti-CD4-FITC and an anti-CD8-Texas Red both attached to such individual compensation bead. This would preclude compensation or spectral unmixing from being calculated effectively. Therefore, each bead and detection antibody combination must be physically separated in order to deconvolute the signal associated with a given fluorophore within a staining panel. This process is extremely labor intensive when working with complex panels and can take hours of operator time. In addition, it is a key source of operator error in large-scale diagnostic settings such as reference labs. This lengthy and labor-intensive process, while critical for ensuring accurate assay performance, increases the cost of clinical trials and research pipelines significantly. As an alternative to modified polystyrene beads, users may often use cellular material as a surrogate for compensation and unmixing set up. Biologically-derived calibration products are unstable, and suffer from batch to batch variability, introducing noise into measurements and causing disparities in the interpretation of diagnostic data. In addition, both standard compensation beads and the cells used in an assay typically bind to most, if not all of the reagents used in a staining panel, requiring individual tubes to be separated for compensation and unmixing calculations as described above.

Accordingly, the present disclosure provides for compositions comprising a hydrogel or polymer particle, wherein the particle has been modified to bind to an individual antibody in a staining panel, but not others. Alternatively, the particle may also be pre-modified with the same fluorophore (or antibody-fluorophore conjugate) used in a staining panel to achieve the same effect. Next, the individual beads comprising the biomarkers representing the full repertoire of a staining panel are combined into a single tube, a priori. In one form, pre-modified fluorophore (or antibody-fluorophore conjugate) beads are directly used in an automated deconvolution and compensation or unmixing process where the signal of an individual bead can be isolated using the expected fluorescent or spectral maxima, generating data that can be used for compensation or unmixing calculations. In another form, the user can add their staining panel, in its entirety, to the multitude of beads that are designed to individually bind to the biomarkers recognized by antibodies in the staining panel, in a one-pot, single-tube reaction in order to generate the same data, using the same deconvolution process.

The present disclosure also provides for methods of combining individually-modified beads in a way such that the user can perform FMO controls using a single mixture of antibodies. Traditionally, for FMO calculations, it is useful to produce the combinatorial mixture of all antibodies used in a staining panel, minus one, in order to determine the noise floor of a given assay and determine true signal to noise. In practice, antibodies in a staining panel will produce some overlapping signal in different unintended channels. When cells are stained with a mixture lacking a specific antibody-fluorophore for a given channel, any signal in that channel represents a false-positive noise floor or true biological lower limit of detection, which can be determined using FMO controls. This process is extremely labor intensive when performing a high complexity staining assay as the full combinatorial matrix of FMO staining antibody cocktails must be prepared by the user, which can take hours of time to complete for complex panels, leading to user error and extensive operator cost and fatigue. The present disclosure provides for methods of performing FMO calculations where the user can add a single complete mixture of a staining panel to tubes that already contain individually-modified beads, minus one bead\epitope type for a given panel. By providing the tubes containing the plurality of individually-modified beads, minus one, the user can greatly simplify the process of setting up FMO controls by enabling them to use one antibody cocktail for the entire process, reducing operator error, saving time, and saving cost.

Also provided for is a method of deconvoluting the single well compensation, unmixing, or FMO control reaction, the method comprising a) analyzing the single well data to find fluorescent maxima that correspond to individual fluorophores in a staining panel; b) deconvoluting the individually-modified beads using the local maxima as a cutoff; and c) performing compensation, unmixing, or FMO calculations using the deconvoluted data from individual bead populations, thereby calibrating the cytometric device for analysis of target biological object.

### Hydrogels and Polymers

Particles of the disclosure may comprise a hydrogel or hydrophobic polymer. A hydrogel is a material comprising a macromolecular three-dimensional network that allows it to swell when in the presence of water, to shrink in the absence of (or by reduction of the amount of) water but not dissolve in water.

In another aspect, a polymer particle can comprise non-polystyrene-based material such as PLGA. In other aspects, the polymer particle is generated using polystyrene and latex.

The hydrogels provided herein, in the form of beads/particles, are synthesized by polymerizing one or more of the monomers provided herein. The synthesis is carried out to form individual hydrogel particles. The monomeric material (monomer) in one embodiment is polymerized to form a homopolymer. However, in another embodiment copolymers of different monomeric units (i.e., co-monomers) are synthesized and used in the methods provided herein. The monomer or co-monomers used in the methods and compositions described herein, in one embodiment, is a bifunctional monomer or includes a bifunctional monomer (where co-monomers are employed). In one embodiment, the hydrogel is synthesized in the presence of a crosslinker. In a further embodiment, embodiment, the hydrogel is synthesized in the presence of a polymerization initiator.

The amount of monomer can be varied by the user of the invention, for example to obtain a particular optical property that is substantially similar to that of a target cell. In one embodiment, the monomeric component(s) (i.e., monomer, co-monomer, bifunctional monomer, or a combination thereof, for example, bis/acrylamide in various crosslinking ratios, allyl amine or other co-monomers which provide chemical functionality for secondary labeling/conjugation or alginate is present at about 10 percent by weight to about 95 percent weight of the hydrogel. In a further embodiment, the monomeric component(s) is present at about 15 percent by weight to about 90 percent weight of the hydrogel, or about 20 percent by weight to about 90 percent weight of the hydrogel.

Examples of various monomers and cross-linking chemistries available for use with the present invention are provided in the Thermo Scientific Crosslinking Technical Handbook entitled “Easy molecular bonding crosslinking technology,” (available at tools.lifetechnologies.com/content/sfs/brochures/1602163-Crosslinking-Reagents-Handbook.pdf, the disclosure of which is incorporated by reference in its entirety for all purposes. For example, hydrazine (e.g., with an NHS ester compound) or EDC coupling reactions (e.g., with a maleimide compound) can be used to construct the hydrogels of the invention.

In one embodiment, a monomer for use with the hydrogels provided herein is lactic acid, glycolic acid, acrylic acid, 1-hydroxyethyl methacrylate, ethyl methacrylate, 2-hydroxyethyl methacrylate (HEMA), propylene glycol methacrylate, acrylamide, N-vinylpyrrolidone (NVP), methyl methacrylate, glycidyl methacrylate, glycerol methacrylate (GMA), glycol methacrylate, ethylene glycol, fumaric acid, a derivatized version thereof, or a combination thereof.

In one embodiment, one or more of the following monomers is used herein to form a hydrogel of the present invention: 2-hydroxyethyl methacrylate, hydroxyethoxyethyl methacrylate, hydroxydiethoxyethyl methacrylate, methoxyethyl methacrylate, methoxyethoxyethyl methacrylate, methoxydiethoxyethyl methacrylate, poly(ethylene glycol) methacrylate, methoxy-poly(ethylene glycol) methacrylate, methacrylic acid, sodium methacrylate, glycerol methacrylate, hydroxypropyl methacrylate, hydroxybutyl methacrylate or a combination thereof.

In another embodiment, one or more of the following monomers is used herein to form a tunable hydrogel: phenyl acrylate, phenyl methacrylate, benzyl acrylate, benzyl methacrylate, 2-phenylethyl acrylate, 2-phenylethyl methacrylate, 2-phenoxyethyl acrylate, 2-phenoxyethyl methacrylate, phenylthioethyl acrylate, phenylthioethyl methacrylate, 2,4,6-tribromophenyl acrylate, 2,4,6-tribromophenyl methacrylate, pentabromophenyl acrylate, pentabromophenyl methacrylate, pentachlorophenyl acrylate, pentachlorophenyl methacrylate, 2,3-dibromopropyl acrylate, 2,3-dibromopropyl methacrylate, 2-naphthyl acrylate, 2-naphthyl methacrylate, 4-methoxybenzyl acrylate, 4-methoxybenzyl methacrylate, 2-benzyloxyethyl acrylate, 2-benzyloxyethyl methacrylate, 4-chlorophenoxyethyl acrylate, 4-chlorophenoxyethyl methacrylate, 2-phenoxyethoxyethyl acrylate, 2-phenoxyethoxyethyl methacrylate, N-phenyl acrylamide, N-phenyl methacrylamide, N-benzyl acrylamide, N-benzyl methacrylamide, N,N-dibenzyl acrylamide, N,N-dibenzyl methacrylamide, N-diphenylmethyl acrylamide N-(4-methylphenyl)methyl acrylamide, N-1-naphthyl acrylamide, N-4-nitrophenyl acrylamide, N-(2-phenylethyl)acrylamide, N-triphenylmethyl acrylamide, N-(4-hydroxyphenyl)acrylamide, N,N-methylphenyl acrylamide, N,N-phenyl phenylethyl acrylamide, N-diphenylmethyl methacrylamide, N-(4-methyl phenyl)methyl methacrylamide, N-1-naphthyl methacrylamide, N-4-nitrophenyl methacrylamide, N-(2-phenylethyl)methacrylamide, N-triphenylmethyl methacrylamide, N-(4-hydroxyphenyl)methacrylamide, N,N-methylphenyl methacrylamide, N,N′-phenyl phenylethyl methacrylamide, N-vinylcarbazole, 4-vinylpyridine, 2-vinylpyridine, as described in U.S. Pat. No. 6,657,030, which is incorporated by reference in its entirety herein for all purposes.

The passive optical properties of the polymer beads may be modulated to mimic the passive optical properties of a target cell. Exemplary target cells are included in the non-exhaustive listing in Table 1.

In embodiments, each polymer bead comprises less than 10%, 20%, 30%, or 40% polystyrene by hydrated or dehydrated volume. Depending on their composition, the polymer beads hydrated and dehydrated volume may be identical.

In embodiments, the hydrogel or polymer particle is functionalized to mimic one or more optical properties of a target cell or labeled target cell. In embodiments, the hydrogel or polymer particle comprises one or more high-refractive index molecules. In embodiments, the hydrogel or polymer particle comprises a plurality of high-refractive index molecules. In embodiments, the high-refractive index molecule enables for mimicking of the SSC of a target cell. In embodiments, the high-refractive index molecule is selected from one or more of colloidal silica, alkyl acrylate, alkyl methacrylate or a combination thereof. In embodiments, the high-refractive index molecule is alkyl acrylate, alkyl methacrylate, or both. In embodiments, alkyl acrylates or alkyl methacrylates contain 1 to 18, 1 to 8, or 2 to 8, carbon atoms in the alkyl group. In embodiments, the alkyl group is methyl, ethyl, n-propyl, isopropyl, n-butyl, isobutyl or tertbutyl, 2-ethylhexyl, heptyl or octyl. In embodiments, the alkyl group is branched. In embodiments, the alkyl group is linear.

The three primary modes of deconvolution for flow cytometry are the two passive optical properties of a polymer particle (FSC, corresponding to the refractive index, or RI, and SSC) and biomarkers present on the surface of a given cell type. Therefore, compositions that allow polymer particles, or polymer beads, of the disclosure to mimic specific cell types with respect to these three modes are useful for providing synthetic, robust calibrants for flow cytometry.

In one embodiment, the RI of a disclosed polymer bead is greater than about 1.10, greater than about 1.15, greater than about 1.20, greater than about 1.25, greater than about 1.30, greater than about 1.35, greater than about 1.40, greater than about 1.45, greater than about 1.50, greater than about 1.55, greater than about 1.60, greater than about 1.65, greater than about 1.70, greater than about 1.75, greater than about 1.80, greater than about 1.85, greater than about 1.90, greater than about 1.95, greater than about 2.00, greater than about 2.10, greater than about 2.20, greater than about 2.30, greater than about 2.40, greater than about 2.50, greater than about 2.60, greater than about 2.70, greater than about 2.80, or greater than about 2.90.

In another embodiment, the RI of a disclosed polymer bead is about 1.10 to about 3.0, or about 1.15 to about 3.0, or about 1.20 to about 3.0, or about 1.25 to about 3.0, or about 1.30 to about 3.0, or about 1.35 to about 3.0, or about 1.4 to about 3.0, or about 1.45 to about 3.0, or about 1.50 to about 3.0, or about 1.6 to about 3.0, or about 1.7 to about 3.0, or about 1.8 to about 3.0, or about 1.9 to about 3.0, or about 2.0 to about 3.0.

In some embodiments, the RI of a disclosed polymer bead is less than about 1.10, less than about 1.15, less than about 1.20, less than about 1.25, less than about 1.30, less than about 1.35, less than about 1.40, less than about 1.45, less than about 1.50, less than about 1.55, less than about 1.60, less than about 1.65, less than about 1.70, less than about 1.75, less than about 1.80, less than about 1.85, less than about 1.90, less than about 1.95, less than about 2.00, less than about 2.10, less than about 2.20, less than about 2.30, less than about 2.40, less than about 2.50, less than about 2.60, less than about 2.70, less than about 2.80, or less than about 2.90.

The SSC of a disclosed polymer bead is most meaningfully measured in comparison to that of target cell. In some embodiments, a disclosed polymer bead has an SSC within 30%, within 25%, within 20%, within 15%, within 10%, within 5%, or within 1% that of a target cell, as measured by a cytometric device.

The SSC of a polymer bead in one embodiment, is modulated by incorporating a high-refractive index molecule (or plurality thereof) in the polymer bead. In one embodiment, a high-refractive index molecule is provided in a polymer bead, and in a further embodiment, the high-refractive index molecule is colloidal silica, alkyl acrylate, alkyl methacrylate or a combination thereof. Thus, in some embodiments, a polymer bead of the disclosure comprises alkyl acrylate and/or alkyl methacrylate. Concentration of monomer in one embodiment is adjusted to further adjust the refractive index of the polymer bead.

Alkyl acrylates or alkyl methacrylates can contain 1 to 18, 1 to 8, or 2 to 8, carbon atoms in the alkyl group, such as methyl, ethyl, n-propyl, isopropyl, n-butyl, isobutyl or tertbutyl, 2-ethylhexyl, heptyl or octyl groups. The alkyl group may be branched or linear.

High-refractive index molecules can also include vinylarenes such as styrene and methylstyrene, optionally substituted on the aromatic ring with an alkyl group, such as methyl, ethyl or tert-butyl, or with a halogen, such as chlorostyrene.

In some embodiments, FSC is modulated by adjusting the percentage of monomer present in the composition thereby altering the water content present during polymer bead formation. In one embodiment, where a monomer and co-monomer are employed, the ratio of monomer and co-monomer is adjusted to change the polymer bead's forward scatter properties.

For example, the ratio of monomer and co-monomer can be used to adjust the polymer bead's elasticity (i.e., Young's Modulus) to be substantially similar to the elasticity of the target cell. The ratio of the monomer and co-monomer can change the Young's Modulus for the polymer bead can range from 0.2 kiloPascals (kPa) to 400 kPa, based on the elasticity of the target cell. The elasticity of the polymer bead (e.g., softness or firmness) can affect the function of the target cell with which the polymer bead interacts.

The FSC of a disclosed polymer bead is most meaningfully measured in comparison to that of target cell. In some embodiments, a disclosed polymer bead has an FSC within 30%, within 25%, within 20%, within 15%, within 10%, within 5%, or within 1% that of a target cell, as measured by a cytometric device.

FSC is related to particle volume, and thus can be modulated by altering particle diameter, as described herein. Generally, it has been observed that large objects refract more light than smaller objects leading to high forward scatter signals (and vice versa). Accordingly, particle diameter in one embodiment is altered to modulate FSC properties of a polymer bead. For example, polymer bead diameter is increased in one embodiment is altered by harnessing larger microfluidic channels during particle formation.

SSC can be engineered by encapsulating nanoparticles within polymer bead to mimic organelles in a target cell. In some embodiments, a polymer bead of the disclosure comprises one or more types of nanoparticles selected from the group consisting of: polymethyl methacrylate (PMMA) nanoparticles, polystyrene (PS) nanoparticles, and silica nanoparticles. Without wishing to be bound by theory, the ability to selectively tune both forward and side scatter of a polymer bead, as described herein, allows for a robust platform to mimic a vast array of cell types.

### Biomarkers

In embodiments, the hydrogel or polymer particle comprises a cell surface marker, an epitope binding region of a cell surface marker, or a combination thereof.

The polymer particles of the disclosure may be of any shape, including but not limited to spherical, non-spherical, elongated, cube, cuboid, cones and cylinders. In some embodiments, a hydrogel particle of the disclosure has material modulus properties (e.g., elasticity) more closely resembling that of a target cell as compared to a polystyrene bead of the same diameter. The polymer particle of the disclosure may also mimic extracellular vesicles, viruses, virus-like particles, spheroids, organoids, or any other biological target of interest.

Hydrogel or polymer particles can be functionalized, allowing them to mimic optical properties of labeled biological particles. Functionalization can be mediated by a compound comprising a free amine group, e.g. allylamine, which can be incorporated into a hydrogel particle during the formation process. The polymer particles of the present invention may be functionalized with any biomarker, polypeptide, peptide, protein, epitope, or antigen known in the art including but not limited to: CD3, CD4, CD8, CD19, CD14, ccr7, CD45, CD45RA, CD27, CD16, CD56, CD127, CD25, CD38, HLA-DR, PD-1, CD28, CD183, CD185, CD57, IFN-gamma, CD20, TCR gamma/delta, TNF alpha, CD69, IL-2, Ki-67, CCR6. CD34, CD45RO, CD161, IgD, CD95, CD117, CD123, CD11e, IgM, CD39, FoxP3, CD10, CD40L, CD62L, CD194, CD314, IgG, TCR V alpha 7.2, CD11b, CD21, CD24, IL-4, Biotin, CCR10, CD31, CD44, CD138, CD294, NKp46, TCR V delta 2, TIGIT, CD1c, CD2, CD7, CD8a, CD15, CD32, CD103, CD107a, CD141, CD158, CD159c, TL-13, IL-21, KLRG1, TIM-3, CCR5, CD5, CD33, CD45.2, CD80, CD159a (NKG2a), CD244, CD272, CD278, CD337, Granzyme B, Ig Lambda Light Chain, IgA, IL-17A, Streptavidin, TCR V delta 1, CD1d, CD26, CD45R (B220), CD64, CD73, CD86, CD94, CD137, CD163, CD193, CTLA-4, CX3CR1, Fc epsilon R1 alpha, IL-22, Lag-3, MIP-1 beta, Perforin, TCR V gamma 9, CD1a, CD22, CD36, CD40, CD45R, CD66b, CD85j, CD160, CD172a, CD186, CD226, CD303. CLEC12A, CXCR4, Helios, Ig Kappa Light Chain, IgE, IgG1, IgG3, IL-5, IL-8, IL-21 R, KIR3dl05, KLRC1/2, Ly-6C, Ly-6G, MHC Class II (I-A/-E), MHC II TCR alpha/beta, TCR beta, TCR V alpha 24, Akt (pS473), ALDH1A1, Annexin V, Bcl-2, c-Met, CCR7, cd16/32, cd41a, CD3 epsilon, CD8b, CD11b/c, CD16/CD32, CD23, CD29, CD43, CD45.1, CD48, CD49b, CD49d, CD66, CD68, CD71, CD85k, CD93, CD99, CD106, CD122, CD133, CD134, CD146, CD150, CD158b, CD158b1/b2, j, CD158e, C-D166, CD169, CD184, CD200, CD200 R, CD235a, CD267, CD268, CD273, CD274, CD3l17, CD324, CD326, CD328, CD336, CD357, CD366, DDR2, eFluor 780 Fix Viability, EGF Receptor, EGFR (pY845), EOMES, EphA2, ERK1/2 (pT202/Y204), F4/80, FCRL5, Flt-3, FVS575V, FVS700, Granzyme A, HlER2/ErbB2, Hes1, Hoechst (33342), ICAM-1, TFN-alpha, IgA1, IgA1/IgA2, IgA2, IgG2, IgG4, IL-1 RAcP, IL-6, IL-10, IL-12, IL-17, Integrin alpha 4 beta 7, isotype Ctrl, KLRC1, KLRC2, Live/Dead Fix Aqua, Ly-6A/Ly-6E, Ly-6G/Ly-6C, Mannose Receptor, MDR1, Met (pY1234/pY1235), MMP-9, NOF Receptor p75, ORAI1, ORAI2, ORAI3, p53, P2RY12, PARP, cleaved, RT1B, S6 (pS235/pS236), STIM1, STIM2, TCR delta, TCR delta/gamma, TCR V alpha 24 J alpha 18, TCR V beta 11, TCR V gamma 1.1, TCR V gamma 2, TER-119, TIMP-3, TRAF3, TSLP Receptor, VDAC1, Vimentin, XCR1, and YAP1.

Hydrogel particles, in one embodiment, are functionalized with one or more cell surface markers (see, e.g., Tables 1, 2, and 3), or fragments thereof, for example, extracellular portions thereof in the case of transmembrane proteins, for example, by attaching the one or more cell surface markers, extracellular portions or ligand binding regions thereof to the particle via a free amine, free carboxyl and/or free hydroxyl group present on the surface of the hydrogel particle. Functionalization of a hydrogel particle with a dye or cell surface molecule can also occur through a linker, for example a streptavidin/biotin conjugate.

Depending on the target cell, individual hydrogel particles can be derivatized with one or more cell surface markers, or fragments thereof, for example, extracellular portions thereof in the case of transmembrane proteins to further mimic the structural properties of the target cell. Tables 4 and 7-8, provided below, sets forth a non-limiting list of cell surface markers that can be used to derivative hydrogel particles, depending on the target cell. Although the cell surface marker is provided, it is understood that a portion of the cell surface marker, for example, a receptor binding portion, a ligand binding portion, or an extracellular portion of the marker can be used to derivative the hydrogel particle (at the free functional group, as described above).

TABLE 1

| | Cell Surface | Cell Surface Marker(s) |
|---|---|---|
| Target Cell | Marker(s) (human) | (mouse) |
| B Cell | CD19, CD20 | CD19, CD22 (B cell |
| | | activation marker), |
| | | CD45R/B220 |
| T Cell | CD3, CD4, CD8 | CD3, CD4, CD8 |
| Activated T Cells | CD25, CD69 | CD25, CD69 |
| Dendritic Cell | CD1c, CD83, | CD11c, CD123, MHC II |
| | CD123, CD141, | |
| | CD209, MHC II | |
| Plasmacytoid | CD123, CD303, | CD11c^{int}, CD317 |
| Dendritic Cells* | CD304 | |
| Platelet (resting) | CD42b | CD41 |
| Platelet (activated) | CD62P | CD62P |
| Natural Killer | CD16, CD56 | CD49b (clone DX5) |
| Cells | | |
| Hematopoietic | CD34, CD90 | CD48, CD117, CD150, |
| Stem Cell | | Sca-1 |
| Macrophage | CD11b, CD68, | F4/80, CD68 |
| | CD163 | |
| Monocyte | CD14, CD16, CD64 | CD11b, CD115, Ly-6C |
| Plasma Cell | CD138 | CD138 |
| Red Blood Cell | CD235a | TER-119 |
| Neutrophil | CD15, CD16 | CD11b, Ly-6B.2, Ly6G, |
| | | Gr-1 |
| Basophil | 2D7 antigen, CD123, | CD200R3, FcεRIα |
| | CD203c, FcεRIα | |
| Eosinophil | CD11b, CD193, | CD11b, CD193, F4/80, |
| | EMR1, Siglec-8 | Siglec-F |
| Granulocyte | CD66b | CD66b, Gr-1/Ly6G, Ly6C |
| Endothelial cell | CD146 | CD146 MECA-32, CD106, |
| | | CD31, CD62E |
| | | (activated endothelial cell) |
| Epithelial cell | CD326 | CD326 (EPCAM1) |
| Natural Killer | CD56 | CD335 (NKp46) |
| (NK) cell | | |
| Myeloid derived | CD11b, CD14, CD33 | CD11b, GR1 |
| suppressor cell | (Siglec-3) | |
| (MDSC) | | |

TABLE 2
B Cell maturation markers for use with the hydrogel particles
described herein.

| B-cell type | Cell surface marker(s) |
|---|---|
| Pro-B | CD19, CD20, CD34, CD38, CD45R |
| Pre-B | CD19, CD20, CD38, CD45R |
| Immature B | CD19, CD20, CD40, CD45R, IgM |
| Tr-B | CD10, CD19, CD20, CD24, CD28 |
| Naïve-B | CD19, CD20, CD23, CD40, CD150 (SLAM), IgD, IgM |
| B-1 | CD19, CD20, CD27, IgM |
| Memory B | CD19, CD20, CD28, CD40, IgA, IgG |
| Plasma Cell | CD9, CD28, CD31, CD38, CD40, CD95 (FAS), CD184 |
| | (CXCR4) |

TABLE 3
Cell surface markers for use with the hydrogel particles described herein.

| | | |
|---|---|---|
| 14-3-3 Î ± Î² | C20orf30 | CD300 |
| 14-3-3 Îμ | C20orf43 | CD300a |
| 14-3-3 Î¶ | C21orf56 | CD300e |
| 14-3-3 Î, | C21orf59 | CD300f |
| 14-3-3 Ïf | C2orf43 | CD301 |
| 15-Lipoxygenase 1 | C3 | CD303 |
| 160 kD Neurofilament | C3aR | CD303a |
| Medium | | |
| 200 kD Neurofilament | C3b | CD304 |
| Heavy | | |
| 2H2 | C3c | CD305 |
| 3G11 sialoganglioside | C3d | CD307d |
| antigen | | |
| 4E-BP1 | C4 | CD309 |
| 4E-BP1 Phospho | C4 binding protein | CD31 |
| (Thr37/46) | | |
| 5-Methylcytidine | C4b | CD310 |
| 5HT3A receptor | C4c | CD312 |
| 5T4 | C4d | CD314 |
| 68 kDa Neurofilament | C4orf42 | CD314 (activating) |
| Light | | |
| 7.1 | C5 | CD314 (blocking) |
| 70 kD Neurofilament | C5aR1 | CD317 |
| Light | | |
| A20 | C5L2 | CD318 |
| A2B5 | C6 | CD319 |
| AAK1 | C6orf64 | CD32 |
| ABCA1 | C8A/B/G | CD321 |
| ABCA7 | C9 | CD323 |
| ABCB4 | C9orf41 | CD324 |
| ABCB5 | CA125 | CD325 |
| ABCC10 | CA19.9 | CD326 |
| ABCC11 | CAB39 | CD328 |
| ABCG1 | CACNA1S | CD329 |
| ABI2 | CACNA2 | CD32B |
| ABIN3 | CACNG1 | CD33 |
| ABIN3Î² | CAD | CD334 |
| ABL2 | Cadherin 1 | CD335 |
| Abraxas | Cadherin 10 | CD336 |
| ACAA1 | Cadherin 11 | CD337 |
| ACADM | Cadherin 7 | CD338 |
| ACAT2 | Cadherin 8 | CD339 |
| ACBD3 | Cadherin 9 | CD34 |
| ACD | Cadherin E | CD340 |
| ACE2 | Cadherin H | CD344 |
| Acetyl Coenzyme A | Cadherin K | CD349 |
| Carboxylase | | |
| Acetyl Coenzyme A | Cadherin P | CD35 |
| Carboxylase Î± | | |
| Acetyl Coenzyme A | Cadherin R | CD351 |
| Synthetase | | |
| Acetylated Lysine | CAK C Terminus | CD354 |
| AChRÎ± | CAK N Terminus | CD357 |
| AChRÎ² | CAK Phospho | CD358 |
| | (Ser164/Thr170) | |
| AChRÎ³ | Calbindin | CD36 |
| Aconitase2 | Calcineurin A | CD360 |
| ACOT12 | Calcitonin Receptor | CD361 |
| ACSA2 | Calcium Sensing | CD36L1 |
| | Receptor | |
| ACSF2 | Caldesmon | CD37 |
| ACSM5 | Calgranulin A | CD38 |
| Act1 | Calgranulin B | CD39 |
| Activation molecule 8 | Calmodulin | CD39L4 |
| (B cells) | | |
| Activin A Receptor | Calnexin - ER | CD3D |
| Type IB | membrane marker | |
| Activin A Receptor | Calpain 1 | CD3G |
| Type IIB | | |
| ACTN3 | Calpain 2 | CD3Î³ |
| ACY1 | Calpain 9 | CD3Î\acute{ } |
| ACY3 | Calpain S1 | CD3Îμ |
| | (small subunit) | |
| ADA | Calpastatin | CD3Îμ (CD3 |
| | | Molecular Complex) |
| ADAM12 | Calponin | CD4 |
| ADE2 | Calreticulin | CD4 (domain 1) |
| Adenosine A1 Receptor | Calretinin | CD4 (domain 2) |
| Adenosine A2aR | Calsequestrin 2 | CD4 v4 |
| Adenovirus | CaMKI | CD40 |
| Adenovirus Fiber | CaMKII | CD40bp |
| monomer and trimer | | |
| Adenovirus hexon protein | CaMKII Phospho | CD41 |
| | (Thr286) | |
| Adenylate Kinase 1 | CaMKIIÎ\acute{ } | CD41/CD61 |
| Adenylosuccinate Lyase | CamKIV | CD41a |
| ADFP | CaMKIÎ± | CD41b |
| ADH1B | CAMLG | CD42a |
| ADH6 | CAMP Protein Kinase | CD42b |
| | Catalytic subunit | |
| ADH7 | CAMP Protein Kinase | CD42d |
| | Catalytic subunit Î± | |
| ADI1 | Cannabinoid | CD43 |
| | Receptor I | |
| Adiponectin | Cannabinoid | CD44 |
| | Receptor II | |
| Adiponectin Receptor 2 | CAP-G2 | CD44 (v3) |
| Adipose Triglyceride | CAP18 | CD44 (v4) |
| Lipase | | |
| ADP Ribosylation Factor | CAP2 | CD44 (v5) |
| ADP-ribosyltransferase | CAP3 | CD44 (v6) |
| 2.2 gene | | |
| Adrenodoxin | Carbonic Anhydrase I | CD44 (v7) |
| AF10 | Carbonic Anhydrase | CD44.2 |
| | IX | |
| AFAP1 | Carboxylesterase 1 | CD44std |
| AFP | Carboxypeptidase A1 | CD44v6 |
| AG2 | Carboxypeptidase A2 | CD44var (v10) |
| AGAP1 | CARD11 | CD44var (v3) |
| AGPAT5 | CARD8 | CD44var (v3-v10) |
| AGR2 | CARD9 | CD44var (v4) |
| AHSG | Cardiac Troponin T | CD44var (v5) |
| AICDA | CARKL | CD44var (v6) |
| AID | CARM1 | CD44var (v7) |
| AIF | Casein Kinase 1 Î± | CD44var (v7-v8) |
| AIM-2 | Casein Kinase 1 Î³2 | CD45 |
| Aiolos | Casein Kinase 2 Î² | CD45.1 |
| AIPL1 | Caspase 1 | CD45.2 |
| AIRE | Caspase 10 | CD45R |
| AK3 | Caspase 11 | CD45RA |
| AK3L1 | Caspase 12 | CD45RB |
| AK5 | Caspase 2 | CD45RC |
| Akt | Caspase 2L | CD45RO |
| Akt (pS473) | Caspase 3 | CD46 |
| Akt (pT308) | Caspase 4 | CD47 |
| Akt1 | Caspase 5 | CD48 |
| Akt2 | Caspase 6 | CD49a |
| Akt3 | Caspase 7 | CD49a/CD29 |
| Albumin | Caspase 8 | CD49b |
| Alcohol Dehydrogenase | Caspase 9 | CD49b/CD29 |
| Aldehyde Reductase | Catalase | CD49b/CD61 |
| ALDH1A1 | Catechol-O- | CD49c |
| | methyltransferase | |
| ALDH1L1 | Cathepsin D | CD49d |
| ALDH2 | Cathepsin K | CD49d/CD29 |
| ALDH3A1 | Cathepsin L | CD49e |
| ALDH3A2 | Caveolin1 | CD49e/CD29 |
| ALDH5A1 | Caveolin1 (pY14) | CD49f |
| ALDH6A1 | Caveolin2 | CD49f/CD29 |
| ALDH7A1 | Cbl | CD4IÎ± |
| ALDOB | CBP | CD5 |
| Aldolase B | CBWD1 | CD5.1 |
| Alexa Fluor 405/Cascade | CBX1 | CD5.2 |
| Blue | | |
| Alexa Fluor 488 | cCbl (pY700) | CD5.6 |
| ALG2 | cCbl (pY774) | CD50 |
| Alix | CCDC98 | CD51 |
| Allergin 1 | CCK4 | CD51/61 |
| alpha 1 Antitrypsin | CCL11 | CD52 |
| alpha 1 Catenin | CCL17 | CD53 |
| alpha 1 Sodium Potassium | CCL18 | CD54 |
| ATPase | | |
| alpha 2 Catenin | CCL19-Fc | CD55 |
| alpha 2 Macroglobulin | CCL20 | CD56 |
| alpha Actin 1 | CCL21 | CD57 |
| alpha Actin 2 | CCL25 | CD58 |
| alpha Actinin | CCL3 | CD59 |
| alpha Actinin 2 | CCL5 | CD59a |
| alpha Actinin 3 | CCL6 | CD6 |
| alpha Actinin 4 | CCNB1IP1 | CD60b |
| alpha Adaptin | CCR10 | CD61 |
| alpha Adducin | CCR11 | CD62E |
| alpha B Crystallin | CCRD6 | CD62L |
| alpha Fodrin | CCRL2 | CD62P |
| alpha Internexin | CD1 | CD63 |
| alpha Synuclein | CD1.1 | CD64 |
| ALS1 | CD10 | CD64 a,b alloantigens |
| AMACR | CD100 | CD64.1 |
| Aminopeptidase P | CD101 | CD65 |
| AML1 | CD102 | CD65s |
| | | (CD65 sialylated) |
| Amphiphysin | CD103 | CD66 |
| AMPKÎ± | CD104 | CD66a |
| AMPKÎ±1 | CD105 | CD66a/b/c/e |
| AMPKÎ±2 | CD106 | CD66a/c/d |
| AMPKÎ²1 | CD107a | CD66a/c/d/e |
| AMPKÎ³1 | CD107b | CD66a/c/e |
| AmyloidÎ² 42 | CD108 | CD66a/e |
| ANAPC2 | CD109 | CD66b |
| AND1 | CD11 | CD66c |
| Androgen Receptor | CD110 | CD66c/e |
| Angiotensin I | CD111 | CD66e |
| Angiotensin II Receptor 2 | CD112 | CD66f |
| Angiotensin III | CD113 | CD68 |
| ANKRD53 | CD114 | CD69 |
| Annexin IV | CD115 | CD7 |
| Annexin V | CD116 | CD70 |
| ANP | CD117 | CD70b |
| Anti-Kudoa thrysites | CD118 | CD71 |
| Anti-T. brucei procyclin | CD119 | CD72 |
| (GPEET) | | |
| Anti-T. brucei procyclin | CD11a | CD72 a,b,c |
| (phosphorylated GPEET) | | alloantigens |
| Antiglobulin (Coombs) | CD11a, strain | CD72 b,c alloantigens |
| | polymorphism | |
| Antithrombin III | CD11a/CD18 | CD72.1 |
| AP2 Î± | CD11b | CD73 |
| AP2 Î±Î² | CD11b/c | CD74 |
| AP2 Î³ | CD11c | CD75 |
| AP2M1 | CD11d | CD77 |
| AP2S1 | CD120a | CD78 |
| APAF1 | CD120b | CD79a |
| APBB3 | CD121a | CD79b |
| APC | CD121b | CD8 |
| APC-1 | CD122 | CD80 |
| APC-10 | CD123 | CD81 |
| APC-11 | CD124 | CD82 |
| APC-2 | CD125 | CD83 |
| APC-3 | CD126 | CD84 |
| APC-5 | CD127 | CD85 |
| APC-7 | CD129 | CD85a |
| APC-8 | CD13 | CD85d |
| APE1 | CD130 | CD85g |
| APG12 | CD131 | CD85h |
| APG3 | CD132 | CD85j |
| APG5 | CD133 | CD85k |
| APG7 | CD133/2 | CD86 |
| APMAP | CD134 | CD87 |
| Apo-2.7 | CD135 | CD88 |
| Apo-2.7 (7A6) | CD136 | CD89 |
| ApoE | CD137 | CD8Î± |
| ApoE4 | CD137L | CD8Î±.1 |
| APOER2 | CD138 | CD8Î±.2 |
| Apolipoprotein AI | CD139 | CD8Î² |
| Apolipoprotein AII | CD14 | CD9 |
| Apolipoprotein AIV | CD140a | CD90.1 |
| Apolipoprotein B | CD140b | CD90.2 |
| Apolipoprotein CIII | CD140b (pY1009) | CD90.9 |
| Apolipoprotein D | CD140b (pY1021) | CD91 |
| Apolipoprotein E | CD140b (pY771) | CD91Î± |
| Apolipoprotein F | CD140b (pY857) | CD91Î² |
| Apolipoprotein H | CD141 | CD93 |
| Apolipoprotein J | CD142 | CD94 |
| Apolipoprotein L1 | CD143 | CD95 |
| Apolipoprotein M | CD144 | CD96 |
| Apoptotic neutrophils | CD146 | CD97 |
| APP | CD147 | CD98 |
| Aquaporin 1 | CD148 | CD98hc |
| Aquaporin 5 | CD15 | CD99 |
| ARF1 | CD150 | CD99R |
| ARF5 | CD151 | |
| ARFGAP1 | CD152 | |
| ARFRP1 | CD153 | |
| Argonaute-1 | CD154 | |
| ARH | CD155 | |
| ARHGAP25 | CD156c | |
| ARHGAP4 | CD157 | |
| ARL11 | CD158a | |
| ARL5B | CD158a/h | |
| ARPC5 | CD158b | |
| Artemis | CD158b1/b2/j | |
| Aryl hydrocarbon | CD158d | |
| Receptor | | |
| ASB-1 | CD158e | |
| ASCC1 | CD158e/k | |
| ASCC2 | CD158e1 | |
| ASGPR | CD158e1/e2 | |
| Asialo-GM1 | CD158f | |
| ASK1 | CD158g | |
| Asparagine synthetase | CD158h | |
| Ataxin 1 | CD158i | |
| ATF1 | CD158j | |
| ATF2 | CD159a | |
| ATG4A | CD159c | |
| ATG9A | CD15s | |
| ATIC | CD16 | |
| Atlantic Salmon Ig | CD16/32 | |
| ATM | CD16/56 | |
| ATP citrate lyase | CD160 | |
| ATP1B3 | CD161 | |
| ATP5A | CD161a | |
| ATP5H | CD162 | |
| ATP5J | CD162R | |
| ATP50 | CD163 | |
| ATP6VOD1 | CD164 | |
| ATP6V1B1 | CD165 | |
| ATPB | CD166 | |
| ATRIP | CD167a | |
| Aurora A | CD168 | |
| Aurora A Phospho | CD169 | |
| (Thr288) | | |
| Aurora B | CD16b | |
| Aurora B Phospho | CD17 | |
| (Thr232) | | |
| AVEN | CD170 | |
| Avian Influenza A | CD171 | |
| Neuraminidase | | |
| Avidin | CD172 | |
| Axin 2 | CD172a | |
| Axl | CD172a/b | |
| B and Activated T Cells | CD172b | |
| B Cell | CD172g | |
| B Cell Subset | CD173 | |
| B cells (pan reactive) | CD177 | |
| B lymphocytes antibody | CD178 | |
| [UCH-B1] | | |
| b-Endorphin | CD178.1 | |
| B-Raf Phospho | CD179a | |
| (Thr598/Ser601) | | |
| B18R | CD179b | |
| B7-H4 | CD18 | |
| BACE1 | CD180 | |
| BACE2 | CD181 | |
| BACH1 | CD182 | |
| baculovirus envelope | CD183 | |
| gp64 protein | | |
| BAG1 | CD184 | |
| BAG2 | CD185 | |
| BAG3 | CD186 | |
| BAG4 | CD19 | |
| BAIAP2 | CD191 | |
| BAK | CD192 | |
| BAMBI | CD193 | |
| BAP31 | CD194 | |
| BAP37 | CD195 | |
| basal cell Cytokeratin | CD195 (cytoplasmic) | |
| Basophils | CD195 Phospho | |
| | (Ser337) | |
| Bassoon | CD195 Phospho | |
| | (Ser349) | |
| BATF | CD196 | |
| Bax | CD197 | |
| BCAR1 | CD198 | |
| BCAR2 | CD199 | |
| BCKD complex E2 | CD1a | |
| subunit | | |
| Bcl-10 | CD1b | |
| Bcl-2 | CD1b/c | |
| Bcl-2 (pS70) | CD1c | |
| Bcl-2 like 12 | CD1d | |
| Bcl-2 like 2 | CD1d Î± GalCer | |
| | Complex | |
| Bcl-22 | CD2 | |
| Bcl-2A1 | CD20 | |
| Bcl-2Î± | CD200 | |
| Bcl-3 | CD200R | |
| Bcl-6 | CD200R3 | |
| Bcl-xL | CD201 | |
| Bcl-XS/L | CD202b | |
| BCR | CD203a | |
| BCSC1 | CD203c | |
| BDH2 | CD204 | |
| BDKRB2 | CD205 | |
| BDNF | CD206 | |
| Beclin 1 | CD207 | |
| Bestrophin 3 | CD208 | |
| beta 2 Adrenoreceptor | CD209 | |
| Beta 3 Adrenergic | CD209b | |
| Receptor | | |
| beta 3 Sodium Potassium | CD21 | |
| ATPase | | |
| beta Actin | CD21/CD35 | |
| beta Arrestin 1 | CD210 | |
| beta Arrestin 2 | CD212 | |
| beta Catenin | CD213a1 | |
| beta Catenin (npaa 27-37) | CD213a2 | |
| beta Catenin (npaa 35-50) | CD217 | |
| beta Catenin (pS45) | CD218a | |
| beta Dystroglycan | CD22 | |
| beta galactosidase | CD22 (pY822) | |
| beta galactosidase fusion | CD22.2 | |
| proteins | | |
| beta Synuclein | CD220 | |
| beta2 Microglobulin | CD220Î± | |
| BHMT | CD221 | |
| Bid | CD221 (pY1131) | |
| Biglycan | CD222 | |
| Bilirubin Oxidase | CD223 | |
| Bim | CD224 | |
| BimL | CD226 | |
| BIN1 | CD227 | |
| BIN3 | CD229 | |
| Biotin | CD229.1 | |
| BiP | CD23 | |
| BLBP | CD230 | |
| Blimp-1 | CD231 | |
| BLK | CD233 | |
| BLNK | CD234 | |
| BLNK (pY84) | CD235a | |
| Blood Group A Antigen | CD235ab | |
| Blood Group AB Antigen | CD236 | |
| Blood Group B Antigen | CD239 | |
| Blood Group H ab | CD24 | |
| Antigen | | |
| Blood Group H ab | CD240CE | |
| Antigen/n Antigen | | |
| Blood Group H inhibitor | CD240DCE | |
| Blood Group Lewis a | CD243 | |
| Blood Group M Antigen | CD244 | |
| Blood Group N Antigen | CD244.1 | |
| Blooms Syndrome Protein | CD244.2 | |
| Blm | | |
| BM1 | CD245 | |
| BMAL1 | CD246 | |
| BMI1 | CD247 | |
| Bmk | CD247 (pY142) | |
| BMP15 | CD249 | |
| BMP4 | CD25 | |
| BMP7 | CD252 | |
| BMPR1A | CD253 | |
| BMPR2 | CD254 | |
| BMX | CD255 | |
| bMyc | CD256 | |
| BNIP2 | CD257 | |
| BNIP3 | CD258 | |
| BNIP3L | CD26 | |
| BOB1 | CD261 | |
| BORA | CD262 | |
| Borealin | CD263 | |
| Borrelia burgdorferi | CD264 | |
| BPI | CD265 | |
| BRaf | CD266 | |
| BRCA1 | CD267 | |
| BRCC36 | CD268 | |
| BRD3 | CD269 | |
| BrdU | CD27 | |
| BRF1 | CD270 | |
| BRG1 | CD271 | |
| BRN3A | CD272 | |
| Btk | CD273 | |
| Btk (pY551)/Itk (pY511) | CD274 | |
| BTLN-2 | CD275 | |
| BTN1A1 | CD276 | |
| Bu1 | CD277 | |
| Bu1a | CD278 | |
| Bu1a/Bu1b | CD279 | |
| Bu1b | CD28 | |
| BubR1 | CD280 | |
| Bulb | CD281 | |
| Butyrylcholinesterase | CD282 | |
| C peptide | CD283 | |
| C reactive protein | CD284 | |
| C/EBPÎ² | CD284/MD2 | |
| | Complex | |
| C1 Inhibitor | CD286 | |
| C15orf40 | CD289 | |
| C16orf72 | CD29 | |
| C1orf50 | CD290 | |
| C1Q | CD294 | |
| C1QA | CD298 | |
| C1QB | CD299 | |
| C1QC | CD2a | |
| C1QG | CD3 | |
| C1r | CD3/CD44 | |
| C1s | CD30 | |

Hydrogels and other polymer particles are known in the art and are described in U.S. Pat. Nos. 9,915,598 and 10,753,846, incorporated herein by reference in their entirety.

### Fluorophores

The present invention may use any fluorophore known in the art, including fluorescent dyes, tags and stains as listed in The MolecularProbes® Handbook—A Guide to Fluorescent Probes and Labeling Technologies, incorporated herein by reference in its entirety. Tags include surface enhanced raman scattering (SERS) tags. In embodiments, the hydrogel or polymer particles can be functionalized with fluorophores by covalent interactions, non-covalent interactions, or a combination thereof. In embodiments, the hydrogel or polymer particles can be functionalized with the fluorophores, either through biomarker or antibody mediation or by direct conjugation via, e.g., amine-reactive fluorophores. Similar to the above, functionalization can be facilitated by a free amine group, such as allylamine, which can be incorporated into a hydrogel particle during the formation process.

In embodiments, the fluorophore, or fluorescent dye, is selected from one or more of: peridinin chlorophyll protein-cyanine 5.5 dye (PerCP-Cy5.5); phycoerythrin-cyanine7 (PE Cy7); allophycocyanin-cyanine 7 (APC-Cy7); fluorescein isothiocyanate (FITC); phycoerythrin (PE); allophyscocyanin (APC); 6-carboxy-4′,5′-dichloro-2′,7′-dimethoxyfluorescein succinimidylester; 5-(and-6)-carboxyeosin; 5-carboxyfluorescein; 6 carboxyfluorescein; 5-(and-6)-carboxyfluorescein; S-carboxyfluorescein-bis-(5-carboxymethoxy-2-nitrobenzyl)ether,-alanine-carboxamide, or succinimidyl ester; 5-carboxy fluorescein succinimidyl ester; 6-carboxyfluorescein succinimidyl ester; 5-(and-6)-carboxyfluorescein succinimidyl ester; 5-(4,6-dichlorotriazinyl)amino fluorescein; 2′,7′-difluoro fluorescein; eosin-5-isothiocyanate; erythrosin5-isothiocyanate; 6-(fluorescein-5-carboxamido) hexanoic acid or succinimidyl ester; 6-(fluorescein-5-(and-6)-carboxamido) hexanoic acid or succinimidylester; fluorescein-S-EX succinimidyl ester; fluorescein-5-isothiocyanate; fluorescein-6-isothiocyanate; OregonGreen® 488 carboxylic acid, or succinimidyl ester; Oregon Green® 488 isothiocyanate; Oregon Green® 488-X succinimidyl ester; Oregon Green® 500 carboxylic acid; Oregon Green® 500 carboxylic acid, succinimidylester or triethylammonium salt; Oregon Green® 514 carboxylic acid; Oregon Green® 514 carboxylic acid or succinimidyl ester; RhodamineGreen™ carboxylic acid, succinimidyl ester or hydrochloride; Rhodamine Green™ carboxylic acid, trifluoroacetamide or succinimidylester; Rhodamine Green™-X succinimidyl ester or hydrochloride; RhodolGreen™ carboxylic acid, N,O-bis-(trifluoroacetyl) or succinimidylester; bis-(4-carboxypiperidinyl) sulfonerhodamine or di(succinimidylester); 5-(and-6)carboxynaphtho fluorescein, 5-(and-6)carboxynaphthofluorescein succinimidyl ester; 5-carboxyrhodamine 6G hydrochloride; 6-carboxyrhodamine6Ghydrochloride, 5-carboxyrhodamine 6G succinimidyl ester; 6-carboxyrhodamine 6G succinimidyl ester; 5-(and-6)-carboxyrhodamine6G succinimidyl ester; 5-carboxy-2′,4′,5′,7′-tetrabromosulfonefluorescein succinimidyl esteror bis-(diisopropylethylammonium) salt; 5-carboxytetramethylrhodamine; 6-carboxytetramethylrhodamine; 5-(and-6)-carboxytetramethylrhodamine; 5-carboxytetramethylrhodamine succinimidyl ester; 6-carboxytetramethylrhodaminesuccinimidyl ester; 5-(and-6)-carboxytetramethylrhodamine succinimidyl ester; 6-carboxy-X-rhodamine; 5-carboxy-X-rhodamine succinimidyl ester; 6-carboxy-Xrhodamine succinimidyl ester; 5-(and-6)-carboxy-Xrhodaminesuccinimidyl ester; 5-carboxy-X-rhodamine triethylammonium salt; Lissamine™ rhodamine B sulfonyl chloride; malachite green; isothiocyanate; NANOGOLD® mono(sulfosuccinimidyl ester); QSY® 21carboxylic acid or succinimidyl ester; QSY® 7 carboxylic acid or succinimidyl ester; Rhodamine Red™-X succinimidyl ester; 6-(tetramethylrhodamine-5-(and-6)-carboxamido) hexanoic acid; succinimidyl ester; tetramethylrhodamine-5-isothiocyanate; tetramethylrhodamine-6-isothiocyanate; tetramethylrhodamine-5-(and-6)-isothiocyanate; Texas Red® sulfonyl; Texas Red® sulfonyl chloride; Texas Red®-X STP ester or sodium salt; Texas Red®-X succinimidyl ester; Texas Red®-X succinimidyl ester; andX-rhodamine-5-(and-6) isothiocyanate, BODIPY® dyes commercially available from Invitrogen, including, but not limited to BODIPY® FL; BODIPY® TMR STP ester; BODIPY® TR-X STP ester; BODIPY® 630/650-X STPester; BODIPY® 650/665-X STP ester; 6-dibromo-4,4-difluoro-5,7-dimethyl-4-bora-3a,4a-diaza-s-indacene-3-propionic acid succinimidyl ester; 4,4-difluoro-4-bora-3a,4a-diaza-s-indacene-3,5-dipropionic acid; 4,4-difluoro-5,7-dimethyl-4-bora-3a,4a-diaza-s-indacene-3-pentanoicacid; 4,4-difluoro-5,7-dimethyl-4-bora3a,4a-diaza-s-indacene-3-pentanoicacid succinimidyl ester; 4,4-difluoro-5,7-dimefhyl-4-bora-3a,4a-diaza-s-indacene-3propionicacid; 4,4-difluoro-5,7-dimethyl-4-bora-3a,4adiaza-s-indacene-3-propionicacid succinimidyl ester; 4,4difluoro-5,7-dimethyl-4-bora-3a,4a-diaza-s-indacene-3propionic acid; sulfosuccinimidyl ester or sodium salt; 6-((4,4-difluoro-5,7-dimethyl-4-bora-3a,4a-diaza-s-indacene-3propionyl)amino)hexanoicacid; 6-((4,4-difluoro-5,7 dimethyl-4-bora-3a,4a-diaza-s-indacene-3-propionyl)amino)hexanoic acid or succinimidyl ester; N-(4,4-difluoro 5,7-dimethyl-4-bora-3a,4a-diaza-s-indacene-3-propionyl) cysteic acid, succinimidyl ester or triethylammonium salt; 6-4,4-difluoro-1,3-dimethyl-5-(4-methoxyphenyl)-4-bora3a,4a4,4-difluoro-5,7-diphenyl-4-bora-3a,4a-diaza-sindacene-3-propionicacid; 4,4-difluoro-5,7-diphenyl-4-bora3a,4a-diaza-s-indacene-3-propionicacid succinimidyl ester; 4,4-difluoro-5-phenyl-4-bora-3a,4a-diaza-s-indacene-3-propionic acid; succinimidyl ester; 6-((4,4-difluoro-5-phenyl-4 bora-3a,4a-diaza-s-indacene-3-propionyl)amino) hexanoicacid or succinimidyl ester; 4,4-difluoro-5-(4-phenyl-1,3butadienyl)-4-bora-3a,4a-diaza-s-indacene-3-propionicacid succinimidyl ester; 4,4-difluoro-5-(2-pyrrolyl)-4-bora-3a,4a-diaza-s-indacene-3-propionic acid succinimidyl ester; 6-(((4,4-difluoro-5-(2-pyrrolyl)-4-bora-3a,4a-diaza-s-indacene-3-yl)styryloxy)acetyl)aminohexanoicacid or succinimidyl ester; 4,4-difluoro-5-styryl-4-bora-3a, 4a-diaza-s-indacene-3-propionic acid; 4,4-difluoro-5-styryl-4-bora-3a,4a-diaza-sindacene-3-propionic acid; succinimidyl ester; 4,4-difluoro-1,3,5,7-tetramethyl-4-bora-3a,4adiaza-s-indacene-8-propionicacid; 4,4-difluoro-1,3,5,7-tetramethyl-4bora-3a,4a-diaza-sindacene-8-propionic acid succinimidyl ester; 4,4-difluoro-5-(2-thienyl)-4-bora-3a,4a-diaza-sindacene-3-propionic acid succinimidyl ester; 6-(((4-(4,4-difluoro-5-(2-thienyl)-4-bora-3a,4adiazas-indacene-3-yl)phenoxy)acetyl)amino)hexanoic acid or succinimidyl ester; and 6-(((4,4-difluoro-5-(2-thienyl)-4-bora-3a,4a-diaza-s-indacene-3-yl)styryloxy)acetyl)aminohexanoic acid or succinimidyl ester, Alexa fluor dyes commercially available from Invitrogen, including but not limited to Alexa Fluor®350 carboxylic acid; Alexa Fluor® 430 carboxylic acid; Alexa Fluor® 488 carboxylic acid; Alexa Fluor®532 carboxylic acid; Alexa Fluor®546 carboxylic acid; Alexa Fluor®555 carboxylic acid; Alexa Fluor®568 carboxylic acid; Alexa Fluor®594 carboxylic acid; Alexa Fluor®633 carboxylic acid; Alexa Fluor®647 carboxylic acid; Alexa Fluor® 660 carboxylic acid; and Alexa Fluor®680 carboxylic acid, cyanine dyes commercially available from Amersham-Pharmacia Biotech, including, but not limited to Cy3 NHS ester; Cy 5 NHS ester; Cy5.5 NHSester; Cy7 NHS ester, and/or any conjugation or derivative thereof.

In embodiments, a hydrogel or polymer particle may comprise from 1 to about 20 fluorescent dyes, from 1 to about 10 fluorescent dyes, or from 1 to about 5 fluorescent dyes. In embodiments, a hydrogel or polymer particle comprises 1, 2, 3, 4, 5, 6, 7, 8, 9, about 10, about 11, about 12, about 13, about 14, about 15, about 16, about 17, about 18, about 19, or about 20 fluorescent dyes, including all values and subranges in between inclusive of endpoints.

In embodiments, a hydrogel or polymer particle comprises a “rainbow particle.” Rainbow particles contain a mixture of fluorophores. In embodiments, the rainbow particle comprises from 1 to about 20 fluorophores, from 1 to about 10 fluorophores, or from 1 to about 5 fluorophores. In embodiments, a hydrogel or polymer particle comprises a rainbow particle with 1, 2, 3, 4, 5, 6, 7, 8, 9, about 10, about 11, about 12, about 13, about 14, about 15, about 16, about 17, about 18, about 19, or about 20 fluorophores, including all values and subranges in between inclusive of endpoints. In embodiments, a user selects a wavelength with which to excite the rainbow particle with, depending on the fluorophore being interrogated. Rainbow particles are commercially available, for example, from BD Biosciences (catalog nos. 556298 (mid range FL1 fluorescence), 556286 (6 color, 3.0-3.4 μm), 556288 (6 color, 6.0-6.4 μm), 559123 (8 color)) and Spherotech in various diameters (e.g., catalog nos. RCP20-5 (4 color), RCP-30-5 (6 peaks), RCP-30-5A (8 peaks).

A non-exhaustive listing of fluorophores amenable for use with the present invention are provided in Table 4 below.

TABLE 4

| ID | NAME | Alternate Names | Excitation | Emission | Vendor/Source | ACS CAS# |
|---|---|---|---|---|---|---|
| ISAC148 | 6-carboxyfluorescein | | 492 | 518 | PubChem | 3301-79-9 |
| ISAC1 | 6-JOE | | 520 | 550 | LifeTechnologies | 82855-40-1 |
| ISAC2 | 7-AAD | | 545 | 647 | LifeTechnologies | 7240-37-1 |
| ISAC3 | Acridine Orange | | 503 | 525 | LifeTechnologies | 65-61-2 |
| ISAC4 | Alexa Fluor 350 | AF350; 2H-1-Benzopyran-6- | 343 | 442 | LifeTechnologies | 244636-14-4 |
| | | sulfonic acid, 7-amino-3-[2-[(2,5- | | | | |
| | | dioxo-1-pyrrolidinyl)oxy]-2- | | | | |
| | | oxoethyl]-4-methyl-2-oxo-; | | | | |
| | | 200554-19-4 | | | | |
| ISAC6 | Alexa Fluor 405 | AF405; C46H69N5O15S3 | 401 | 425 | LifeTechnologies | 791637-08-6 |
| ISAC7 | Alexa Fluor 430 | AF430; C32H42F3N3O9S | 433 | 541 | LifeTechnologies | 467233-94-9 |
| ISAC8 | Alexa Fluor 488 | AF488; C25H15Li2N3O13S2 | 496 | 519 | LifeTechnologies | 247144-99-6 |
| ISAC9 | Alexa Fluor 500 | AF500; CAS#798557-08-1 | 503 | 525 | LifeTechnologies | 798557-08-1 |
| ISAC10 | Alexa Fluor 514 | AF514; C31H27N3O13S2 | 517 | 542 | LifeTechnologies | 798557-07-0 |
| ISAC11 | Alexa Fluor 532 | AF532; 1H- Pyrano[3,2-f:5,6- | 532 | 553 | LifeTechnologies | 222159-92-4 |
| | | f′]diindole-10,12-disulfonic acid, | | | | |
| | | 5-[4-[[(2,5-dioxo-1- | | | | |
| | | pyrrolidinyl)oxy]carbonyl]phenyl]- | | | | |
| | | 2,3,7,8-tetrahydro-2,3,3,7,7,8- | | | | |
| | | hexamethyl-; 271795-14-3 | | | | |
| ISAC13 | Alexa Fluor 546 | AF546; C50H62Cl3N5O14S3 | 556 | 573 | LifeTechnologies | 247145-23-9 |
| ISAC14 | Alexa Fluor 555 | AF555 | 555 | 565 | LifeTechnologies | 644990-77-2 |
| ISAC15 | Alexa Fluor 568 | AF568 | 578 | 603 | LifeTechnologies | 247145-38-6 |
| ISAC16 | Alexa Fluor 594 | AF594 | 590 | 617 | LifeTechnologies | 247145-86-4 |
| ISAC17 | Alexa Fluor 610 | AF610; C58H77Cl3N6O14S3 | 612 | 628 | LifeTechnologies | 900528-62-3 |
| ISAC18 | Alexa Fluor 633 | AF633 | 632 | 647 | LifeTechnologies | 477780-06-6 |
| ISAC19 | Alexa Fluor 635 | AF635 | 633 | 647 | LifeTechnologies | 945850-82-8 |
| ISAC20 | Alexa Fluor 647 | AF647 | 650 | 665 | LifeTechnologies | 400051-23-2 |
| ISAC21 | Alexa Fluor 660 | AF660 | 663 | 690 | LifeTechnologies | 422309-89-5 |
| ISAC22 | Alexa Fluor 680 | AF680 | 679 | 702 | LifeTechnologies | 422309-67-9 |
| ISAC23 | Alexa Fluor 700 | AF700 | 702 | 723 | LifeTechnologies | 697795-05-4 |
| ISAC24 | Alexa Fluor 750 | AF750 | 749 | 775 | LifeTechnologies | 697795-06-5 |
| ISAC25 | Alexa Fluor 790 | AF790 | 784 | 814 | LifeTechnologies | 950891-33-5 |
| ISAC26 | AMCA | | 346 | 448 | SantaCruzBiotech | 106562-32-7 |
| ISAC27 | AmCyan | | 457 | 489 | BDBioscences | 1216872-44-4 |
| ISAC28 | APC | Allophycocyanin | 650 | 660 | SigmaAldrich | No names |
| | | | | | | found |
| ISAC29 | APC-Alexa Fluor 680 | APC-AF680 | 655 | 704 | LifeTechnologies | No names |
| | | | | | | found |
| ISAC30 | APC-Alexa Fluor 700 | APC-AF700 | 655 | 718 | LifeTechnologies | No names |
| | | | | | | found |
| ISAC31 | APC-Alexa Fluor 750 | APC-AF750 | 650 | 775 | LifeTechnologies | No names |
| | | | | | | found |
| ISAC32 | APC-Cy5.5 | Allophycocyanin-Cy5.5 | 650 | 695 | LifeTechnologies | No names |
| | | | | | | found |
| ISAC33 | APC-Cy7 | Allophycocyanin-Cy7 | 650 | 767 | LifeTechnologies | No names |
| | | | | | | found |
| ISAC34 | APC-eFluor 750 | eFluor750APC | 650 | 750 | eBioscience | No names |
| | | | | | | found |
| ISAC35 | APC-eFluor 780 | eFluor780APC | 650 | 780 | eBioscience | 1472056-77-1 |
| ISAC36 | APC-H7 | H7APC | 650 | 765 | BDBioscences | 1366000-62-5 |
| ISAC37 | APC-Vio770 | Vio770APC | 652 | 775 | Miltenyl Biotech | No names |
| | | | | | | found |
| ISAC38 | Atto488 | | 501 | 523 | ATTO-TEC | 923585-42-6 |
| ISAC39 | BIOTIN | | 0 | 0 | PubChem | 58-85-5 |
| ISAC40 | BODIPY FL | | 502 | 511 | SantaCruzBiotech | 165599-63-3 |
| ISAC41 | BODIPY R6G | 4,4-difluoro-5-phenyl-4-bora- | 527 | 547 | LifeTechnologies | 335193-70-9 |
| | | 3a,4a-diaza-s-indacene-3- | | | | |
| | | propionic acid, succinimidyl | | | | |
| | | ester; C22H18BF2N3O4 | | | | |
| ISAC43 | Brilliant Violet 421 | BV421 | 406 | 423 | Biolegend | 1428441-68-2 |
| ISAC44 | Brilliant Violet 510 | BV510 | 405 | 510 | Biolegend | No names |
| | | | | | | found |
| ISAC45 | Brilliant Violet 570 | BV570 | 407 | 571 | Biolegend | 1428441-76-2 |
| ISAC46 | Brilliant Violet 605 | BV605 | 407 | 603 | Biolegend | 1632128-60-9 |
| ISAC47 | Brilliant Violet 612 | BV612 | 0 | 0 | Biolegend | 1428441-91-1 |
| ISAC48 | Brilliant Violet 650 | BV650 | 407 | 647 | Biolegend | No names |
| | | | | | | found |
| ISAC49 | Brilliant Violet 711 | BV711 | 405 | 711 | Biolegend | No names |
| | | | | | | found |
| ISAC50 | Brilliant Violet 785 | BV785 | 405 | 786 | Biolegend | 1613592-44-1 |
| ISAC53 | Calcein | CAS#: 1461-15-0 | 493 | 514 | LifeTechnologies | 1461-15-0 |
| ISAC51 | Calcein AM | | 496 | 517 | PubChem | 148504-34-1 |
| ISAC52 | Calcein Blue AM | | 360 | 445 | PubChem | 168482-84-6 |
| ISAC54 | Calcein Violet AM | | 400 | 452 | LifeTechnologies | No names |
| | | | | | | found |
| ISAC55 | Calcium Sensor Dye | | 490 | 514 | eBioscience | No names |
| | eFluor 514 | | | | | found |
| ISAC56 | Cascade Blue | | 401 | 420 | PubChem | 1325-87-7 |
| ISAC57 | Cascade Yellow | | 400 | 550 | Synchem UG & | 220930-95-0 |
| | | | | | Co, KG | |
| ISAC58 | Cell Proliferation Dye | | 405 | 445 | eBioscience | No names |
| | eFluor 450 | | | | | found |
| ISAC59 | Cell Proliferation Dye | | 652 | 672 | eBioscience | No names |
| | eFluor 670 | | | | | found |
| ISAC60 | CellTrace Violet Cell | | 392 | 455 | LifeTechnologies | No names |
| | Proliferation | | | | | found |
| ISAC61 | CellVue Claret | | 655 | 657 | SigmaAldrich | 1042142-46-0 |
| ISAC62 | CFSE | | 492 | 525 | SantaCruzBiotech | 150347-59-4 |
| ISAC63 | CPC | O-cresolphthalein complexone | 488 | 660 | Chemical Book | 2411-89-4 |
| ISAC65 | Cy2 | | 492 | 507 | GElifesciences | 102185-03-5 |
| ISAC66 | Cy3 | | 552 | 566 | GElifesciences | 146368-16-3 |
| ISAC67 | Cy3.5 | | 581 | 598 | GElifesciences | 189767-45-1 |
| ISAC68 | Cy5 | | 633 | 670 | GElifesciences | 144377-05-9 |
| ISAC69 | Cy5.5 | | 677 | 695 | GElifesciences | 210892-23-2 |
| ISAC70 | Cy7 | | 743 | 767 | GElifesciences | 169799-14-8 |
| ISAC71 | Cychrome | | 565 | 667 | BDBioscences | 245670-67-1 |
| ISAC73 | CyQUANT DNA | | 502 | 522 | LifeTechnologies | No names |
| | | | | | | found |
| ISAC74 | CyTRAK Orange | 1,5-bis{[2-(di-methylamino) | 514 | 609 | Abcam | 1195771-25-5 |
| | | ethyl]amino)-4,8- | | | (eBioscience) | |
| | | dihydroxyanthracene-9,10-dione | | | | |
| ISAC76 | DAPI | | 358 | 462 | PubChem | 47165-04-8 |
| ISAC77 | DCFH | | 505 | 525 | SigmaAldrich | 106070-31-9 |
| ISAC79 | DiA | DiA; 4-Di-16-ASP (4-(4- | 455 | 586 | LifeTechnologies | 371114-38-4 |
| | | (Dihexadecylamino)styryl)-N- | | | | |
| | | Methylpyridinium Iodide); | | | | |
| | | C46H79IN2 | | | | |
| ISAC81 | DiD | DiD′ solid; DiIC18(5) solid (1,1′- | 647 | 669 | LifeTechnologies | 127274-91-3 |
| | | Dioctadecyl-3,3,3′,3′- | | | | |
| | | Tetramethylindodicarbocyanine, | | | | |
| | | 4-Chlorobenzenesulfonate Salt); | | | | |
| | | C67H103ClN2O3S | | | | |
| ISAC84 | DiI | DiI Stain (1,1′-Dioctadecyl- | 550 | 568 | LifeTechnologies | 41085-99-8 |
| | | 3,3,3′,3′- | | | | |
| | | Tetramethylindocarbocyanine | | | | |
| | | Perchlorate (‘DiI’; DiIC18(3))); | | | | |
| | | C59H97ClN2O4; 3H-Indolium, 2- | | | | |
| | | (3-(1,3-dihydro-3,3-dimethyl-1- | | | | |
| | | octadecyl-2H-indol-2-ylidene)-1- | | | | |
| | | propenyl)-3,3-dimethyl-1- | | | | |
| | | octadecyl-, perchlorate/ | | | | |
| ISAC88 | DiO | DiO′; DiOC18(3) (3,3′- | 489 | 506 | LifeTechnologies | 34215-57-1 |
| | | Diociadecyloxacarbocyanine | | | | |
| | | Perchlorate); C53H85ClN2O6; | | | | |
| | | Benzoxazolium, 3-octadecyl-2- | | | | |
| | | [3-(3-octadecyl-2(3H)- | | | | |
| | | benzoxazolylidene)-1-propenyl]-, | | | | |
| | | perchlorate/ | | | | |
| ISAC92 | DiR | DiR′; DiIC18(7) (1,1′- | 750 | 781 | LifeTechnologies | 100068-60-8 |
| | | Dioctadecyl-3,3,3′,3′- | | | | |
| | | Tetramethylindotricarbocyanine | | | | |
| | | Iodide); C63H101IN2 | | | | |
| ISAC95 | DRAQ5 | | 645 | 683 | CellSignalingTech | 254098-36-7 |
| ISAC96 | DRAQ7 | | 599 | 694 | CellSignalingTech | 1533453-55-2 |
| ISAC97 | DsRED | | 532 | 595 | Clontech | 469863-23-8 |
| ISAC98 | dsRed2-RFP | | 555 | 582 | Clontech | No names |
| | | | | | | found |
| ISAC99 | DY547 | 547 Dyomics | 557 | 574 | Dynomics | 947138-67-2 |
| ISAC100 | DY634 | 634 Dyomics | 635 | 658 | Dynomics | 1189010-49-8 |
| ISAC101 | DY647 | 647 Dyomics | 650 | 665 | Dynomics | 890317-39-2 |
| ISAC102 | DyLight 350 | DL350 | 353 | 432 | PierceNet | 1436849-83-0 |
| ISAC103 | DyLight 405 | DL405 | 400 | 420 | PierceNet | 1051927-09-3 |
| ISAC104 | DyLight 488 | DL488 | 493 | 518 | PierceNet | 1051927-12-8 |
| ISAC105 | DyLight 549 | DL549 | 562 | 576 | JacksonImmunoRes | 1051927-13-9 |
| ISAC106 | DyLight 550 | DL550 | 562 | 576 | PierceNet | 1340586-78-8 |
| ISAC107 | DyLight 594 | DL594 | 593 | 618 | PierceNet | 1268612-00-5 |
| ISAC108 | DyLight 633 | DL633 | 638 | 658 | PierceNet | 1051927-14-0 |
| ISAC109 | DyLight 649 | DL649 | 654 | 670 | JacksonImmunoRes | 1051927-15-1 |
| ISAC110 | DyLight 650 | DL650 | 652 | 672 | PierceNet | 1364214-13-0 |
| ISAC111 | DyLight 680 | DL680 | 682 | 712 | PierceNet | 1051927-24-2 |
| ISAC112 | DyLight 800 | DL800 | 777 | 794 | PierceNet | 1051927-23-1 |
| ISAC113 | EB | Ethidium Bromide | 523 | 604 | SigmaAldrich | 1239-45-8 |
| ISAC114 | ECD | | 563 | 613 | LifeTechnologies | 88475-75-6 |
| ISAC116 | ECFP | enhanced cyan fluorescent | 435 | 477 | MyBiosource | No names |
| | | protein | | | | found |
| ISAC118 | EdU | EdU(5-ethynyl-2\u2032- | 0 | 0 | LifeTechnologies | 61135-33-9 |
| | | deoxyuridine); C11H12N2O5 | | | | |
| ISAC120 | EdU Alexa Fluor 488 | | 496 | 516 | LifeTechnologies | No names |
| | | | | | | found |
| ISAC121 | EdU Alexa Fluor 647 | | 650 | 665 | LifeTechnologies | No names |
| | | | | | | found |
| ISAC122 | EdU Pacific Blue | | 405 | 455 | LifeTechnologies | No names |
| | | | | | | found |
| ISAC123 | eFluor 450 | | 400 | 450 | eBioscience | 1592653-87-6 |
| ISAC124 | eFluor 450 Fixable | | 400 | 450 | eBioscience | No names |
| | Viability Dye | | | | | found |
| ISAC125 | eFluor 490 | | 350 | 490 | eBioscience | No names |
| | | | | | | found |
| ISAC126 | eFluor 506 Fixable | | 420 | 506 | eBioscience | No names |
| | Viability Dye | | | | | found |
| ISAC127 | eFluor 525 | | 350 | 525 | eBioscience | No names |
| | | | | | | found |
| ISAC128 | eFluor 565 | | 350 | 565 | eBioscience | No names |
| | | | | | | found |
| ISAC129 | eFluor 585 | | 350 | 604 | eBioscience | No names |
| | | | | | | found |
| ISAC130 | eFluor 605 | | 350 | 605 | eBioscience | 1248429-27-7 |
| ISAC131 | eFluor 615 | | 590 | 622 | eBioscience | No names |
| | | | | | | found |
| ISAC132 | eFluor 625 | | 350 | 625 | eBioscience | No names |
| | | | | | | found |
| ISAC133 | eFluor 650 | | 350 | 650 | eBioscience | No names |
| | | | | | | found |
| ISAC134 | eFluor 660 | | 633 | 658 | eBioscience | 1634649-16-3 |
| ISAC135 | eFluor 670 | | 0 | 0 | eBioscience | 1437243-07-6 |
| ISAC136 | eFluor 700 | | 350 | 700 | eBioscience | No names |
| | | | | | | found |
| ISAC137 | eFluor 710 | | 350 | 710 | eBioscience | No names |
| | | | | | | found |
| ISAC138 | eFluor 780 Fixable | | 755 | 780 | eBloscience | No names |
| | Viability Dye | | | | | found |
| ISAC139 | EGFP | enhanced green fluorescent | 480 | 510 | MyBiosource | No names |
| | | protein | | | | found |
| ISAC141 | Emerald 300 | | 289 | 530 | LifeTechnologies | No names |
| | | | | | | found |
| ISAC142 | Eosin | | 525 | 546 | SigmaAldrich | 17372-87-1 |
| ISAC143 | Ethidium Homodimer-1 | | 528 | 617 | SigmaAldrich | 61926-22-5 |
| ISAC144 | Ethidium Monoazide | | 510 | 590 | SigmaAldrich | 58880-05-0 |
| | EMA | | | | | |
| ISAC145 | EYFP | enhanced yellow fluorescent | 515 | 528 | MyBiosource | No names |
| | | protein | | | | found |
| ISAC147 | FAM | | 492 | 518 | PubChem | 76823-03-5 |
| ISAC149 | FITC | Fluorescein | 500 | 520 | PubChem | 27072-45-3 |
| ISAC153 | Fluo-3 | C51H50Cl2N2O23; Glycine, N- | 506 | 526 | LifeTechnologies | 123632-39-3 |
| | | [4-[6-[(acetyloxy)methoxy]-2,7- | | | | |
| | | dichloro-3-oxo-3H-xanthen-9-yl]- | | | | |
| | | 2-[2-[2-[bis[2- | | | | |
| | | [(acetyloxy)methoxy]-2- | | | | |
| | | oxyethyl]amino]-5- | | | | |
| | | methylphenoxy]ethoxy]phenyl]- | | | | |
| | | N-[2-[(acetyloxy)methoxy]-2- | | | | |
| | | oxyethyl]-, (acetyloxy)methyl | | | | |
| | | ester/ | | | | |
| ISAC155 | Fluo-4 | C51H50F2N2O23; Glycine, N- | 494 | 516 | LifeTechnologies | 273221-59-3 |
| | | [4-[6-[(acetyloxy)methoxy]-2,7- | | | | |
| | | difluoro-3-oxo-3H-xanthen-9-yl]- | | | | |
| | | 2-[2-[2-[bis[2- | | | | |
| | | [(acetyloxy)methoxy]-2- | | | | |
| | | oxoethyl]amino]-5- | | | | |
| | | methylphenoxy]ethoxy]phenyl]- | | | | |
| | | N-[2-[(acetyloxy)methoxy]-2- | | | | |
| | | oxoethyl]-, (acetyloxy)methyl | | | | |
| | | ester/ | | | | |
| ISAC152 | FLMA | Fluorescein-5- maleimide | 495 | 520 | PierceNet | 75350-46-8 |
| ISAC157 | Fluoro-Emerald | Dextran, Fluorescein, 10,000 | 495 | 523 | LifeTechnologies | 194369-11-4 |
| | | MW, Anionic, Lysine | | | | |
| | | Fixable | | | | |
| ISAC159 | Fura Red | | | | LifeTechnologies | 149732-62-7 |
| ISAC162 | Fura3 | Fura-2 LeakRes (AM) | 325 | 510 | SigmaAldrich | 172890-84-5 |
| ISAC164 | FxCycle Far Red | | 640 | 658 | LifeTechnologies | No names |
| | | | | | | found |
| ISAC165 | FxCycle Violet | C16H17Cl2N5; 1H-Indole-6- | 358 | 462 | LifeTechnologies | 28718-90-3 |
| | | carboximidamide, 2-[4- | | | | |
| | | (aminoiminomethyl)phenyl]-, | | | | |
| | | dihydrochloride/ | | | | |
| ISAC167 | GFP | green fluorescent protein | 488 | 515 | MyBiosource | No names |
| | | | | | | found |
| ISAC169 | GFP Violet Excited | | 398 | 515 | MyBiosource | No names |
| | | | | | | found |
| ISAC170 | GFP-Vex1 | | 398 | 515 | MyBiosource | No names |
| | | | | | | found |
| ISAC171 | HiLyte Fluor 488 | | 501 | 527 | Anaspec | 1051927-29-7 |
| ISAC172 | HiLyte Fluor 555 | | 550 | 566 | Anaspec | 1051927-30-0 |
| ISAC173 | HiLyte Fluor 647 | | 649 | 674 | Anaspec | 925693-87-4 |
| ISAC174 | HiLyte Fluor 680 | | 0 | 0 | Anaspec | 1051927-34-4 |
| ISAC175 | HiLyte Fluor 750 | | 754 | 778 | Anaspec | 1051927-32-2 |
| ISAC176 | Hoechst 33258 | | 345 | 455 | SigmaAldrich | 23491-45-4 |
| ISAC177 | Hoechst 33342 | bisBenzimide H 33342 | 343 | 455 | SigmaAldrich | 23491-52-3 |
| | | trihydrochloride | | | | |
| ISAC179 | Hydroxycoumarin | C10H6O5; 7-hydroxycoumarin- | 360 | 450 | LifeTechnologies | 43070-85-5 |
| | | 3-carboxylic acid; 2H-1- | | | | |
| | | Benzopyran-3-carboxylic acid, 7- | | | | |
| | | hydroxy-2-oxo-/; 4-chloromethyl- | | | | |
| | | 7-hydroxycoumarin | | | | |
| ISAC183 | Indo-1 | Indo-1 AM Calcium Sensor Dye; | 347 | 480 | LifeTechnologies | 96314-96-4 |
| | | C47H51N3O22; 1H-Indole-6- | | | | |
| | | carboxylic acid, 2-[4-[bis[2- | | | | |
| | | [(acetyloxy)methoxy]-2- | | | | |
| | | oxoethyl]amino]-3-[2-[2-[bis[2- | | | | |
| | | [(acetyloxy)methoxy]-2- | | | | |
| | | oxoetyl]amino]-5- | | | | |
| | | methylphenoxy]ethoxy]phenyl]-, | | | | |
| | | (acetyloxy)methyl ester/ | | | | |
| ISAC187 | JC-1 | 5,5′,6,6′-tetrachloro-1,1′,3,3′- | 593 | 595 | LifeTechnologies | 3520-43-2 |
| | | tetraethylbenzimidazolylcarbocyanine | | | | |
| | | iodide; C25H27Cl4IN4 | | | | |
| ISAC189 | Krome Orange | | 398 | 530 | Beckman Coulter | 1558035-65-6 |
| ISAC190 | Leadmium | | 490 | 520 | LifeTechnologies | No names |
| | | | | | | found |
| ISAC191 | LIVE/DEAD Fixable | Aqua LIVE/DEAD | 367 | 526 | LifeTechnologies | No names |
| | Aqua Dead Cell Stain | | | | | found |
| ISAC193 | LIVE/DEAD Fixable | Blue LIVE/DEAD | 343 | 442 | LifeTechnologies | No names |
| | Blue Dead Cell Stain | | | | | found |
| ISAC195 | LIVE/DEAD Fixable | | 650 | 670 | LifeTechnologies | No names |
| | Far Red Dead Cell | | | | | found |
| | Stain | | | | | |
| ISAC196 | LIVE/DEAD Fixable | Green LIVE/DEAD | 498 | 525 | LifeTechnologies | No names |
| | Green Dead Cell Stain | | | | | found |
| ISAC198 | LIVE/DEAD Fixable | | 752 | 776 | LifeTechnologies | No names |
| | Near-IR Dead Cell | | | | | found |
| | Stain | | | | | |
| ISAC199 | LIVE/DEAD Fixable | | 594 | 612 | LifeTechnologies | No names |
| | Red Dead Cell Stain | | | | | found |
| ISAC200 | LIVE/DEAD Fixable | Violet LIVE/DEAD | 403 | 455 | LifeTechnologies | No names |
| | Violet Dead Cell Stain | | | | | found |
| ISAC202 | LIVE/DEAD Fixable | Yellow LIVE/DEAD | 401 | 551 | LifeTechnologies | No names |
| | Yellow Dead Cell Stain | | | | | found |
| ISAC204 | Lucifer Yellow | C13H9Li2N5O9S2; 1H- | 428 | 544 | LifeTechnologies | 82446-52-4 |
| | | Benz[de]isoquinoline-5,8- | | | | |
| | | disulfonic acid, 6-amino-2- | | | | |
| | | [(hydrazinocarbonyl)amino]-2,3- | | | | |
| | | dihydro-1,3-dioxo-, dilithium salt/ | | | | |
| ISAC206 | Magnesium Green | C33H17Cl2K5N2O13; Glycine, | 507 | 531 | LifeTechnologies | 170516-41-3 |
| | | N-[2-(carboxymethoxy)-4-[[(2′,7′- | | | | |
| | | dichloro-3′,6′-dihydroxy-3- | | | | |
| | | oxospiro[isobenzofuran-1(3H),9′- | | | | |
| | | [9H]xanthen]-5- | | | | |
| | | yl)carbonyl]amino]phenyl]-N- | | | | |
| | | (carboxymethyl)-, | | | | |
| | | pentapotassium salt/ | | | | |
| ISAC208 | Marina Blue | C16H11F2NO7; 2,5- | 364 | 461 | LifeTechnologies | 215868-23-8 |
| | | Pyrrolidinedione, 1-[[(6,8- | | | | |
| | | difluoro-7-hydroxy-4-methyl-2- | | | | |
| | | oxo-2H-1-benzopyrar-3- | | | | |
| | | yl)acetyl]oxy)-/; | | | | |
| ISAC210 | mBanana | | 540 | 553 | Clontech | 1114839-40-5 |
| ISAC211 | mCherry | | 587 | 610 | Clontech | 1628764-31-7 |
| ISAC212 | mCitrine | | 516 | 529 | Not | 1357606-54-2 |
| | | | | | Commercialized | |
| ISAC213 | MethylCoumarin | AMCA-X, SE (6-((7-Amino-4- | 360 | 448 | LifeTechnologies | 1333-47-7 |
| | | Methylcoumarin-3- | | | | |
| | | Acetyl)amino)Hexanoic Acid, | | | | |
| | | Succinimidyl Ester); | | | | |
| | | C22H25N3O7 | | | | |
| ISAC216 | MitoTracker Green | C34H28Cl5N3O; | 490 | 512 | LifeTechnologies | 1304563-13-0 |
| | | Benzoxazolium, 2-[3-[5,6- | | | | |
| | | dichloro-1,3-bis[[4- | | | | |
| | | (chloromethyl)phenyl]methyl]- | | | | |
| | | 1,3-dihydro-2H-benzimidazol-2- | | | | |
| | | ylidene]-1-propenyl]-3-methyl-, | | | | |
| | | chloride/ | | | | |
| ISAC218 | MitoTracker Orange | C24H24Cl2N2O | 550 | 575 | LifeTechnologies | No names |
| | | | | | | found |
| ISAC219 | MitoTracker Red | C39H36Cl5N3 | 578 | 598 | LifeTechnologies | No names |
| | | | | | | found |
| ISAC220 | mOrange | | 548 | 562 | Clontech | 1114839-60-9 |
| ISAC221 | mPlum | | 590 | 649 | Clontech | 1399820-93-9 |
| ISAC222 | mRaspberry | | 597 | 624 | Clontech | 1452799-41-5 |
| ISAC223 | mRFP1 | | 584 | 607 | Not | 1452799-30-2 |
| | | | | | Commercialized | |
| ISAC224 | mStrawberry | | 574 | 596 | Clontech | 1114834-99-9 |
| ISAC225 | Na-Green | Sodium Green ™, | 506 | 532 | LifeTechnologies | 195244-55-4 |
| | | tetra(tetramethylammonium) | | | | |
| | | salt; C84H100Cl4N8O19 | | | | |
| ISAC228 | Nile Red | C20H18N2O2; 5H- | 559 | 637 | LifeTechnologies | 7385-67-3 |
| | | Benzo[\u03B1]phenoxazin-5- | | | | |
| | | one, 9-(diethylamino)-/ | | | | |
| ISAC230 | Oregon Green | | 491 | 519 | LifeTechnologies | 195136-58-4 |
| ISAC232 | Oregon Green 488-X, | | 500 | 525 | LifeTechnologies | 890416-18-9 |
| | succinimidyl ester | | | | | |
| ISAC233 | Oregon Green 514 | Oregon Green ® 514 carboxylic | 510 | 532 | LifeTechnologies | 198139-53-6 |
| | | acid, succinimidyl ester; | | | | |
| | | C26H12F5NO9S | | | | |
| ISAC235 | Pacific Blue | PacBlue; Pacific | 405 | 455 | LifeTechnologies | 215868-31-8 |
| | | Blue ™succinimidyl ester; | | | | |
| | | C14H7F2NO7 | | | | |
| ISAC236 | Pacific Blue | | 405 | 455 | LifeTechnologies | 215868-33-0 |
| | succinimidyl ester | | | | | |
| ISAC237 | Pacific Orange | PacOrange | 403 | 551 | LifeTechnologies | 1122414-42-9 |
| ISAC240 | PE-Alexa Fluor 610 | RPE-AF610 | 563 | 628 | LifeTechnologies | No names |
| | | | | | | found |
| ISAC241 | PE-Alexa Fluor 647 | RPE-AF647 | 567 | 669 | LifeTechnologies | No names |
| | | | | | | found |
| ISAC242 | PE-Alexa Fluor 680 | RPE-AF680 | 570 | 702 | LifeTechnologies | No names |
| | | | | | | found |
| ISAC243 | PE-Alexa Fluor 700 | RPE-AF700 | 563 | 720 | LifeTechnologies | No names |
| | | | | | | found |
| ISAC244 | PE-Alexa Fluor 750 | RPE-AF750 | 570 | 776 | AbD Serotec | No names |
| | | | | | | found |
| ISAC245 | PE-CF594 | PE-Dazzle 594 | 564 | 612 | BDBioscences | 1613592-67-8 |
| ISAC72 | PE-Cy5 | | 565 | 667 | BDBioscences | 1448849-77-1 |
| ISAC248 | PE-Cy5.5 | | 563 | 695 | AbD Serotec | No names |
| | | | | | | found |
| ISAC249 | PE-Cy7 | | 563 | 760 | AbD Serotec | 1429496-42-3 |
| ISAC250 | PE-DY590 | | 563 | 599 | LSBio | No names |
| | | | | | | found |
| ISAC251 | PE-DY647 | | 563 | 672 | LSBio | No names |
| | | | | | | found |
| ISAC252 | PerCP | | 490 | 675 | AbD Serotec | 422551-33-5 |
| ISAC253 | PerCP-Cy5.5 | | 488 | 695 | AbD Serotec | 1474026-81-7 |
| ISAC254 | PerCP-eFluor 710 | | 488 | 710 | eBioscience | 1353683-31-4 |
| ISAC115 | PE-Texas Red | | 563 | 613 | LifeTechnologies | No names |
| | | | | | | found |
| ISAC256 | PE-Vio770 | | 565 | 775 | Miltenyl Biotech | No names |
| | | | | | | found |
| ISAC257 | pHrodo | pHrodo ™ Red, succinimidyl | 560 | 586 | LifeTechnologies | No names |
| | | ester (pHrodo ™ Red, SE); | | | | |
| | | pHrodo ™ Green STP Ester | | | | |
| ISAC260 | pHrodo Green STP | | 560 | 586 | LifeTechnologies | No names |
| | Ester | | | | | found |
| ISAC258 | pHrodo Red, | | 560 | 586 | LifeTechnologies | No names |
| | succinimidyl ester | | | | | found |
| ISAC261 | Phycocyanin | | 617 | 646 | SigmaAldrich | 11016-15-2 |
| ISAC262 | PicoGreen | Quant-iT ™ PicoGreen ® dsDNA | 502 | 522 | LifeTechnologies | 177571-06-1 |
| | | Reagent | | | | |
| ISAC264 | PKH2 | PKH2 Green Fluorescent Cell | 490 | 504 | SigmaAldrich | 145687-07-6 |
| | | Linker | | | | |
| ISAC266 | PKH26 | PKH26 Red Fluorescent Cell | 551 | 567 | SigmaAldrich | 154214-55-8 |
| | | Linker | | | | |
| ISAC268 | PKH67 | PKH67 Green Fluorescent Cell | 490 | 504 | SigmaAldrich | 257277-27-3 |
| | | Linker | | | | |
| ISAC270 | POPO-1 | C41H54I4N6O2; | 433 | 457 | LifeTechnologies | 169454-15-3 |
| | | Benzoxazolium, 2,2′-[1,3- | | | | |
| | | propanediylbis[(dimethyliminio)- | | | | |
| | | 3,1-propanediyl-1(4H)-pyridinyl- | | | | |
| | | 4-ylidenemethylidyne]]bis[3- | | | | |
| | | methyl]-, tetraiodide/ | | | | |
| ISAC272 | PO-PRO-1 | C20H27I2N3O; Benzoxazolium, | 435 | 457 | LifeTechnologies | 157199-56-9 |
| | | 3-methyl-2-[[1-[3- | | | | |
| | | (trimethylammonio)propyl]- | | | | |
| | | 4(1H)-pyridinylidene]methyl]-; | | | | |
| | | diiodide/; | | | | |
| ISAC274 | Propidium Iodide | C27H34I2N4 Phenanthridinium, | 350 | 617 | LifeTechnologies | 25535-16-4 |
| | | 3,8-diamino-5-[3- | | | | |
| | | (diethylmethylammonio)propyl]- | | | | |
| | | 6-phenyl-, diiodide | | | | |
| ISAC276 | PURE | | 0 | 0 | Not | No names |
| | | | | | Commercialized | found |
| ISAC277 | Pyronin Y | | 547 | 560 | SigmaAldrich | 92-32-0 |
| ISAC278 | Qdot 525 | | 350 | 525 | LifeTechnologies | 885332-45-6 |
| ISAC279 | Qdot 545 | | 350 | 545 | LifeTechnologies | 948906-89-6 |
| ISAC280 | Qdot 565 | | 350 | 565 | LifeTechnologies | 859509-02-7 |
| ISAC281 | Qdot 585 | | 350 | 585 | LifeTechnologies | 885332-46-7 |
| ISAC282 | Qdot 605 | | 350 | 605 | LifeTechnologies | 849813-89-4 |
| ISAC283 | Odot 625 | | 350 | 625 | LifeTechnologies | 1144512-19-5 |
| ISAC284 | Qdot 655 | | 350 | 655 | LifeTechnologies | 674287-64-0 |
| ISAC285 | Qdot 705 | | 350 | 705 | LifeTechnologies | 885332-47-8 |
| ISAC286 | Qdot 800 | | 350 | 800 | LifeTechnologies | 885332-50-3 |
| ISAC287 | RD1 | R-Phycoerythrin | 563 | 578 | LifeTechnologies | 1376573-14-6 |
| ISAC295 | Rhodamine | | 550 | 570 | LifeTechnologies | No names |
| | | | | | | found |
| ISAC290 | Rho 110 | Rhodamine 110 | 497 | 520 | LifeTechnologies | 13558-31-1 |
| ISAC293 | Rho 123 | Rhodamine 123 | 507 | 529 | LifeTechnologies | 62669-70-9 |
| ISAC296 | Rhodamine Green | Rhodamine Green ™carboxylic | 505 | 527 | LifeTechnologies | 189200-71-3 |
| | | acid, succinimidyl ester, | | | | |
| | | hydrochloride; C25H18ClN3O7 | | | | |
| ISAC297 | Rhodamine Green carboxylic acid, succinimidyl ester, | | 505 | 527 | LifeTechnologies | 254732-34-8 |
| | hydrochloride | | | | | |
| ISAC298 | Rhodamine Red | | 573 | 591 | LifeTechnologies | 99752-92-8 |
| ISAC299 | Rhodamine Red-X | Rhodamine Red ™-X, succinimidyl | 570 | 576 | LifeTechnologies | 178623-12-6 |
| | | ester; C37H44N4O10S2 | | | | |
| ISAC300 | Rhodamine Red-X, | | 570 | 576 | LifeTechnologies | 178623-13-7 |
| | succinimidyl ester | | | | | |
| ISAC301 | RiboFlavin | | 266 | 531 | SigmaAldrich | 83-88-5 |
| ISAC239 | R-Phycoerythrin | PE | 563 | 578 | LifeTechnologies | 11016-17-4 |
| ISAC303 | SNARF-1 carboxylic acid, acetate, succinimidyl ester | | 549 | 586 | LifeTechnologies | No names |
| | | | | | | found |
| ISAC302 | SNARF-1 pH 6 | SNARF ®-1 carboxylic acid, | 549 | 586 | LifeTechnologies | No names |
| | | acetate, succinimidyl ester; | | | | found |
| | | C33H24N2O9 | | | | |
| ISAC304 | SNARF-1 pH 9 | | 576 | 640 | LifeTechnologies | No names |
| | | | | | | found |
| ISAC305 | Spectral Red | | 506 | 665 | MyBiosource | No names |
| | | | | | | found |
| ISAC306 | SureLight P1 | | 545 | 667 | Abcam (Columbia | No names |
| | | | | | Biosciences) | found |
| ISAC307 | SureLight P3 | | 614 | 662 | Abcam | 1365659-06-8 |
| ISAC308 | SureLight PBXL-3 | | 614 | 662 | Abcam | No names |
| | | | | | | found |
| ISAC309 | SYBR Green | | 498 | 522 | SigmaAldrich | 217087-73-5 |
| ISAC310 | SYTO 11 | | 506 | 526 | LifeTechnologies | 173080-67-6 |
| ISAC311 | SYTO 13 | | 488 | 506 | LifeTechnologies | 173080-69-8 |
| ISAC312 | SYTO 16 | | 488 | 520 | LifeTechnologies | 173080-72-3 |
| ISAC313 | SYTO 17 | | 618 | 637 | LifeTechnologies | 189233-66-7 |
| ISAC314 | SYTO 45 | | 450 | 486 | LifeTechnologies | 335078-86-9 |
| ISAC315 | SYTO 59 | | 622 | 643 | LifeTechnologies | 235422-34-1 |
| ISAC316 | SYTO 60 | | 650 | 681 | LifeTechnologies | 335079-14-6 |
| ISAC317 | SYTO 61 | | 618 | 651 | LifeTechnologies | 335079-15-7 |
| ISAC318 | SYTO 62 | | 650 | 681 | LifeTechnologies | 286951-08-4 |
| ISAC319 | SYTO 82 | | 540 | 560 | LifeTechnologies | 335079-10-2 |
| ISAC320 | SYTO 9 | | 482 | 500 | LifeTechnologies | 208540-89-0 |
| ISAC321 | SYTOX AADvanced | | 546 | 646 | LifeTechnologies | No names |
| | | | | | | found |
| ISAC322 | SYTOX Blue | | 431 | 480 | LifeTechnologies | 396077-00-2 |
| ISAC323 | SYTOX Green | | 504 | 523 | LifeTechnologies | 194100-76-0 |
| ISAC324 | SYTOX Orange | | 547 | 570 | LifeTechnologies | 324767-53-5 |
| ISAC325 | SYTOX Red | | 640 | 658 | LifeTechnologies | 915152-67-9 |
| ISAC326 | tdTomato | | 554 | 581 | Clontech | 1114838-94-6 |
| ISAC334 | Tetramethylrhodamine | TMRho | 553 | 581 | LifeTechnologies | 70281-37-7 |
| ISAC329 | Texas Red | Texas Red ®-X, succinimidyl ester; | 589 | 615 | LifeTechnologies | 82354-19-6 |
| | | C41H44N4O10S2 | | | | |
| ISAC330 | Texas Red-X, | | 589 | 615 | LifeTechnologies | 216972-99-5 |
| | succinimidyl ester | | | | | |
| ISAC331 | Thiazole Orange | | 500 | 530 | SigmaAldrich | 107091-89-4 |
| ISAC332 | ThiolTracker Violet | | 406 | 526 | LifeTechnologies | No names |
| | | | | | | found |
| ISAC335 | TO-PRO-1 | TO-PRO ®-1 iodide (515/531); | 509 | 533 | LifeTechnologies | 157199-59-2 |
| | | C24H29I2N3S; Quinolinium, 4-[(3- | | | | |
| | | methyl-2(3H)- | | | | |
| | | benzothiazolylidene)methyl]-1-[3- | | | | |
| | | (trimethylammonio)propyl]-, | | | | |
| | | diiodide/; | | | | |
| ISAC338 | TO-PRO-3 | TO-PRO ®-3 iodide (642/661); | 642 | 661 | LifeTechnologies | 157199-63-8 |
| | | C26H31I2N3S; Quinolinium, 4-[3- | | | | |
| | | (3-methyl-2(3H)- | | | | |
| | | benzothiazolylidene)-1-propenyl]-1- | | | | |
| | | [3-(trimethylammonio)propyl]-, | | | | |
| | | diiodide/ | | | | |
| ISAC341 | TOTO-1 | TOTO ®-1 iodide (514/533); | 509 | 533 | LifeTechnologies | 143413-84-7 |
| | | C49H58I4N6S2; Quinolinium, 1-1′- | | | | |
| | | [1,3- | | | | |
| | | propanediylbis[(dimethyliminio)- | | | | |
| | | 3,1-propanediyl]]bis[4-[(3-methyl- | | | | |
| | | 2(3H)- | | | | |
| | | benzothiazolylidene)methyl]]-, | | | | |
| | | tetraiodide/ | | | | |
| ISAC344 | TOTO-3 | TOTO ®-3 iodide (642/660); | 642 | 661 | LifeTechnologies | 166196-17-4 |
| | | C53H62I4N6S2 | | | | |
| ISAC346 | TriColor | | 563 | 670 | LifeTechnologies | 478184-50-8 |
| ISAC347 | TRITC | Tetramethylrhodamine; | 547 | 572 | LifeTechnologies | 745735-42-6 |
| | | tetramethylrhodamine-5-(and-6)- | | | | |
| | | isothiocyanate; C25H21N3O3S; | | | | |
| | | Xanthylium, 9-(2- | | | | |
| | | carboxyisothiocyanatophenyl)-3,6- | | | | |
| | | bis(dimethylamino)-, inner salt/ | | | | |
| ISAC351 | TruRed | | 490 | 695 | Not | 396076-95-2 |
| | | | | | Commercialized | |
| ISAC352 | V19 | | 397 | 572 | Not | No names |
| | | | | | Commercialized | found |
| ISAC353 | V450 | | 405 | 448 | BDBioscences | 1257844-82-8 |
| ISAC354 | V500 | | 415 | 500 | BDBioscences | 1333160-12-5 |
| ISAC355 | VioBlue | | 400 | 452 | Millenyl Biotech | 1431147-59-9 |
| ISAC356 | VioGreen | | 388 | 520 | Miltenyl Biotech | No names |
| | | | | | | found |
| ISAC357 | Vybrant DyeCycle | | 505 | 535 | LifeTechnologies | 1431152-50-9 |
| | Green | | | | | |
| ISAC358 | Vybrant DyeCycle | | 518 | 563 | LifeTechnologies | 1055990-89-0 |
| | Orange | | | | | |
| ISAC359 | Vybrant DyeCycle | | 637 | 686 | LifeTechnologies | 1345202-72-3 |
| | Ruby | | | | | |
| ISAC360 | Vybrant DyeCycle | | 370 | 436 | LifeTechnologies | 1015439-88-9 |
| | Violet | | | | | |
| ISAC361 | YFP | Yellow Fluorescent Protein | 505 | 530 | Clontech | No names |
| | | | | | | found |
| ISAC363 | YO-PRO-1 | YO-PRO ®-1 iodide (491/509); | 491 | 506 | LifeTechnologies | 152068-09-2 |
| | | C24H29I2N3O | | | | |
| ISAC365 | YO-PRO-3 | YO-PRO ®-3 iodide (612/631); | 613 | 629 | LifeTechnologies | 157199-62-7 |
| | | C26H31I2N3O; Quinolinium, 4-[3-(3- | | | | |
| | | methyl-2(3H)- | | | | |
| | | benzoxazolylidene)-1-propenyl]-1- | | | | |
| | | [3-(trimethylammonio)propyl]-, | | | | |
| | | diiodide/ | | | | |
| ISAC368 | YOYO-1 | YOYO ®-1 iodide (491/509); | 491 | 509 | LifeTechnologies | 143413-85-8 |
| | | C49H58I4N6O2; | | | | |
| ISAC370 | YOYO-3 | YOYO ®-3 iodide (612/631); | 613 | 629 | LifeTechnologies | 156312-20-8 |
| | | C53H62I4N6O2; Quinolinium, 1,1′ | | | | |
| | | [1,3- | | | | |
| | | propanediylbis[(dimethyliminio)- | | | | |
| | | 3,1-propanediyl]]bis[4-[3-(3-methyl- | | | | |
| | | 2(3H)-benzoxazolylidene)-1- | | | | |
| | | propenyl]]-, tetraiodide/; | | | | |
| ISAC373 | ZsGreen | | 494 | 517 | Clontech | 1216871-88-3 |

In embodiments, the hydrogel or polymer particle comprises a scatter-modulating additive. In embodiments, the scatter-modulating additive comprises polymer nanoparticles. In embodiments, the polymer nanoparticles comprise polystyrene. In embodiments, the scatter-modulating additive includes a co-monomer. In embodiments, the scatter-modulating additive includes a suspension of nanoparticles.

In embodiments, the hydrogel or polymer particle is a chemically functionalized hydrogel or polymer particle. In embodiments, the hydrogel comprises a free amine group. In embodiments, the hydrogel bead comprises allylamine. In embodiments, the hydrogel or polymer particle comprises biotin. In embodiments, the hydrogel or polymer particle comprises streptavidin. In embodiments, the hydrogel or polymer particle comprises avidin. In embodiments, the chemically functionalized hydrogel or polymer particle comprises an amine group, a carboxyl group, a hydroxyl group, or a combination thereof. In embodiments, the hydrogel or polymer particle comprises multiple bifunctional monomers to functionalize the hydrogel or polymer particle with different chemistries and/or molecules.

### Compositions

Compositions of the disclosure may comprise populations of polymer beads. In embodiments, the populations of polymer beads may comprise fluorophores, biomarkers, and/or the like. In embodiments, the populations of polymer beads may comprise up to 5, up to 10, up to 12, up to 18, up to 20, up to 30, up to 40, up to 50, up to 60, up to 70, up to 80, up to 90, or up to 100 populations of polymer beads. In embodiments, each bead population comprises a fluorophore, a biomarker, and/or the like.

In embodiments, each bead population may comprise a single fluorophore. In embodiments, each bead population may comprise a different fluorophore. In embodiments, each fluorophore emits fluorescence at one, two, three, four, five, six, seven, eight, or nine wavelengths. In embodiments, each fluorophore has a diameter between about 500 nm and about 10 μm.

For example, in an embodiment, a first population of polymer beads comprises a first fluorophore and a second population of polymer beads comprises a second fluorophore. The first fluorophore and the second fluorophore may each be selected from the fluorophores outlined above and may be the same or different. Moreover, a final population of polymer beads does not comprise any fluorophores.

In embodiments, each bead population may comprise a single biomarker. In embodiments, each bead population may comprise a different biomarker. For example, in an embodiment, a first population of polymer beads comprises a first biomarker and a second population of polymer beads comprises a second biomarker. The first biomarker and the second biomarker may each be selected from the biomarker outlined above and may be the same or different. Moreover, a final population of polymer beads does not comprise any biomarker.

### EXAMPLES

### Example 1—Deconvolution Using Separate Tubes

Representing a TBNK staining panel, six sets of antibody-fluorophore conjugates were prepared and attached directly to polymer particles. The following fluorophores were used: PerCP-Cy5.5, PE Cy7, APC-Cy7, FITC, PE, APC.

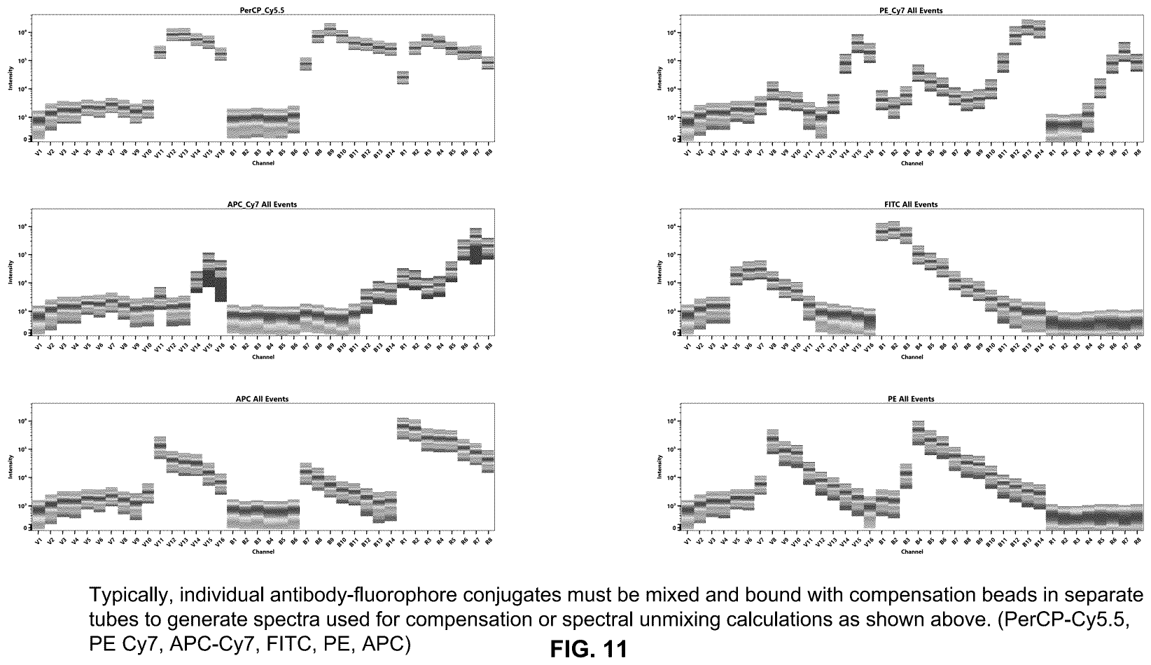

Six separate sample tubes were prepared with 100 μL of phosphate buffered saline (PBS). To each of such tubes, approximately 25,000 beads from each set were added. For the purposes of clarity, each sample tube contained only one set of antibody-fluorophore conjugates. A seventh sample tube was prepared as an unstained/negative control. Each sample tube was vortexed to resuspend the particles and analyzed on a Cytek Northern Lights™ flow cytometer using routine acquisition parameters. The unstained/negative control was then measured in the same manner. FIG. 11 illustrates the individual fluorophore signals for each sample tube.

### Example 2—One Pot Analysis and Compensation

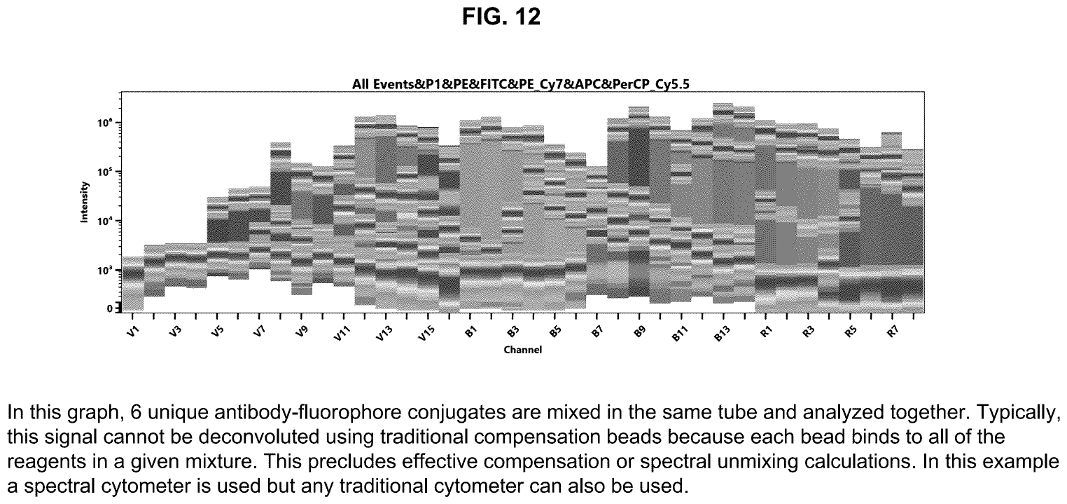

The six sets of polymer beads from Example 1 were combined into a single tube and analyzed on a Cytek Northern Lights™ using routine acquisition parameters, yielding a combined spectrum (FIG. 12). In this example, the individual bead-antibody-fluorophore particles uniquely contained only one type of fluorophore per bead.

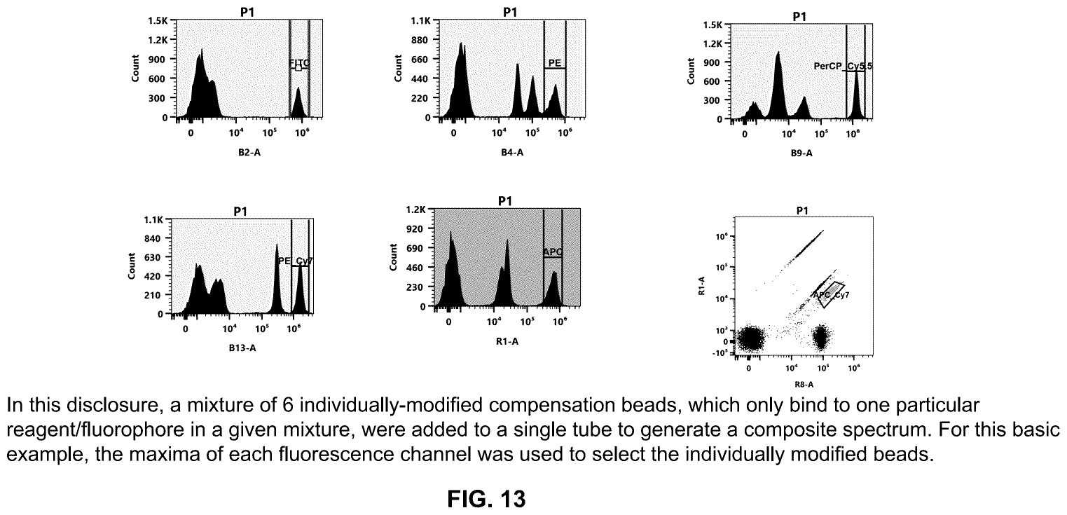

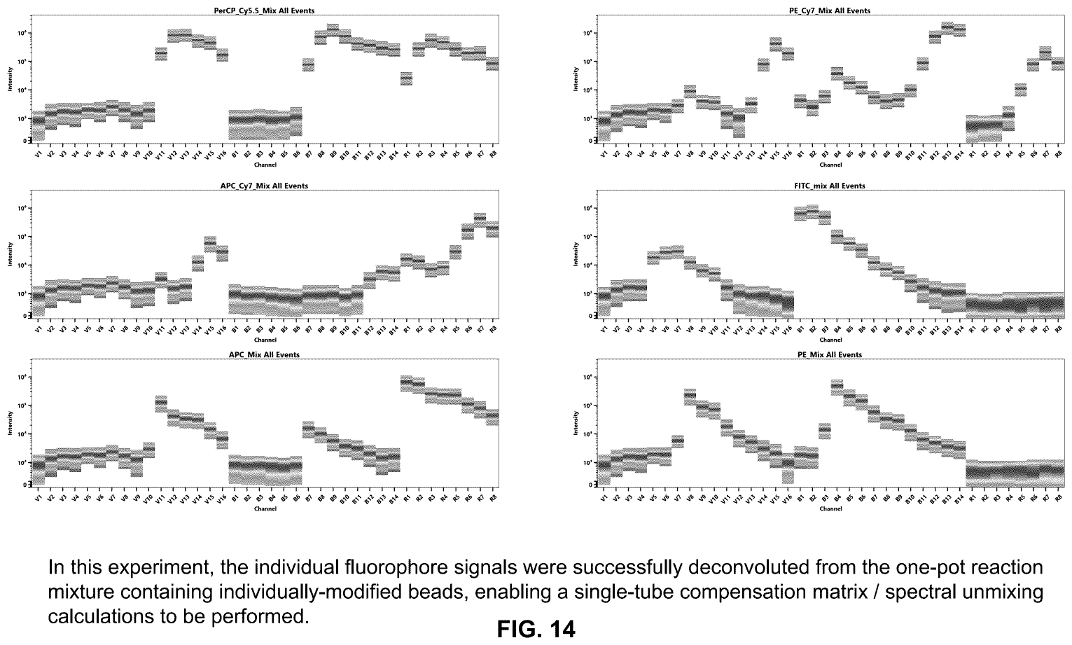

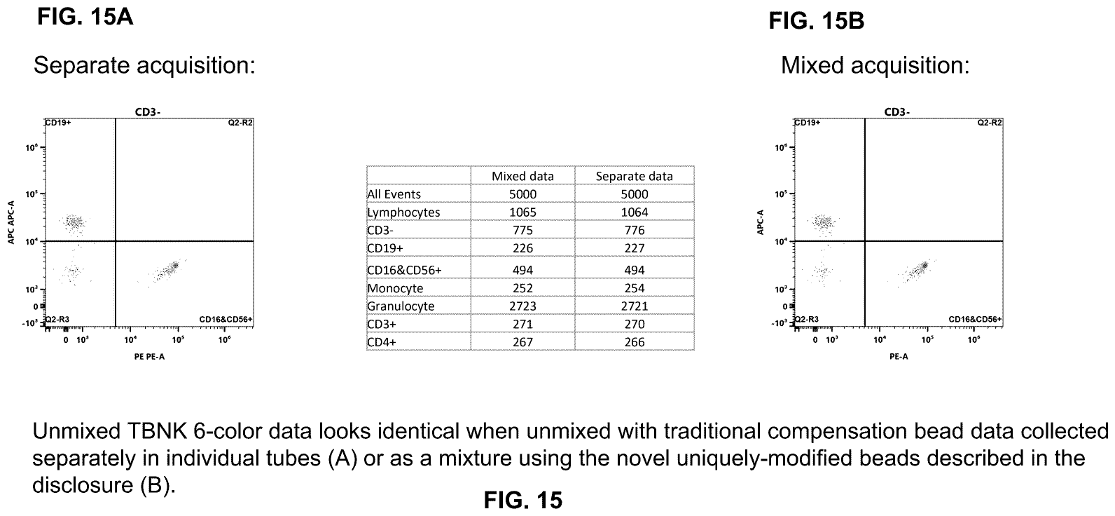

Using the known fluorescent signal of each antibody-fluorophore conjugate, the individual fluorophore signals from each polymer bound antibody-fluorophore were deconvoluted. Specifically, the fluorescence maxima associated with a given antibody-fluorophore conjugate from the staining antibody mixture was used to select the subset of beads that were bound to that specific antibody-fluorophore conjugate (FIG. 13). This allowed for the deconvolution of the full fluorescence signal for individual fluorophores, from the mixed reaction. The results of such deconvolution are shown in FIG. 14. The individual spectra seen in FIG. 14 were indistinguishable from those generated from separate tubes (FIG. 11) and could be used for downstream compensation or spectral unmixing calculations with equivalent performance to the individual, physically segregated tubes (FIG. 15). The resulting unmixed TBNK staining panel using the one pot polymer particle method described in this disclosure was indistinguishable from the staining panel using the separate tubes described in Example 1. See FIG. 15.

### Example 3—One Pot Compensation with Biomarker Modified Beads

Six sets of polymer beads, each containing a single biomarker from an example TBNK panel (CD3, CD16, CD56, CD45, CD4, CD19, CD8) are added to a single tube containing 100 μL of PBS. The full panel of antibody-fluorophore conjugates from a TBNK panel are next added to the same single reaction tube and vortexed to resuspend the mixture. After incubating the mixture under light protection, it is analyzed on a calibrated flow cytometer using routine acquisition parameters, yielding a combined spectrum. In this example, the individual biomarker-modified beads bind only to a specific antibody-fluorophore from the staining panel mixture, generating uniformly labeled sets of beads.

Using the known fluorescent signal of each antibody-fluorophore conjugate, the individual fluorophore signals from each polymer bound antibody-fluorophore are deconvoluted. Specifically, the fluorescence maxima associated with a given antibody-fluorophore conjugate from the staining antibody mixture is used to select the subset of beads that were bound to that specific antibody-fluorophore conjugate. This allows for the deconvolution of the full fluorescence signal for individual fluorophores, from the mixed reaction for use in compensation or spectral unmixing calculations.

### Example 4—One Pot Compensation with Pre-Modified Beads

The six sets of beads from Example 1 can be pre-modified with appropriate fluorophores, in contrast to antibody-fluorophore conjugates, to achieve the same effect.

### Example 5—Streamlined FMO Compensation with Single-Biomarker Beads

All possible combinations of the six sets of beads from Example 3, minus one biomarker-fluorophore channel, are added to individual tubes to represent an FMO staining control set. For the purposes of clarity, example sets include the following: (CD16, CD56, CD45, CD4, CD19, CD8), (CD3, CD56, CD45, CD4, CD19, CD8), (CD3, CD16, CD45, CD4, CD19, CD8), (CD3, CD16, CD56, CD4, CD19, CD8), (CD3, CD16, CD56, CD45, CD19, CD8), (CD3, CD16, CD56, CD45, CD4, CD8), and (CD3, CD16, CD56, CD45, CD4, CD19). In this product format, a single master mix of the TBNK staining panel can be added to all of the tubes to generate an FMO control matrix. Specifically, adding a single cocktail of anti CD3-FITC, anti CD16-PE, anti CD56-PE, anti CD45-PerCP Cy5.5, anti CD4 PE-Cy7, anti CD19-APC, and anti CD8-APC Cy7 to each of the pre-mixed FMO tubes described in the disclosure will allow a user to generate a full FMO panel. In each of these instances, the polymer particles in the tubes will bind to all but one of the antibody-fluorophore conjugates in the full TBNK panel, simulating a traditional FMO approach in a greatly streamlined product format. In contrast, using traditional methods would require the user to generate the combinatorial cocktail of antibodies in all combinations in order to achieve the same result, because all of the antibodies would bind to the cells or traditional compensation beads used for FMO calculations.

### Example 6—Streamlined FMO Compensation with Single-Biomarker Beads

The sets of beads in Example 5 can be pre-modified with appropriate fluorophore, or antibody-fluorophore conjugate combinations to achieve the same effect.

### Further Numbered Embodiments

Further embodiments of the instant invention are provided in the numbered embodiments below:

Embodiment 1. A composition comprising (i) a first population of polymer beads comprising a first fluorophore, and (ii) a second population of polymer beads comprising a second fluorophore.

Embodiment 2. The composition of Embodiment 1, comprising up to 5, up to 10, up to 12, up to 18, up to 20, up to 30, up to 40, up to 50, up to 60, up to 70, up to 80, up to 90, or up to 100 populations of polymer beads, wherein each bead population comprises a fluorophore, and wherein the fluorophore for each population of beads is different.

Embodiment 3. The composition of Embodiment 1, wherein the first fluorophore and the second fluorophore are different fluorophores.

Embodiment 3.1. The composition of any one of Embodiments 1-3, wherein each population of polymer beads comprises only a single type of fluorophore.

Embodiment 4. The composition of any one of Embodiments 1-3.1, further comprising a final population of polymer beads that do not comprise any fluorophores.

Embodiment 4.1. The composition of any one of Embodiments 1-4, wherein each fluorophore is independently selected from those listed in Table 4.

Embodiment 5. The composition of any one of Embodiments 1-4, wherein each fluorophore is independently selected from any one of: peridinin chlorophyll protein-cyanine 5.5 dye (PerCP-Cy5.5); phycoerythrin-cyanine7 (PE Cy7); allophycocyanin-cyanine 7 (APC-Cy7); fluorescein isothiocyanate (FITC); phycoerythrin (PE); allophyscocyanin (APC); 6-carboxy-4′,5′-dichloro-2′,7′-dimethoxyfluorescein succinimidylester; 5-(and-6)-carboxyeosin; 5-carboxyfluorescein; 6 carboxyfluorescein; 5-(and-6)-carboxyfluorescein; S-carboxyfluorescein-bis-(5-carboxymethoxy-2-nitrobenzyl)ether,-alanine-carboxamide, or succinimidyl ester; 5-carboxy fluorescein succinimidyl ester; 6-carboxyfluorescein succinimidyl ester; 5-(and-6)-carboxyfluorescein succinimidyl ester; 5-(4,6-dichlorotriazinyl)amino fluorescein; 2′,7-difluoro fluorescein; eosin-5-isothiocyanate; erythrosin5-isothiocyanate; 6-(fluorescein-5-carboxamido) hexanoic acid or succinimidyl ester; 6-(fluorescein-5-(and-6)-carboxamido) hexanoic acid or succinimidylester; fluorescein-S-EX succinimidyl ester; fluorescein-5-isothiocyanate; fluorescein-6-isothiocyanate; OregonGreen® 488 carboxylic acid, or succinimidyl ester; Oregon Green® 488 isothiocyanate; Oregon Green® 488-X succinimidyl ester; Oregon Green® 500 carboxylic acid; Oregon Green® 500 carboxylic acid, succinimidylester or triethylammonium salt; Oregon Green® 514 carboxylic acid; Oregon Green® 514 carboxylic acid or succinimidyl ester; RhodamineGreen™ carboxylic acid, succinimidyl ester or hydrochloride; Rhodamine Green™ carboxylic acid, trifluoroacetamide or succinimidylester; Rhodamine Green™-X succinimidyl ester or hydrochloride; RhodolGreen™ carboxylic acid, N,O-bis-(trifluoroacetyl) or succinimidylester; bis-(4-carboxypiperidinyl) sulfonerhodamine or di(succinimidylester); 5-(and-6)carboxynaphtho fluorescein, 5-(and-6)carboxynaphthofluorescein succinimidyl ester; 5-carboxyrhodamine 6G hydrochloride; 6-carboxyrhodamine6Ghydrochloride, 5-carboxyrhodamine 6G succinimidyl ester; 6-carboxyrhodamine 6G succinimidyl ester; 5-(and-6)-carboxyrhodamine6G succinimidyl ester; 5-carboxy-2′,4′,5′,7′-tetrabromosulfonefluorescein succinimidyl esteror bis-(diisopropylethylammonium) salt; 5-carboxytetramethylrhodamine; 6-carboxytetramethylrhodamine; 5-(and-6)-carboxytetramethylrhodamine; 5-carboxytetramethylrhodamine succinimidyl ester; 6-carboxytetramethylrhodaminesuccinimidyl ester; 5-(and-6)-carboxytetramethylrhodamine succinimidyl ester; 6-carboxy-X-rhodamine; 5-carboxy-X-rhodamine succinimidyl ester; 6-carboxy-X-rhodamine succinimidyl ester; 5-(and-6)-carboxy-X-rhodamine succinimidyl ester; 5-carboxy-X-rhodamine triethylammonium salt; Lissamine™ rhodamine B sulfonyl chloride; malachite green; isothiocyanate; NANOGOLD® mono(sulfosuccinimidyl ester); QSY® 21carboxylic acid or succinimidyl ester; QSY® 7 carboxylic acid or succinimidyl ester; Rhodamine Red™-X succinimidyl ester; 6-(tetramethylrhodamine-5-(and-6)-carboxamido) hexanoic acid; succinimidyl ester; tetramethylrhodamine-5-isothiocyanate; tetramethylrhodamine-6-isothiocyanate; tetramethylrhodamine-5-(and-6)-isothiocyanate; Texas Red® sulfonyl; Texas Red® sulfonyl chloride; Texas Red®-X STP ester or sodium salt; Texas Red®-X succinimidyl ester; Texas Red®-X succinimidyl ester; X-rhodamine-5-(and-6) isothiocyanate, BODIPY® FL; BODIPY® TMR STP ester; BODIPY® TR-X STP ester; BODIPY® 630/650-X STPester; BODIPY® 650/665-X STP ester; 6-dibromo-4,4-difluoro-5,7-dimethyl-4-bora-3a,4a-diaza-s-indacene-3-propionic acid succinimidyl ester; 4,4-difluoro-4-bora-3a,4a-diaza-s-indacene-3,5-dipropionic acid; 4,4-difluoro-5,7-dimethyl-4-bora-3a,4a-diaza-s-indacene-3-pentanoicacid; 4,4-difluoro-5,7-dimethyl-4-bora3a,4a-diaza-s-indacene-3-pentanoicacid succinimidyl ester; 4,4-difluoro-5,7-dimefhyl-4-bora-3a,4a-diaza-s-indacene-3propionicacid; 4,4-difluoro-5,7-dimethyl-4-bora-3a,4adiaza-s-indacene-3-propionicacid succinimidyl ester; 4,4-difluoro-5,7-dimefhyl-4-bora-3a,4a-diaza-s-indacene-3propionic acid; sulfosuccinimidyl ester or sodium salt; 6-((4,4-difluoro-5,7-dimethyl-4-bora-3a,4a-diaza-s-indacene-3propionyl)amino)hexanoicacid; 6-((4,4-difluoro-5,7 dimethyl-4-bora-3a,4a-diaza-s-indacene-3-propionyl)amino)hexanoic acid or succinimidyl ester; N-(4,4-difluoro 5,7-dimethyl-4-bora-3a,4a-diaza-s-indacene-3-propionyl) cysteic acid, succinimidyl ester or triethylammonium salt; 6-4,4-difluoro-1,3-dimethyl-5-(4-methoxyphenyl)-4-bora3a,4a4,4-difluoro-5,7-diphenyl-4-bora-3a,4a-diaza-sindacene-3-propionicacid; 4,4-difluoro-5,7-diphenyl-4-bora3a,4a-diaza-s-indacene-3-propionicacid succinimidyl ester; 4,4-difluoro-5-phenyl-4-bora-3a,4a-diaza-s-indacene-3-propionic acid; succinimidyl ester; 6-((4,4-difluoro-5-phenyl-4 bora-3a,4a-diaza-s-indacene-3-propionyl)amino) hexanoicacid or succinimidyl ester; 4,4-difluoro-5-(4-phenyl-1,3butadienyl)-4-bora-3a,4a-diaza-s-indacene-3-propionicacid succinimidyl ester; 4,4-difluoro-5-(2-pyrrolyl)-4-bora-3a,4a-diaza-s-indacene-3-propionic acid succinimidyl ester; 6-(((4,4-difluoro-5-(2-pyrrolyl)-4-bora-3a,4a-diaza-s-indacene-3-yl)styryloxy)acetyl)aminohexanoicacid or succinimidyl ester; 4,4-difluoro-5-styryl-4-bora-3a, 4a-diaza-s-indacene-3-propionic acid; 4,4-difluoro-5-styryl-4-bora-3a,4a-diaza-sindacene-3-propionic acid; succinimidyl ester; 4,4-difluoro-1,3,5,7-tetramethyl-4-bora-3a,4adiaza-s-indacene-8-propionicacid; 4,4-difluoro-1,3,5,7-tetramethyl-4bora-3a,4a-diaza-sindacene-8-propionic acid succinimidyl ester; 4,4-difluoro-5-(2-thienyl)-4-bora-3a,4a-diaza-sindacene-3-propionic acid succinimidyl ester; 6-(((4-(4,4-difluoro-5-(2-thienyl)-4-bora-3a,4adiazas-indacene-3-yl)phenoxy)acetyl)amino)hexanoic acid or succinimidyl ester; and 6-(((4,4-difluoro-5-(2-thienyl)-4-bora-3a,4a-diaza-s-indacene-3-yl)styryloxy)acetyl)aminohexanoic acid or succinimidyl ester, Alexa Fluor®350 carboxylic acid; Alexa Fluor®430 carboxylic acid; Alexa Fluor®488 carboxylic acid; Alexa Fluor®532 carboxylic acid; Alexa Fluor®546 carboxylic acid; Alexa Fluor®555 carboxylic acid; Alexa Fluor®568 carboxylic acid; Alexa Fluor® 594 carboxylic acid; Alexa Fluor®633 carboxylic acid; Alexa Fluor®647 carboxylic acid; Alexa Fluor®660 carboxylic acid; Alexa Fluor®680 carboxylic acid, Cy3 NHS ester; Cy 5 NHS ester; Cy5.5 NHSester; and Cy7 NHS ester.

Embodiment 6. The composition of any one of Embodiments 1-5, wherein each fluorophore emits fluorescence at one, two, three, four, five, six, seven, eight, or nine wavelengths.

Embodiment 7. The composition of any one of Embodiments 1-6, wherein each fluorophore has a diameter of between about 500 nm and about 10 μm.

Embodiment 8. The composition of any one of Embodiments 1-7, wherein the polymer beads comprise less than 10%, 20%, 30%, or 40% polystyrene by hydrated volume.

Embodiment 8.1. The composition of any one of Embodiments 1-7, wherein the polymer beads comprise less than 10%, 20%, 30%, or 40% polystyrene by dehydrated volume.

Embodiment 9. The composition of any one of Embodiments 1-8.1, wherein the polymer beads are hydrogel beads.

Embodiment 10. The composition of Embodiment 9, wherein the hydrogel comprises a monomer.

Embodiment 11. The composition of Embodiment 10, wherein the monomer is hydroxyethyl methacrylate, ethyl methacrylate, 2-hydroxyethyl methacrylate (HEMA), propylene glycol methacrylate, acrylamide, N-vinylpyrrolidone (NVP), methyl methacrylate, glycidyl methacrylate, glycerol methacrylate (GMA), glycol methacrylate, ethylene glycol, fumaric acid, 2-hydroxyethyl methacrylate, hydroxyethoxyethyl methacrylate, hydroxydiethoxyethyl methacrylate, methoxyethyl methacrylate, methoxyethoxyethyl methacrylate, methoxydiethoxyethyl methacrylate, poly(ethylene glycol) methacrylate, methoxypoly(ethylene glycol) methacrylate, methacrylic acid, sodium methacrylate, glycerol methacrylate, hydroxypropyl methacrylate, hydroxybutyl methacrylate, phenyl acrylate, phenyl methacrylate, benzyl acrylate, benzyl methacrylate, 2-phenylethyl acrylate, 2-phenylethyl methacrylate, 2-phenoxyethyl acrylate, 2-phenoxyethyl methacrylate, phenylthioethyl acrylate, phenylthioethyl methacrylate, 2,4,6-tribromophenyl acrylate, 2,4,6-tribromophenyl methacrylate, pentabromophenyl acrylate, pentabromophenyl methacrylate, pentachlorophenyl acrylate, pentachlorophenyl methacrylate, 2,3-dibromopropyl acrylate, 2,3-dibromopropyl methacrylate, 2-naphthyl acrylate, 2-naphthyl methacrylate, 4-methoxybenzylacrylate, 4-methoxybenzyl methacrylate, 2-benzyloxyethyl acrylate, 2-benzyloxyethyl methacrylate, 4-chlorophenoxyethyl acrylate, 4-chlorophenoxyethyl methacrylate, 2-phenoxyethoxyethyl acrylate, 2-phenoxyethoxyethyl methacrylate, N-phenyl acrylamide, Nphenyl methacrylamide, N-benzyl acrylamide, N-benzyl methacrylamide, N,N-dibenzyl acrylamide, N,N-dibenzyl methacrylamide, N-diphenylmethyl acrylamide N-(4-methylphenyl)methyl acrylamide, N-1-naphthyl acrylamide, N-4-nitrophenyl acrylamide, N-(2-phenylethyl)acrylamide, N-triphenylmethyl acrylamide, N-(4-hydroxyphenyl)acrylamide, N,N-methylphenyl acrylamide, N,N-phenyl phenylethyl acrylamide, N-diphenylmethyl methacrylamide, N-(4-methyl phenyl)methyl methacrylamide, N-1-naphthyl methacrylamide, N-4-nitrophenyl methacrylamide, N-(2-phenylethyl)methacrylamide, N-triphenylmethyl methacrylamide, N-(4-hydroxyphenyl)methacrylamide, N,N-methylphenyl methacrylamide, N,N′-phenyl phenylethyl methacrylamide, N-vinylcarbazole, 4-vinylpyridine, 2-vinylpyridine, or a combination thereof.

Embodiment 12. The composition of any one of Embodiments 1-11, wherein the polymer beads exhibit at least one optical property that is substantially similar to the optical property of a target cell.

Embodiment 12.1. The composition of any one of Embodiments 1-11, at least one population of polymer beads exhibits at least one optical property that is distinct from the corresponding optical property of another population of polymer beads within the composition.

Embodiment 13. The composition of Embodiment 12 or 12.1, wherein the at least one optical property is side scatter.

Embodiment 14. The composition of Embodiment 12 or 12.1, wherein the at least one optical property is forward scatter.

Embodiment 15. The composition of Embodiment 12 or 12.1, wherein the at least one optical property comprises side scatter and forward scatter.

Embodiment 16. The composition of any one of Embodiments 12-15, wherein each target cell is independently selected from any one of: T cells, B cells, and natural killer cells.

Embodiment 17. The composition of any one of Embodiments 1-16, further comprising one or more of (iii) a third population of polymer beads comprising a third fluorophore, (iv) a fourth population of polymer beads comprising a fourth fluorophore, (v) a fifth population of polymer beads comprising a a fifth fluorophore, and/or (vi) a sixth population of polymer beads comprising a a sixth fluorophore.

Embodiment 17.1. The composition of Embodiment 17, wherein each fluorophore is independently selected from those listed in Table 4.

Embodiment 18. The composition of Embodiment 17, wherein the first, second, third, fourth, fifth, and sixth fluorophores are independently selected from the group consisting of: peridinin chlorophyll protein-cyanine 5.5 dye (PerCP-Cy5.5); phycoerythrin-cyanine7 (PE Cy7); allophycocyanin-cyanine 7 (APC-Cy7); fluorescein isothiocyanate (FITC); phycoerythrin (PE); allophyscocyanin (APC); 6-carboxy-4′,5′-dichloro-2′,7′-dimethoxyfluorescein succinimidylester; 5-(and-6)-carboxyeosin; 5-carboxyfluorescein; 6 carboxyfluorescein; 5-(and-6)-carboxyfluorescein; S-carboxyfluorescein-bis-(5-carboxymethoxy-2-nitrobenzyl)ether,-alanine-carboxamide, or succinimidyl ester; 5-carboxy fluorescein succinimidyl ester; 6-carboxyfluorescein succinimidyl ester; 5-(and-6)-carboxyfluorescein succinimidyl ester; 5-(4,6-dichlorotriazinyl)amino fluorescein; 2′,7′-difluoro fluorescein; eosin-5-isothiocyanate; erythrosin5-isothiocyanate; 6-(fluorescein-5-carboxamido) hexanoic acid or succinimidyl ester; 6-(fluorescein-5-(and-6)-carboxamido) hexanoic acid or succinimidylester; fluorescein-S-EX succinimidyl ester; fluorescein-5-isothiocyanate; fluorescein-6-isothiocyanate; OregonGreen® 488 carboxylic acid, or succinimidyl ester; Oregon Green® 488 isothiocyanate; Oregon Green® 488-X succinimidyl ester; Oregon Green® 500 carboxylic acid; Oregon Green® 500 carboxylic acid, succinimidylester or triethylammonium salt; Oregon Green® 514 carboxylic acid; Oregon Green® 514 carboxylic acid or succinimidyl ester; RhodamineGreen™ carboxylic acid, succinimidyl ester or hydrochloride; Rhodamine Green™ carboxylic acid, trifluoroacetamide or succinimidylester; Rhodamine Green™-X succinimidyl ester or hydrochloride; RhodolGreen™ carboxylic acid, N,O-bis-(trifluoroacetyl) or succinimidylester; bis-(4-carboxypiperidinyl) sulfonerhodamine or di(succinimidylester); 5-(and-6)carboxynaphtho fluorescein, 5-(and-6)carboxynaphthofluorescein succinimidyl ester; 5-carboxyrhodamine 6G hydrochloride; 6-carboxyrhodamine6Ghydrochloride, 5-carboxyrhodamine 6G succinimidyl ester; 6-carboxyrhodamine 6G succinimidyl ester; 5-(and-6)-carboxyrhodamine6G succinimidyl ester; 5-carboxy-2′,4′,5′,7′-tetrabromosulfonefluorescein succinimidyl esteror bis-(diisopropylethylammonium) salt; 5-carboxytetramethylrhodamine; 6-carboxytetramethylrhodamine; 5-(and-6)-carboxytetramethylrhodamine; 5-carboxytetramethylrhodamine succinimidyl ester; 6-carboxytetramethylrhodaminesuccinimidyl ester; 5-(and-6)-carboxytetramethylrhodamine succinimidyl ester; 6-carboxy-X-rhodamine; 5-carboxy-X-rhodamine succinimidyl ester; 6-carboxy-X-rhodamine succinimidyl ester; 5-(and-6)-carboxy-X-rhodamine succinimidyl ester; 5-carboxy-X-rhodamine triethylammonium salt; Lissamine™ rhodamine B sulfonyl chloride; malachite green; isothiocyanate; NANOGOLD® mono(sulfosuccinimidyl ester); QSY® 21carboxylic acid or succinimidyl ester; QSY® 7 carboxylic acid or succinimidyl ester; Rhodamine Red™-X succinimidyl ester; 6-(tetramethylrhodamine-5-(and-6)-carboxamido) hexanoic acid; succinimidyl ester; tetramethylrhodamine-5-isothiocyanate; tetramethylrhodamine-6-isothiocyanate; tetramethylrhodamine-5-(and-6)-isothiocyanate; Texas Red® sulfonyl; Texas Red® sulfonyl chloride; Texas Red®-X STP ester or sodium salt; Texas Red®-X succinimidyl ester; Texas Red®-X succinimidyl ester; X-rhodamine-5-(and-6) isothiocyanate, BODIPY® FL; BODIPY® TMR STP ester; BODIPY® TR-X STP ester; BODIPY® 630/650-X STPester; BODIPY® 650/665-X STP ester; 6-dibromo-4,4-difluoro-5,7-dimethyl-4-bora-3a,4a-diaza-s-indacene-3-propionic acid succinimidyl ester; 4,4-difluoro-4-bora-3a,4a-diaza-s-indacene-3,5-dipropionic acid; 4,4-difluoro-5,7-dimethyl-4-bora-3a,4a-diaza-s-indacene-3-pentanoicacid; 4,4-difluoro-5,7-dimethyl-4-bora3a,4a-diaza-s-indacene-3-pentanoicacid succinimidyl ester; 4,4-difluoro-5,7-dimefhyl-4-bora-3a,4a-diaza-s-indacene-3propionicacid; 4,4-difluoro-5,7-dimethyl-4-bora-3a,4adiaza-s-indacene-3-propionicacid succinimidyl ester; 4,4-difluoro-5,7-dimefhyl-4-bora-3a,4a-diaza-s-indacene-3propionic acid; sulfosuccinimidyl ester or sodium salt; 6-((4,4-difluoro-5,7-dimethyl-4-bora-3a,4a-diaza-s-indacene-3propionyl)amino)hexanoicacid; 6-((4,4-difluoro-5,7 dimethyl-4-bora-3a,4a-diaza-s-indacene-3-propionyl)amino)hexanoic acid or succinimidyl ester; N-(4,4-difluoro 5,7-dimethyl-4-bora-3a,4a-diaza-s-indacene-3-propionyl) cysteic acid, succinimidyl ester or triethylammonium salt; 6-4,4-difluoro-1,3-dimethyl-5-(4-methoxyphenyl)-4-bora3a,4a4,4-difluoro-5,7-diphenyl-4-bora-3a,4a-diaza-sindacene-3-propionicacid; 4,4-difluoro-5,7-diphenyl-4-bora3a,4a-diaza-s-indacene-3-propionicacid succinimidyl ester; 4,4-difluoro-5-phenyl-4-bora-3a,4a-diaza-s-indacene-3-propionic acid; succinimidyl ester; 6-((4,4-difluoro-5-phenyl-4 bora-3a,4a-diaza-s-indacene-3-propionyl)amino) hexanoicacid or succinimidyl ester; 4,4-difluoro-5-(4-phenyl-1,3butadienyl)-4-bora-3a,4a-diaza-s-indacene-3-propionicacid succinimidyl ester; 4,4-difluoro-5-(2-pyrrolyl)-4-bora-3a,4a-diaza-s-indacene-3-propionic acid succinimidyl ester; 6-(((4,4-difluoro-5-(2-pyrrolyl)-4-bora-3a,4a-diaza-s-indacene-3-yl)styryloxy)acetyl)aminohexanoicacid or succinimidyl ester; 4,4-difluoro-5-styryl-4-bora-3a, 4a-diaza-s-indacene-3-propionic acid; 4,4-difluoro-5-styryl-4-bora-3a,4a-diaza-sindacene-3-propionic acid; succinimidyl ester; 4,4-difluoro-1,3,5,7-tetramethyl-4-bora-3a,4adiaza-s-indacene-8-propionicacid; 4,4-difluoro-1,3,5,7-tetramethyl-4bora-3a,4a-diaza-sindacene-8-propionic acid succinimidyl ester; 4,4-difluoro-5-(2-thienyl)-4-bora-3a,4a-diaza-sindacene-3-propionic acid succinimidyl ester; 6-(((4-(4,4-difluoro-5-(2-thienyl)-4-bora-3a,4adiazas-indacene-3-yl)phenoxy)acetyl)amino)hexanoic acid or succinimidyl ester; and 6-(((4,4-difluoro-5-(2-thienyl)-4-bora-3a,4a-diaza-s-indacene-3-yl)styryloxy)acetyl)aminohexanoic acid or succinimidyl ester, Alexa Fluor®350 carboxylic acid; Alexa Fluor®430 carboxylic acid; Alexa Fluor®488 carboxylic acid; Alexa Fluor®532 carboxylic acid; Alexa Fluor®546 carboxylic acid; Alexa Fluor®555 carboxylic acid; Alexa Fluor® 568 carboxylic acid; Alexa Fluor® 594 carboxylic acid; Alexa Fluor®633 carboxylic acid; Alexa Fluor®647 carboxylic acid; Alexa Fluor®660 carboxylic acid; Alexa Fluor®680 carboxylic acid, Cy3 NHS ester; Cy 5 NHS ester; Cy5.5 NHSester; and Cy7 NHS ester.

Embodiment 18.1. The composition of any one of Embodiments 1-18, wherein the fluorophores are conjugated to an antibody or fragment thereof that is bound to an epitope within the polymer beads.

Embodiment 18.2. The composition of Embodiment 18.1, wherein the epitope is a biomarker comprised in the polymer beads.

Embodiment 18.3. The composition of Embodiment 18.1 or 18.2, wherein the fluorophore is a commercially-available antibody-label conjugate.

Embodiment 18.4. The composition of Embodiment 18.2 or 18.3, wherein the biomarker is selected from those listed in Tables 1-3 of this specification.

Embodiment 19. A method of calibrating a cytometric device for compensation or spectral unmixing comprising (i) measuring a fluorescence signal of a composition of any one of Embodiments 1-18.3 using the cytometric device, (ii) deconvoluting the fluorescence signal from each polymer bead population of the composition to calculate a compensation or spectral unmixing matrix, and (iii) calibrating the cytometric device using the compensation or spectral unmixing matrix.

Embodiment 19.1. A method of calibrating a cytometric device for compensation or spectral unmixing comprising (A) measuring, using the cytometric device, a fluorescence signal of a composition comprising (i) a first population of polymer beads comprising a first fluorophore and (ii) a second population of polymer beads comprising a second fluorophore, (B) deconvoluting the fluorescence signal from each polymer bead population of the composition to calculate a compensation or spectral unmixing matrix, and (C) calibrating the cytometric device using the compensation or spectral unmixing matrix.

Embodiment 19.2. The method of Embodiment 19 or 19.1, wherein the fluorescence signal form each polymer bead population is deconvoluted based on fluorescence emission maximas.

Embodiment 19.3. The method of Embodiment 19 or 19.1, wherein the fluorescence signal form each polymer bead population is deconvoluted based on optical properties of each population of polymer beads.

Embodiment 19.4. The method of any one of Embodiments 19-19.3 wherein the measured composition comprises up to 5, up to 10, up to 12, up to 18, up to 20, up to 30, up to 40, up to 50, up to 60, up to 70, up to 80, up to 90, or up to 100 populations of polymer beads, wherein each bead population comprises a fluorophore, and wherein the fluorophore for each population of beads is different.

Embodiment 19.5. The method of any one of Embodiments 19-19.4, wherein the first fluorophore and the second fluorophore are different fluorophores.

Embodiment 19.6. The method of any one of Embodiments 19-19.4, wherein each population of polymer beads comprises only a single fluorophore.

Embodiment 19.7. The method of any one of Embodiments 19-19.6, wherein the measured composition comprises a final population of polymer beads that do not comprise any fluorophores.

Embodiment 19.7.1. The method of any one of Embodiments 19.19.6, wherein each fluorophore is independently selected from those listed in Table 4.

Embodiment 19.8. The method of any one of Embodiments 19-19.7, wherein each fluorophore is independently selected from any one of: peridinin chlorophyll protein-cyanine 5.5 dye (PerCP-Cy5.5); phycoerythrin-cyanine7 (PE Cy7); allophycocyanin-cyanine 7 (APC-Cy7); fluorescein isothiocyanate (FITC); phycoerythrin (PE); allophyscocyanin (APC); 6-carboxy-4′,5′-dichloro-2′,7-dimethoxyfluorescein succinimidylester; 5-(and-6)-carboxyeosin; 5-carboxyfluorescein; 6 carboxyfluorescein; 5-(and-6)-carboxyfluorescein; S-carboxyfluorescein-bis-(5-carboxymethoxy-2-nitrobenzyl)ether,-alanine-carboxamide, or succinimidyl ester; 5-carboxy fluorescein succinimidyl ester; 6-carboxyfluorescein succinimidyl ester; 5-(and-6)-carboxyfluorescein succinimidyl ester; 5-(4,6-dichlorotriazinyl)amino fluorescein; 2′,7-difluoro fluorescein; eosin-5-isothiocyanate; erythrosin5-isothiocyanate; 6-(fluorescein-5-carboxamido) hexanoic acid or succinimidyl ester; 6-(fluorescein-5-(and-6)-carboxamido) hexanoic acid or succinimidylester; fluorescein-S-EX succinimidyl ester; fluorescein-5-isothiocyanate; fluorescein-6-isothiocyanate; OregonGreen® 488 carboxylic acid, or succinimidyl ester; Oregon Green® 488 isothiocyanate; Oregon Green® 488-X succinimidyl ester; Oregon Green® 500 carboxylic acid; Oregon Green® 500 carboxylic acid, succinimidylester or triethylammonium salt; Oregon Green® 514 carboxylic acid; Oregon Green® 514 carboxylic acid or succinimidyl ester; RhodamineGreen™ carboxylic acid, succinimidyl ester or hydrochloride; Rhodamine Green™ carboxylic acid, trifluoroacetamide or succinimidylester; Rhodamine Green™-X succinimidyl ester or hydrochloride; RhodolGreen™ carboxylic acid, N,O-bis-(trifluoroacetyl) or succinimidylester; bis-(4-carboxypiperidinyl) sulfonerhodamine or di(succinimidylester); 5-(and-6)carboxynaphtho fluorescein, 5-(and-6)carboxynaphthofluorescein succinimidyl ester; 5-carboxyrhodamine 6G hydrochloride; 6-carboxyrhodamine6Ghydrochloride, 5-carboxyrhodamine 6G succinimidyl ester; 6-carboxyrhodamine 6G succinimidyl ester; 5-(and-6)-carboxyrhodamine6G succinimidyl ester; 5-carboxy-2′,4′,5′,7′-tetrabromosulfonefluorescein succinimidyl esteror bis-(diisopropylethylammonium) salt; 5-carboxytetramethylrhodamine; 6-carboxytetramethylrhodamine; 5-(and-6)-carboxytetramethylrhodamine; 5-carboxytetramethylrhodamine succinimidyl ester; 6-carboxytetramethylrhodaminesuccinimidyl ester; 5-(and-6)-carboxytetramethylrhodamine succinimidyl ester; 6-carboxy-X-rhodamine; 5-carboxy-X-rhodamine succinimidyl ester; 6-carboxy-X-rhodamine succinimidyl ester; 5-(and-6)-carboxy-X-rhodamine succinimidyl ester; 5-carboxy-X-rhodamine triethylammonium salt; Lissamine™ rhodamine B sulfonyl chloride; malachite green; isothiocyanate; NANOGOLD® mono(sulfosuccinimidyl ester); QSY® 21carboxylic acid or succinimidyl ester; QSY® 7 carboxylic acid or succinimidyl ester; Rhodamine Red™-X succinimidyl ester; 6-(tetramethylrhodamine-5-(and-6)-carboxamido) hexanoic acid; succinimidyl ester; tetramethylrhodamine-5-isothiocyanate; tetramethylrhodamine-6-isothiocyanate; tetramethylrhodamine-5-(and-6)-isothiocyanate; Texas Red® sulfonyl; Texas Red® sulfonyl chloride; Texas Red®-X STP ester or sodium salt; Texas Red®-X succinimidyl ester; Texas Red®-X succinimidyl ester; X-rhodamine-5-(and-6) isothiocyanate, BODIPY® FL; BODIPY® TMR STP ester; BODIPY® TR-X STP ester; BODIPY® 630/650-X STPester; BODIPY® 650/665-X STP ester; 6-dibromo-4,4-difluoro-5,7-dimethyl-4-bora-3a,4a-diaza-s-indacene-3-propionic acid succinimidyl ester; 4,4-difluoro-4-bora-3a,4a-diaza-s-indacene-3,5-dipropionic acid; 4,4-difluoro-5,7-dimethyl-4-bora-3a,4a-diaza-s-indacene-3-pentanoicacid; 4,4-difluoro-5,7-dimethyl-4-bora3a,4a-diaza-s-indacene-3-pentanoicacid succinimidyl ester; 4,4-difluoro-5,7-dimefhyl-4-bora-3a,4a-diaza-s-indacene-3propionicacid; 4,4-difluoro-5,7-dimethyl-4-bora-3a,4adiaza-s-indacene-3-propionicacid succinimidyl ester; 4,4-difluoro-5,7-dimefhyl-4-bora-3a,4a-diaza-s-indacene-3propionic acid; sulfosuccinimidyl ester or sodium salt; 6-((4,4-difluoro-5,7-dimethyl-4-bora-3a,4a-diaza-s-indacene-3propionyl)amino)hexanoicacid; 6-((4,4-difluoro-5,7 dimethyl-4-bora-3a,4a-diaza-s-indacene-3-propionyl)amino)hexanoic acid or succinimidyl ester; N-(4,4-difluoro 5,7-dimethyl-4-bora-3a,4a-diaza-s-indacene-3-propionyl) cysteic acid, succinimidyl ester or triethylammonium salt; 6-4,4-difluoro-1,3-dimethyl-5-(4-methoxyphenyl)-4-bora3a,4a4,4-difluoro-5,7-diphenyl-4-bora-3a,4a-diaza-sindacene-3-propionicacid; 4,4-difluoro-5,7-diphenyl-4-bora3a,4a-diaza-s-indacene-3-propionicacid succinimidyl ester; 4,4-difluoro-5-phenyl-4-bora-3a,4a-diaza-s-indacene-3-propionic acid; succinimidyl ester; 6-((4,4-difluoro-5-phenyl-4 bora-3a,4a-diaza-s-indacene-3-propionyl)amino) hexanoicacid or succinimidyl ester; 4,4-difluoro-5-(4-phenyl-1,3butadienyl)-4-bora-3a,4a-diaza-s-indacene-3-propionicacid succinimidyl ester; 4,4-difluoro-5-(2-pyrrolyl)-4-bora-3a,4a-diaza-s-indacene-3-propionic acid succinimidyl ester; 6-(((4,4-difluoro-5-(2-pyrrolyl)-4-bora-3a,4a-diaza-s-indacene-3-yl)styryloxy)acetyl)aminohexanoicacid or succinimidyl ester; 4,4-difluoro-5-styryl-4-bora-3a, 4a-diaza-s-indacene-3-propionic acid; 4,4-difluoro-5-styryl-4-bora-3a,4a-diaza-sindacene-3-propionic acid; succinimidyl ester; 4,4-difluoro-1,3,5,7-tetramethyl-4-bora-3a,4adiaza-s-indacene-8-propionicacid; 4,4-difluoro-1,3,5,7-tetramethyl-4bora-3a,4a-diaza-sindacene-8-propionic acid succinimidyl ester; 4,4-difluoro-5-(2-thienyl)-4-bora-3a,4a-diaza-sindacene-3-propionic acid succinimidyl ester; 6-(((4-(4,4-difluoro-5-(2-thienyl)-4-bora-3a,4adiazas-indacene-3-yl)phenoxy)acetyl)amino)hexanoic acid or succinimidyl ester; and 6-(((4,4-difluoro-5-(2-thienyl)-4-bora-3a,4a-diaza-s-indacene-3-yl)styryloxy)acetyl)aminohexanoic acid or succinimidyl ester, Alexa Fluor®350 carboxylic acid; Alexa Fluor®430 carboxylic acid; Alexa Fluor®488 carboxylic acid; Alexa Fluor®532 carboxylic acid; Alexa Fluor®546 carboxylic acid; Alexa Fluor®555 carboxylic acid; Alexa Fluor®568 carboxylic acid; Alexa Fluor® 594 carboxylic acid; Alexa Fluor®633 carboxylic acid; Alexa Fluor®647 carboxylic acid; Alexa Fluor®660 carboxylic acid; Alexa Fluor®680 carboxylic acid, Cy3 NHS ester; Cy 5 NHS ester; Cy5.5 NHSester; and Cy7 NHS ester.

Embodiment 19.9. The method of any one of Embodiments 19-19.8, wherein each fluorophore emits fluorescence at one, two, three, four, five, six, seven, eight, or nine wavelengths.

Embodiment 19.10. The method of any one of Embodiments 19-19.9, wherein each fluorophore has a diameter of between about 500 nm and about 10 μm.

Embodiment 19.11. The method of any one of Embodiments 19-19.10, wherein the polymer beads comprise less than 10%, 20%, 30%, 40% polystyrene by hydrated volume.

Embodiment 19.11.1. The method of any one of Embodiments 19-19.10, wherein the polymer beads comprise less than 10%, 20%, 30%, 40% polystyrene by dehydrated volume.

Embodiment 19.12. The method of any one of Embodiments 19-19.11.1, wherein the polymer beads are hydrogel beads.

Embodiment 19.13. The method of Embodiment 19.12, wherein the hydrogel comprises a monomer.

Embodiment 19.14. The method of Embodiment 19.13, wherein the monomer is hydroxyethyl methacrylate, ethyl methacrylate, 2-hydroxyethyl methacrylate (HEMA), propylene glycol methacrylate, acrylamide, N-vinylpyrrolidone (NVP), methyl methacrylate, glycidyl methacrylate, glycerol methacrylate (GMA), glycol methacrylate, ethylene glycol, fumaric acid, 2-hydroxyethyl methacrylate, hydroxyethoxyethyl methacrylate, hydroxydiethoxyethyl methacrylate, methoxyethyl methacrylate, methoxyethoxyethyl methacrylate, methoxydiethoxyethyl methacrylate, poly(ethylene glycol) methacrylate, methoxypoly(ethylene glycol) methacrylate, methacrylic acid, sodium methacrylate, glycerol methacrylate, hydroxypropyl methacrylate, hydroxybutyl methacrylate, phenyl acrylate, phenyl methacrylate, benzyl acrylate, benzyl methacrylate, 2-phenylethyl acrylate, 2-phenylethyl methacrylate, 2-phenoxyethyl acrylate, 2-phenoxyethyl methacrylate, phenylthioethyl acrylate, phenylthioethyl methacrylate, 2,4,6-tribromophenyl acrylate, 2,4,6-tribromophenyl methacrylate, pentabromophenyl acrylate, pentabromophenyl methacrylate, pentachlorophenyl acrylate, pentachlorophenyl methacrylate, 2,3-dibromopropyl acrylate, 2,3-dibromopropyl methacrylate, 2-naphthyl acrylate, 2-naphthyl methacrylate, 4-methoxybenzylacrylate, 4-methoxybenzyl methacrylate, 2-benzyloxyethyl acrylate, 2-benzyloxyethyl methacrylate, 4-chlorophenoxyethyl acrylate, 4-chlorophenoxyethyl methacrylate, 2-phenoxyethoxyethyl acrylate, 2-phenoxyethoxyethyl methacrylate, N-phenyl acrylamide, Nphenyl methacrylamide, N-benzyl acrylamide, N-benzyl methacrylamide, N,N-dibenzyl acrylamide, N,N-dibenzyl methacrylamide, N-diphenylmethyl acrylamide N-(4-methylphenyl)methyl acrylamide, N-1-naphthyl acrylamide, N-4-nitrophenyl acrylamide, N-(2-phenylethyl)acrylamide, N-triphenylmethyl acrylamide, N-(4-hydroxyphenyl)acrylamide, N,N-methylphenyl acrylamide, N,N-phenyl phenylethyl acrylamide, N-diphenylmethyl methacrylamide, N-(4-methyl phenyl)methyl methacrylamide, N-1-naphthyl methacrylamide, N-4-nitrophenyl methacrylamide, N-(2-phenylethyl)methacrylamide, N-triphenylmethyl methacrylamide, N-(4-hydroxyphenyl)methacrylamide, N,N-methylphenyl methacrylamide, N,N′-phenyl phenylethyl methacrylamide, N-vinylcarbazole, 4-vinylpyridine, 2-vinylpyridine, or a combination thereof.

Embodiment 19.15. The method of any one of Embodiments 19-19.7, wherein the polymer beads exhibit at least one optical property that is substantially similar to the optical property of a target cell.

Embodiment 19.16. The method of any one of Embodiments 19-19.15, wherein at least one population of polymer beads exhibits at least one optical property that is distinct from the corresponding optical property of another population of polymer beads within the composition.

Embodiment 19.17. The method of Embodiment 19.15 or 19.16, wherein the at least one optical property is side scatter.

Embodiment 19.18. The method of Embodiment 19.15 or 19.16, wherein the at least one optical property is forward scatter.

Embodiment 19.19. The method of Embodiment 19.15 or 19.16, wherein the at least one optical property comprises side scatter and forward scatter.

Embodiment 19.20. The method of any one of Embodiments 19.15 and 19.17-19.19, wherein each target cell is independently selected from any one of: T cells, B cells, and natural killer cells.

Embodiment 19.21. The method of any one of Embodiments 19.1-19.20, wherein the measured composition comprises one or more of (iii) a third population of polymer beads comprising a third fluorophore, (iv) a fourth population of polymer beads comprising a fourth fluorophore, (v) a fifth population of polymer beads comprising a a fifth fluorophore, and/or (vi) a sixth population of polymer beads comprising a a sixth fluorophore.

Embodiment 19.22. The method of Embodiment 19.2, wherein the first, second, third, fourth, fifth, and sixth fluorophores are independently selected from the group consisting of: PerCP-Cy5.5, PE Cy7, APC-Cy7, FITC, PE, and APC.

Embodiment 20. A method of calibrating a cytometric device for compensation or spectral unmixing comprising (A) providing a composition comprising (i) a first population of polymer beads comprising a first fluorophore and (ii) a second population of polymer beads comprising a second fluorophore, (B) measuring a fluorescence signal of the composition using the cytometric device, (C) deconvoluting the fluorescence signal from each bead population of the composition to calculate a compensation or spectral unmixing matrix, and (D) calibrating the cytometric device using the compensation or spectral unmixing matrix.

Embodiment 20.1. The method of any one of Embodiments 19-20, wherein each population of polymer beads contains sufficiently high contents of fluorophore so as to create a fluorescence signal that is at least as strong as a fluorescent signal from a cell population to be analyzed via the cytometric device.

Embodiment 21. A composition comprising (i) a first population of polymer beads comprising a first biomarker, and (ii) a second population of polymer beads comprising a second biomarker.

Embodiment 22. The composition of Embodiment 21, comprising up to 5, up to 10, up to 12, up to 18, up to 20, up to 30, up to 40, up to 50, up to 60, up to 70, up to 80, up to 90, or up to 100 populations of polymer beads, wherein each bead population comprises a biomarker, and wherein the biomarker for each bead population is different.

Embodiment 23. The composition of Embodiment 21 or 22, wherein each population of polymer beads comprises a different biomarker.

Embodiment 23.1. The composition of any one of Embodiments 21-23, wherein each population of polymer beads comprises only a single biomarker.

Embodiment 24. The composition of any one of Embodiments 21-23.1, comprising a population of beads that does not comprise a fluorophore.

Embodiment 25. The composition of any one of Embodiments 21-24, comprising a population of beads that does not comprise a biomarker.

Embodiment 26. The composition of any one of Embodiments 21-25, wherein the polymer beads comprise less than 10%, 20%, 30%, or 40% polystyrene by hydrated volume.

Embodiment 26.1. The composition of any one of Embodiments 21-25, wherein the polymer beads comprise less than 10%, 20%, 30%, or 40% polystyrene by dehydrated volume.

Embodiment 27. The composition of any one of Embodiments 21-26, wherein the polymer beads are hydrogel beads.

Embodiment 27.1. The composition of any one of Embodiments 21-27, wherein the biomarker is a polypeptide.

Embodiment 27.2. The composition of any one of Embodiments 21-27.1, wherein the biomarker is an epitope for a fluorescent dye.

Embodiment 27.3. The composition of any one of Embodiments 21-27.2, wherein each the first and second population of polymer beads each comprise a different fluorophore.

Embodiment 27.4. The composition of any one of Embodiments 21-27.3, wherein the biomarker is an epitope for an antibody.

Embodiment 27.5. The composition of Embodiment 27.4, wherein the antibody is configured to bind to a fluorescent dye or is configured to bind to a secondary antibody-fluorophore conjugate.

Embodiment 27.6. The composition of any one of Embodiments 21-27.5, wherein the fluorophores are conjugated to an antibody or fragment thereof, that is bound to an epitope within the polymer beads.

Embodiment 27.7. The composition of Embodiment 27.6, wherein the epitope is the biomarker comprised in the polymer beads.

Embodiment 27.8. The composition of Embodiment 27.6 or 27.7, wherein the fluorophore is a commercially-available antibody-label conjugate.

Embodiment 27.9. The composition of any one of Embodiments 27.6-27.8, wherein the biomarker is selected from those listed in Tables 1-3 of this specification.

Embodiment 28. The composition of any one of Embodiments 21-27.2, wherein each biomarker is independently selected from any one of: CD3, CD4, CD8, CD19, CD14, ccr7, CD45, CD45RA, CD27, CD16, CD56, CD127, CD25, CD38, HLA-DR, PD-1, CD28, CD183, CD185, CD57, IFN-gamma, CD20, TCR gamma/delta, TNF alpha, CD69, IL-2, Ki-67, CCR6, CD34, CD45RO, CD161, IgD, CD95, CD117, CD123, CD11c, IgM, CD39, FoxP3, CD10, CD40L, CD62L, CD194, CD314, IgG, TCR V alpha 7.2, CD11b, CD21, CD24, IL-4, Biotin, CCR10, CD31, CD44, CD138, CD294, NKp46, TCR V delta 2, TIGIT, CD1c, CD2, CD7, CD8a, CD15, CD32, CD103, CD107a, CD141, CD158, CD159c, IL-13, IL-21, KLRG1, TIM-3, CCR5, CD5, CD33, CD45.2, CD80, CD159a (NKG2a), CD244, CD272, CD278, CD337, Granzyme B, Ig Lambda Light Chain, IgA, IL-17A, Streptavidin, TCR V delta 1, CD1d, CD26, CD45R (B220), CD64, CD73, CD86, CD94, CD137, CD163, CD193, CTLA-4, CX3CR1, Fc epsilon R1 alpha, IL-22, Lag-3, MIP-1 beta, Perforin, TCR V gamma 9, CD1a, CD22, CD36, CD40, CD45R, CD66b, CD85j, CD160, CD172a, CD186, CD226, CD303, CLEC12A, CXCR4, Helios, Ig Kappa Light Chain, IgE, IgG1, IgG3, IL-5, IL-8, IL-21 R, KIR3d105, KLRC1/2, Ly-6C, Ly-6G, MHC Class II (I-A/I-E), MHC II, TCR alpha/beta, TCR beta, TCR V alpha 24, Akt (pS473), ALDH1A1, Annexin V, Bcl-2, c-Met, CCR7, cd16/32, cd41a, CD3 epsilon, CD8b, CD11b/c, CD16/CD32, CD23, CD29, CD43, CD45.1, CD48, CD49b, CD49d, CD66, CD68, CD71, CD85k, CD93, CD99, CD106, CD122, CD133, CD134, CD146, CD150, CD158b, CD158b1/b2, j, CD158e, CD166, CD169, CD184, CD200, CD200 R, CD235a, CD267, CD268, CD273, CD274, CD317, CD324, CD326, CD328, CD336, CD357, CD366, DDR2, eFluor 780 Fix Viability, EGF Receptor, EGFR (pY845), EOMES, EphA2, ERK1/2 (pT202/pY204), F4/80, FCRL5, Flt-3, FVS575V, FVS700, Granzyme A, HER2/ErbB2, Hes1, Hoechst (33342), ICAM-1, IFN-alpha, IgA1, IgA1/IgA2, IgA2, IgG2, IgG4, IL-1 RAcP, IL-6, IL-10, IL-12, IL-17, Integrin alpha 4 beta 7, Isotype Ctrl, KLRC1, KLRC2, Live/Dead Fix Aqua, Ly-6A/Ly-6E, Ly-6G/Ly-6C, Mannose Receptor, MDR1, Met (pY1234/pY1235), MMP-9, NGF Receptor p75, ORAI1, ORAI2, ORAI3, p53, P2RY12, PARP, cleaved, RT1B, S6 (pS235/pS236), STIM1, STIM2, TCR delta, TCR delta/gamma, TCR V alpha 24 J alpha 18, TCR V beta 11, TCR V gamma 1.1, TCR V gamma 2, TER-119, TIMP-3, TRAF3, TSLP Receptor, VDAC1, Vimentin, XCR1, and YAP1.

Embodiment 29. The composition of any one of Embodiments 27-28, wherein the hydrogel comprises polyacrylamide.

Embodiment 30. The composition of any one of Embodiments 21-29, comprising (iii) a third population of beads comprising a third biomarker, (iv) a fourth population of beads comprising a fourth biomarker, (v) a fifth population of beads comprising a fifth biomarker, and (vi) a sixth population of beads comprising a sixth biomarker.

Embodiment 31. The composition of Embodiment 30, wherein the first, second, third, fourth, fifth, and sixth biomarkers are independently selected from the group consisting of: CD3, CD16, CD56, CD45, CD4, CD19, and CD8.

Embodiment 32. The composition of Embodiment 30 or 31, wherein each polymer bead population comprises a different fluorophore.

Embodiment 33. The composition of Embodiment 27, wherein the hydrogel comprises a matrix comprising a monomer.

Embodiment 34. The composition of Embodiment 33, wherein the monomer is hydroxyethyl methacrylate, ethyl methacrylate, 2-hydroxyethyl methacrylate (HEMA), propylene glycol methacrylate, acrylamide, N-vinylpyrrolidone (NVP), methyl methacrylate, glycidyl methacrylate, glycerol methacrylate (GMA), glycol methacrylate, ethylene glycol, fumaric acid, 2-hydroxyethyl methacrylate, hydroxyethoxyethyl methacrylate, hydroxydiethoxyethyl methacrylate, methoxyethyl methacrylate, methoxyethoxyethyl methacrylate, methoxydiethoxyethyl methacrylate, poly(ethylene glycol) methacrylate, methoxypoly(ethylene glycol) methacrylate, methacrylic acid, sodium methacrylate, glycerol methacrylate, hydroxypropyl methacrylate, hydroxybutyl methacrylate, phenyl acrylate, phenyl methacrylate, benzyl acrylate, benzyl methacrylate, 2-phenylethyl acrylate, 2-phenylethyl methacrylate, 2-phenoxyethyl acrylate, 2-phenoxyethyl methacrylate, phenylthioethyl acrylate, phenylthioethyl methacrylate, 2,4,6-tribromophenyl acrylate, 2,4,6-tribromophenyl methacrylate, pentabromophenyl acrylate, pentabromophenyl methacrylate, pentachlorophenyl acrylate, pentachlorophenyl methacrylate, 2,3-dibromopropyl acrylate, 2,3-dibromopropyl methacrylate, 2-naphthyl acrylate, 2-naphthyl methacrylate, 4-methoxybenzylacrylate, 4-methoxybenzyl methacrylate, 2-benzyloxyethyl acrylate, 2-benzyloxyethyl methacrylate, 4-chlorophenoxyethyl acrylate, 4-chlorophenoxyethyl methacrylate, 2-phenoxyethoxyethyl acrylate, 2-phenoxyethoxyethyl methacrylate, N-phenyl acrylamide, Nphenyl methacrylamide, N-benzyl acrylamide, N-benzyl methacrylamide, N,N-dibenzyl acrylamide, N,N-dibenzyl methacrylamide, N-diphenylmethyl acrylamide N-(4-methylphenyl)methyl acrylamide, N-1-naphthyl acrylamide, N-4-nitrophenyl acrylamide, N-(2-phenylethyl)acrylamide, N-triphenylmethyl acrylamide, N-(4-hydroxyphenyl)acrylamide, N,N-methylphenyl acrylamide, N,N-phenyl phenylethyl acrylamide, N-diphenylmethyl methacrylamide, N-(4-methyl phenyl)methyl methacrylamide, N-1-naphthyl methacrylamide, N-4-nitrophenyl methacrylamide, N-(2-phenylethyl)methacrylamide, N-triphenylmethyl methacrylamide, N-(4-hydroxyphenyl)methacrylamide, N,N-methylphenyl methacrylamide, N,N′-phenyl phenylethyl methacrylamide, N-vinylcarbazole, 4-vinylpyridine, 2-vinylpyridine, or a combination thereof.

Embodiment 35. The composition of any one of Embodiments 21-34, wherein the polymer beads exhibit at least one optical property that is substantially similar to the optical property of a target cell.

Embodiment 35.1. The composition of any one of Embodiments 21-34, at least one population of polymer beads exhibits at least one optical property that is distinct from the corresponding optical property of another population of polymer beads within the composition.

Embodiment 36. The composition of Embodiment 35 or 35.1, wherein the at least one optical property is side scatter.

Embodiment 37. The composition of Embodiment 35 or 35.1, wherein the at least one optical property is forward scatter.

Embodiment 38. The composition of Embodiment 35 or 35.1, wherein the at least one optical property comprises side scatter and forward scatter.

Embodiment 39. The composition of Embodiment 35, wherein each target cell is independently selected from any one of: T cells, B cells, and natural killer cells.

Embodiment 40. A method of calibrating a cytometric device for compensation or spectral unmixing comprising (i) measuring a fluorescence signal of a composition of any one of Embodiments 21-39 using the cytometric device, (ii) deconvoluting the fluorescence signal from each bead population of the composition to calculate a compensation or spectral unmixing matrix, and (iii) calibrating the cytometric device using the compensation or spectral unmixing matrix.

Embodiment 40.1. A method of calibrating a cytometric device for compensation or spectral unmixing comprising (A) measuring, using the cytometric device, a fluorescence signal of a composition comprising (i) a first population of polymer beads comprising a first biomarker and (ii) a second population of polymer beads comprising a second biomarker, (B) deconvoluting the fluorescence signal from each bead population of the composition to calculate a compensation or spectral unmixing matrix, and (C) calibrating the cytometric device using the compensation or spectral unmixing matrix.

Embodiment 40.2. The method of Embodiment 40.1, wherein each population of polymer beads comprises a different fluorophore.

Embodiment 41. A method of producing a cytometric device multi-color compensation control, said method comprising the steps of (A) contacting a composition comprising (i) a first population of polymer beads comprising a first biomarker and (ii) a second population of polymer beads comprising a second biomarker with a plurality of fluorescent dyes in a single reaction, wherein each dye in the plurality of fluorescent dyes comprises a fluorophore with a different excitation or emission spectra from the fluorophore in every other dye in the plurality of fluorescent dyes, and wherein each dye binds to the biomarker of only a single population of polymer beads in the composition, such that each population of polymer beads is bound to no more than one fluorescent dye, thereby producing a multi-color compensation control.

Embodiment 41.1. The method of any one of Embodiments 40.1-41, wherein each biomarker of the populations of polymer beads comprises an antigen configured to selectively bind to a fluorescent dye in.

Embodiment 41.2. The method of any one of Embodiment 41-41.1, wherein the plurality of fluorescent dyes are secondary antibody-fluorophore conjugates, and wherein each biomarker of the populations of polymer beads comprises an antigen configured to selectively bind to a secondary antibody-fluorophore conjugate.

Embodiment 42. The method of any one of Embodiments 41-41.2, wherein each dye in the plurality of fluorescent dyes comprises an antibody-fluorophore conjugate.

Embodiment 42.1. The method of any one of Embodiments 41-42, wherein each fluorophore is independently selected from those listed in Table 4.

Embodiment 43. The method of any one of Embodiments 40.1-42.1, wherein each fluorophore is independently selected from any one of: peridinin chlorophyll protein-cyanine 5.5 dye (PerCP-Cy5.5); phycoerythrin-cyanine7 (PE Cy7); allophycocyanin-cyanine 7 (APC-Cy7); fluorescein isothiocyanate (FITC); phycoerythrin (PE); allophyscocyanin (APC); 6-carboxy-4′,5′-dichloro-2′,7-dimethoxyfluorescein succinimidylester; 5-(and-6)-carboxyeosin; 5-carboxyfluorescein; 6 carboxyfluorescein; 5-(and-6)-carboxyfluorescein; S-carboxyfluorescein-bis-(5-carboxymethoxy-2-nitrobenzyl)ether,-alanine-carboxamide, or succinimidyl ester; 5-carboxy fluorescein succinimidyl ester; 6-carboxyfluorescein succinimidyl ester; 5-(and-6)-carboxyfluorescein succinimidyl ester; 5-(4,6-dichlorotriazinyl)amino fluorescein; 2′,7-difluoro fluorescein; eosin-5-isothiocyanate; erythrosin5-isothiocyanate; 6-(fluorescein-5-carboxamido) hexanoic acid or succinimidyl ester; 6-(fluorescein-5-(and-6)-carboxamido) hexanoic acid or succinimidylester; fluorescein-S-EX succinimidyl ester; fluorescein-5-isothiocyanate; fluorescein-6-isothiocyanate; OregonGreen® 488 carboxylic acid, or succinimidyl ester; Oregon Green® 488 isothiocyanate; Oregon Green® 488-X succinimidyl ester; Oregon Green® 500 carboxylic acid; Oregon Green® 500 carboxylic acid, succinimidylester or triethylammonium salt; Oregon Green® 514 carboxylic acid; Oregon Green® 514 carboxylic acid or succinimidyl ester; RhodamineGreen™ carboxylic acid, succinimidyl ester or hydrochloride; Rhodamine Green™ carboxylic acid, trifluoroacetamide or succinimidylester; Rhodamine Green™-X succinimidyl ester or hydrochloride; RhodolGreen™ carboxylic acid, N,O-bis-(trifluoroacetyl) or succinimidylester; bis-(4-carboxypiperidinyl) sulfonerhodamine or di(succinimidylester); 5-(and-6)carboxynaphtho fluorescein, 5-(and-6)carboxynaphthofluorescein succinimidyl ester; 5-carboxyrhodamine 6G hydrochloride; 6-carboxyrhodamine6Ghydrochloride, 5-carboxyrhodamine 6G succinimidyl ester; 6-carboxyrhodamine 6G succinimidyl ester; 5-(and-6)-carboxyrhodamine6G succinimidyl ester; 5-carboxy-2′,4′,5′,7′-tetrabromosulfonefluorescein succinimidyl esteror bis-(diisopropylethylammonium) salt; 5-carboxytetramethylrhodamine; 6-carboxytetramethylrhodamine; 5-(and-6)-carboxytetramethylrhodamine; 5-carboxytetramethyirhodamine succinimidyl ester; 6-carboxytetramethylrhodaminesuccinimidyl ester; 5-(and-6)-carboxytetramethylrhodamine succinimidyl ester; 6-carboxy-X-rhodamine; 5-carboxy-X-rhodamine succinimidyl ester; 6-carboxy-X-rhodamine succinimidyl ester; 5-(and-6)-carboxy-X-rhodamine succinimidyl ester; 5-carboxy-X-rhodamine triethylammonium salt; Lissamine™ rhodamine B sulfonyl chloride; malachite green; isothiocyanate; NANOGOLD® mono(sulfosuccinimidyl ester); QSY® 21carboxylic acid or succinimidyl ester; QSY® 7 carboxylic acid or succinimidyl ester; Rhodamine Red™-X succinimidyl ester; 6-(tetramethylrhodamine-5-(and-6)-carboxamido) hexanoic acid; succinimidyl ester; tetramethylrhodamine-5-isothiocyanate; tetramethylrhodamine-6-isothiocyanate; tetramethylrhodamine-5-(and-6)-isothiocyanate; Texas Red® sulfonyl; Texas Red® sulfonyl chloride; Texas Red®-X STP ester or sodium salt; Texas Red®-X succinimidyl ester; Texas Red®-X succinimidyl ester; X-rhodamine-5-(and-6) isothiocyanate, BODIPY® FL; BODIPY® TMR STP ester; BODIPY® TR-X STP ester; BODIPY® 630/650-X STPester; BODIPY® 650/665-X STP ester; 6-dibromo-4,4-difluoro-5,7-dimethyl-4-bora-3a,4a-diaza-s-indacene-3-propionic acid succinimidyl ester; 4,4-difluoro-4-bora-3a,4a-diaza-s-indacene-3,5-dipropionic acid; 4,4-difluoro-5,7-dimethyl-4-bora-3a,4a-diaza-s-indacene-3-pentanoicacid; 4,4-difluoro-5,7-dimethyl-4-bora3a,4a-diaza-s-indacene-3-pentanoicacid succinimidyl ester; 4,4-difluoro-5,7-dimefhyl-4-bora-3a,4a-diaza-s-indacene-3propionicacid; 4,4-difluoro-5,7-dimethyl-4-bora-3a,4adiaza-s-indacene-3-propionicacid succinimidyl ester; 4,4-difluoro-5,7-dimefhyl-4-bora-3a,4a-diaza-s-indacene-3propionic acid; sulfosuccinimidyl ester or sodium salt; 6-((4,4-difluoro-5,7-dimethyl-4-bora-3a,4a-diaza-s-indacene-3propionyl)amino)hexanoicacid; 6-((4,4-difluoro-5,7 dimethyl-4-bora-3a,4a-diaza-s-indacene-3-propionyl)amino)hexanoic acid or succinimidyl ester; N-(4,4-difluoro 5,7-dimethyl-4-bora-3a,4a-diaza-s-indacene-3-propionyl) cysteic acid, succinimidyl ester or triethylammonium salt; 6-4,4-difluoro-1,3-dimethyl-5-(4-methoxyphenyl)-4-bora3a,4a4,4-difluoro-5,7-diphenyl-4-bora-3a,4a-diaza-sindacene-3-propionicacid; 4,4-difluoro-5,7-diphenyl-4-bora3a,4a-diaza-s-indacene-3-propionicacid succinimidyl ester; 4,4-difluoro-5-phenyl-4-bora-3a,4a-diaza-s-indacene-3-propionic acid; succinimidyl ester; 6-((4,4-difluoro-5-phenyl-4 bora-3a,4a-diaza-s-indacene-3-propionyl)amino) hexanoicacid or succinimidyl ester; 4,4-difluoro-5-(4-phenyl-1,3butadienyl)-4-bora-3a,4a-diaza-s-indacene-3-propionicacid succinimidyl ester; 4,4-difluoro-5-(2-pyrrolyl)-4-bora-3a,4a-diaza-s-indacene-3-propionic acid succinimidyl ester; 6-(((4,4-difluoro-5-(2-pyrrolyl)-4-bora-3a,4a-diaza-s-indacene-3-yl)styryloxy)acetyl)aminohexanoicacid or succinimidyl ester; 4,4-difluoro-5-styryl-4-bora-3a, 4a-diaza-s-indacene-3-propionic acid; 4,4-difluoro-5-styryl-4-bora-3a,4a-diaza-sindacene-3-propionic acid; succinimidyl ester; 4,4-difluoro-1,3,5,7-tetramethyl-4-bora-3a,4adiaza-s-indacene-8-propionicacid; 4,4-difluoro-1,3,5,7-tetramethyl-4bora-3a,4a-diaza-sindacene-8-propionic acid succinimidyl ester; 4,4-difluoro-5-(2-thienyl)-4-bora-3a,4a-diaza-sindacene-3-propionic acid succinimidyl ester; 6-(((4-(4,4-difluoro-5-(2-thienyl)-4-bora-3a,4adiazas-indacene-3-yl)phenoxy)acetyl)amino)hexanoic acid or succinimidyl ester; and 6-(((4,4-difluoro-5-(2-thienyl)-4-bora-3a,4a-diaza-s-indacene-3-yl)styryloxy)acetyl)aminohexanoic acid or succinimidyl ester, Alexa Fluor®350 carboxylic acid; Alexa Fluor®430 carboxylic acid; Alexa Fluor®488 carboxylic acid; Alexa Fluor®532 carboxylic acid; Alexa Fluor®546 carboxylic acid; Alexa Fluor®555 carboxylic acid; Alexa Fluor®568 carboxylic acid; Alexa Fluor® 594 carboxylic acid; Alexa Fluor®633 carboxylic acid; Alexa Fluor®647 carboxylic acid; Alexa Fluor®660 carboxylic acid; Alexa Fluor®680 carboxylic acid, Cy3 NHS ester; Cy 5 NHS ester; Cy5.5 NHSester; and Cy7 NHS ester.

Embodiment 44. The method of any one of Embodiments 40.1-43, wherein the composition comprises up to 5, up to 10, up to 12, up to 18, up to 20, up to 30, up to 40, up to 50, up to 60, up to 70, up to 80, up to 90, or up to 100 populations of polymer beads, wherein each bead population comprises a biomarker, and wherein the biomarker for each bead population is different.

Embodiment 45. The method of any one of Embodiments 40.1-44, wherein each population of polymer beads comprises a different biomarker.

Embodiment 46. The method of any one of Embodiments 40.1-45, wherein each population of polymer beads comprises only a single biomarker.

Embodiment 47. The method of any one of Embodiments 40.1-46, comprising a population of beads that does not comprise a fluorophore or lacks the epitope for associating with an antibody-fluorophore conjugates.

Embodiment 48. The method of any one of Embodiments 40.1-47, comprising a population of beads that does not comprise a biomarker.

Embodiment 49. The method of any one of Embodiments 40.1-48, wherein the polymer beads comprise less than 10%, 20%, 30%, or 40% polystyrene by hydrated volume.

Embodiment 50. The method of any one of Embodiments 40.1-48, wherein the polymer beads comprise less than 10%, 20%, 30%, or 40% polystyrene by dehydrated volume.

Embodiment 51. The method of any one of Embodiments 40.1-50, wherein the polymer beads are hydrogel beads.

Embodiment 52. The method of any one of Embodiments 40.1-51, wherein the biomarker is a polypeptide.

Embodiment 53. The method of any one of Embodiments 40.1-52, wherein the biomarker is an epitope for a fluorescent dye.

Embodiment 54. The method of any one of Embodiments 40.1-53, wherein each the first and second population of polymer beads each comprise a different fluorophore.

Embodiment 55. The method of any one of Embodiments 40.1-54, wherein the biomarker is an epitope for an antibody.

Embodiment 56. The method of any one of Embodiments 40.1-55, wherein the antibody is configured to bind to a fluorescent dye or is configured to bind to a secondary antibody-fluorophore conjugate.

Embodiment 57. The method of any one of Embodiments 40.1-56, wherein the fluorophores are conjugated to an antibody or fragment thereof, that is bound to an epitope within the polymer beads.

Embodiment 58. The method of Embodiment 57, wherein the epitope is the biomarker comprised in the polymer beads.

Embodiment 59. The method of any one of Embodiments 40.1-58, wherein the fluorophore is a commercially-available antibody-label conjugate.

Embodiment 60. The method of any one of Embodiments 40.1-59, wherein the biomarker is selected from those listed in Tables 1-3 of this specification.

Embodiment 61. The method of any one of Embodiments 40.1-59, wherein each biomarker is independently selected from any one of: CD3, CD4, CD8, CD19, CD14, ccr7, CD45, CD45RA, CD27, CD16, CD56, CD127, CD25, CD38, HLA-DR, PD-1, CD28, CD183, CD185, CD57, IFN-gamma, CD20, TCR gamma/delta, TNF alpha, CD69, IL-2, Ki-67, CCR6, CD34, CD45RO, CD161, IgD, CD95, CD117, CD123, CD11c, IgM, CD39, FoxP3, CD10, CD40L, CD62L, CD194, CD314, IgG, TCR V alpha 7.2, CD11b, CD21, CD24, IL-4, Biotin, CCR10, CD31, CD44, CD138, CD294, NKp46, TCR V delta 2, TIGIT, CD1c, CD2, CD7, CD8a, CD15, CD32, CD103, CD107a, CD141, CD158, CD159c, IL-13, IL-21, KLRG1, TIM-3, CCR5, CD5, CD33, CD45.2, CD80, CD159a (NKG2a), CD244, CD272, CD278, CD337, Granzyme B, Ig Lambda Light Chain, IgA, IL-17A, Streptavidin, TCR V delta 1, CD1d, CD26, CD45R (B220), CD64, CD73, CD86, CD94, CD137, CD163, CD193, CTLA-4, CX3CR1, Fc epsilon R1 alpha, IL-22, Lag-3, MIP-1 beta, Perforin, TCR V gamma 9, CD1a, CD22, CD36, CD40, CD45R, CD66b, CD85j, CD160, CD172a, CD186, CD226, CD303, CLEC12A, CXCR4, Helios, Ig Kappa Light Chain, IgE, IgG1, IgG3, IL-5, IL-8, IL-21 R, KIR3d105, KLRC1/2, Ly-6C, Ly-6G, MHC Class II (I-A/I-E), MHC II, TCR alpha/beta, TCR beta, TCR V alpha 24, Akt (pS473), ALDH1A1, Annexin V, Bcl-2, c-Met, CCR7, cd16/32, cd41a, CD3 epsilon, CD8b, CD11b/c, CD16/CD32, CD23, CD29, CD43, CD45.1, CD48, CD49b, CD49d, CD66, CD68, CD71, CD85k, CD93, CD99, CD106, CD122, CD133, CD134, CD146, CD150, CD158b, CD158b1/b2, j, CD158e, CD166, CD169, CD184, CD200, CD200 R, CD235a, CD267, CD268, CD273, CD274, CD317, CD324, CD326, CD328, CD336, CD357, CD366, DDR2, eFluor 780 Fix Viability, EGF Receptor, EGFR (pY845), EOMES, EphA2, ERK1/2 (pT202/pY204), F4/80, FCRL5, Flt-3, FVS575V, FVS700, Granzyme A, HER2/ErbB2, Hes1, Hoechst (33342), ICAM-1, IFN-alpha, IgA1, IgA1/IgA2, IgA2, IgG2, IgG4, IL-1 RAcP, IL-6, IL-10, IL-12, IL-17, Integrin alpha 4 beta 7, Isotype Ctrl, KLRC1, KLRC2, Live/Dead Fix Aqua, Ly-6A/Ly-6E, Ly-6G/Ly-6C, Mannose Receptor, MDR1, Met (pY1234/pY1235), MMP-9, NGF Receptor p75, ORAI1, ORAI2, ORAI3, p53, P2RY12, PARP, cleaved, RT1B, S6 (pS235/pS236), STIM1, STIM2, TCR delta, TCR delta/gamma, TCR V alpha 24 J alpha 18, TCR V beta 11, TCR V gamma 1.1, TCR V gamma 2, TER-119, TIMP-3, TRAF3, TSLP Receptor, VDAC1, Vimentin, XCR1, and YAP1.

Embodiment 62. The method of any one of Embodiments 51-61, wherein the hydrogel comprises polyacrylamide.

Embodiment 63. The method of any one of Embodiments 40.1-62, comprising (iii) a third population of beads comprising a third biomarker, (iv) a fourth population of beads comprising a fourth biomarker, (v) a fifth population of beads comprising a fifth biomarker, and (vi) a sixth population of beads comprising a sixth biomarker.

Embodiment 64. The method of Embodiment 63, wherein the first, second, third, fourth, fifth, and sixth biomarkers are independently selected from the group consisting of: CD3, CD16, CD56, CD45, CD4, CD19, and CD8.

Embodiment 65. The method of any one of Embodiments 40.1-64, wherein each polymer bead population comprises a different fluorophore.

Embodiment 66. The method of any one of Embodiments 51-65, wherein the hydrogel comprises a matrix comprising a monomer.

Embodiment 67. The method of Embodiment 66, wherein the monomer is hydroxyethyl methacrylate, ethyl methacrylate, 2-hydroxyethyl methacrylate (HEMA), propylene glycol methacrylate, acrylamide, N-vinylpyrrolidone (NVP), methyl methacrylate, glycidyl methacrylate, glycerol methacrylate (GMA), glycol methacrylate, ethylene glycol, fumaric acid, 2-hydroxyethyl methacrylate, hydroxyethoxyethyl methacrylate, hydroxydiethoxyethyl methacrylate, methoxyethyl methacrylate, methoxyethoxyethyl methacrylate, methoxydiethoxyethyl methacrylate, poly(ethylene glycol) methacrylate, methoxypoly(ethylene glycol) methacrylate, methacrylic acid, sodium methacrylate, glycerol methacrylate, hydroxypropyl methacrylate, hydroxybutyl methacrylate, phenyl acrylate, phenyl methacrylate, benzyl acrylate, benzyl methacrylate, 2-phenylethyl acrylate, 2-phenylethyl methacrylate, 2-phenoxyethyl acrylate, 2-phenoxyethyl methacrylate, phenylthioethyl acrylate, phenylthioethyl methacrylate, 2,4,6-tribromophenyl acrylate, 2,4,6-tribromophenyl methacrylate, pentabromophenyl acrylate, pentabromophenyl methacrylate, pentachlorophenyl acrylate, pentachlorophenyl methacrylate, 2,3-dibromopropyl acrylate, 2,3-dibromopropyl methacrylate, 2-naphthyl acrylate, 2-naphthyl methacrylate, 4-methoxybenzylacrylate, 4-methoxybenzyl methacrylate, 2-benzyloxyethyl acrylate, 2-benzyloxyethyl methacrylate, 4-chlorophenoxyethyl acrylate, 4-chlorophenoxyethyl methacrylate, 2-phenoxyethoxyethyl acrylate, 2-phenoxyethoxyethyl methacrylate, N-phenyl acrylamide, Nphenyl methacrylamide, N-benzyl acrylamide, N-benzyl methacrylamide, N,N-dibenzyl acrylamide, N,N-dibenzyl methacrylamide, N-diphenylmethyl acrylamide N-(4-methylphenyl)methyl acrylamide, N-1-naphthyl acrylamide, N-4-nitrophenyl acrylamide, N-(2-phenylethyl)acrylamide, N-triphenylmethyl acrylamide, N-(4-hydroxyphenyl)acrylamide, N,N-methylphenyl acrylamide, N,N-phenyl phenylethyl acrylamide, N-diphenylmethyl methacrylamide, N-(4-methyl phenyl)methyl methacrylamide, N-1-naphthyl methacrylamide, N-4-nitrophenyl methacrylamide, N-(2-phenylethyl)methacrylamide, N-triphenylmethyl methacrylamide, N-(4-hydroxyphenyl)methacrylamide, N,N-methylphenyl methacrylamide, N,N′-phenyl phenylethyl methacrylamide, N-vinylcarbazole, 4-vinylpyridine, 2-vinylpyridine, or a combination thereof.

Embodiment 68. The method of any one of Embodiments 40.1-67, wherein the polymer beads exhibit at least one optical property that is substantially similar to the optical property of a target cell.

Embodiment 69. The method of any one of Embodiments 40.1-68, at least one population of polymer beads exhibits at least one optical property that is distinct from the corresponding optical property of another population of polymer beads within the composition.

Embodiment 70. The method of Embodiment 68 or 69, wherein the at least one optical property is side scatter.

Embodiment 71. The method of any one of Embodiments 68-69, wherein the at least one optical property is forward scatter.

Embodiment 72. The method of any one of Embodiments 68-69, wherein the at least one optical property comprises side scatter and forward scatter.

Embodiment 73. The method of any one of Embodiments 40.1-72, wherein each target cell is independently selected from any one of: T cells, B cells, and natural killer cells.

Embodiment 74. A method of calibrating a cytometric device for compensation or spectral unmixing comprising (i) providing a composition comprising (a) a first population of polymer beads comprising a first biomarker and (b) a second population of polymer beads comprising a second biomarker, each of the first and second population of polymer beads comprising a different fluorophore, (ii) contacting the composition with at least two population of antibodies or fragments thereof, each population of antibodies capable of binding to only one of the biomarkers in the composition, wherein the antibodies or fragments thereof are conjugated to a fluorophore capable of generating a fluorescent signal, (iii) measuring a fluorescence signal of the composition using the cytometric device, (iv) deconvoluting the fluorescence signal from each bead population of the composition to calculate a compensation or spectral unmixing matrix, and (v) calibrating the cytometric device using the compensation or spectral unmixing matrix.

Embodiment 75. The method of any one of Embodiments 40-74, wherein the fluorescence signal form each polymer bead population is deconvoluted based on fluorescence emission maximas.

Embodiment 76. The method of any one of Embodiments 40-74, wherein the fluorescence signal form each polymer bead population is deconvoluted based on optical properties of each population of polymer beads.

All, documents, patents, patent applications, publications, product descriptions, and protocols which are cited throughout this application are incorporated herein by reference in their entireties for all purposes. This document explicitly incorporates the following U.S. and PCT patent applications in their entireties for all purposes: US 2022/0178810; US 2020/0400546; US 2021/0341469; US 2021/0231552; US 2020/0400546; PCT/US2023/06668; and PCT/US2023/067893.

The embodiments illustrated and discussed in this specification are intended only to teach those skilled in the art the best way known to the inventors to make and use inventions of the present disclosure. Modifications and variation of the above-described embodiments of the present disclosure are possible without departing from the spirit of the inventions, as appreciated by those skilled in the art in light of the above teachings. It is therefore understood that, within the scope of the claims and their equivalents, inventions of the present disclosure may be practiced otherwise than as specifically described.

1. A method of calibrating a cytometric device for compensation or spectral unmixing comprising:
(i) measuring a fluorescence signal of a composition comprising: (a) a first population of polymer beads comprising a first fluorophore; and (b) a second population of polymer beads comprising a second fluorophore using the cytometric device;
(ii) deconvoluting the fluorescence signal from each polymer bead population of the composition;
(iii) calculating a compensation or spectral unmixing matrix; and
(iv) calibrating the cytometric device using the compensation or spectral unmixing matrix;

2. The method of claim 1, wherein the first population of polymer beads and the second population of polymer beads each independently exhibit at least one optical property that is substantially similar to the optical property of a target cell.

3. The method of claim 2, comprising at least one population of polymer beads that exhibits at least one optical property that is distinct from the corresponding optical property of another population of polymer beads within the composition.

4. The method of claim 2, wherein the at least one optical property is side scatter.

5. The method of claim 2, wherein the at least one optical property is forward scatter.

6. The method of claim 1, comprising:
(v) inserting a biological cell into the cytometric device, wherein the biological cell comprises the first fluorophore and/or the second fluorophore tethered to the cell.

7. The method of claim 1, wherein calculating the compensation or spectral unmixing matrix comprises performing an Ordinary Least Square calculation.

8. The method of claim 1, wherein the first fluorophore and/or the second fluorophore are tethered to a biomarker on the surface of a hydrogel particle.

9. The method of claim 1, comprising deconvoluting the fluorescence signal from each polymer bead population based on the fluorescence emission maximum of each population, an optical property of each population, or a combination thereof.

10. The method of claim 1, wherein the first fluorophore and the second fluorophore of the composition are different fluorophores.

11. The method of claim 1, wherein each population of polymer beads of the composition comprises only a single fluorophore.

12. The method of claim 1, wherein the composition comprises a third population of polymer beads that does not comprise a fluorophore.

13. The method of claim 1, wherein each fluorophore of the composition is independently selected from any one of: peridinin chlorophyll protein-cyanine 5.5 dye (PerCP-Cy5.5); phycoerythrin-cyanine7 (PE Cy7); allophycocyanin-cyanine 7 (APC-Cy7); fluorescein isothiocyanate (FITC); phycoerythrin (PE); allophyscocyanin (APC); 6-carboxy-4′,5′-dichloro-2′,7-dimethoxyfluorescein succinimidylester; 5-(and-6)-carboxyeosin; 5-carboxyfluorescein; 6 carboxyfluorescein; 5-(and-6)-carboxyfluorescein; S-carboxyfluorescein-bis-(5-carboxymethoxy-2-nitrobenzyl)ether,-alanine-carboxamide, or succinimidyl ester; 5-carboxy fluorescein succinimidyl ester; 6-carboxyfluorescein succinimidyl ester; 5-(and-6)-carboxyfluorescein succinimidyl ester; 5-(4,6-dichlorotriazinyl)amino fluorescein; 2′,7-difluoro fluorescein; eosin-5-isothiocyanate; erythrosin5-isothiocyanate; 6-(fluorescein-5-carboxamido) hexanoic acid or succinimidyl ester; 6-(fluorescein-5-(and-6)-carboxamido) hexanoic acid or succinimidylester; fluorescein-S-EX succinimidyl ester; fluorescein-5-isothiocyanate; fluorescein-6-isothiocyanate; OregonGreen® 488 carboxylic acid, or succinimidyl ester; Oregon Green® 488 isothiocyanate; Oregon Green® 488-X succinimidyl ester; Oregon Green® 500 carboxylic acid; Oregon Green® 500 carboxylic acid, succinimidylester or triethylammonium salt; Oregon Green® 514 carboxylic acid; Oregon Green® 514 carboxylic acid or succinimidyl ester; RhodamineGreen™ carboxylic acid, succinimidyl ester or hydrochloride; Rhodamine Green™ carboxylic acid, trifluoroacetamide or succinimidylester; Rhodamine Green™-X succinimidyl ester or hydrochloride; RhodolGreen™ carboxylic acid, N,O-bis-(trifluoroacetyl) or succinimidylester; bis-(4-carboxypiperidinyl) sulfonerhodamine or di(succinimidylester); 5-(and-6)carboxynaphtho fluorescein, 5-(and-6)carboxynaphthofluorescein succinimidyl ester; 5-carboxyrhodamine 6G hydrochloride; 6-carboxyrhodamine6Ghydrochloride, 5-carboxyrhodamine 6G succinimidyl ester; 6-carboxyrhodamine 6G succinimidyl ester; 5-(and-6)-carboxyrhodamine6G succinimidyl ester; 5-carboxy-2′,4′,5′,7′-tetrabromosulfonefluorescein succinimidyl esteror bis-(diisopropylethylammonium) salt; 5-carboxytetramethylrhodamine; 6-carboxytetramethylrhodamine; 5-(and-6)-carboxytetramethylrhodamine; 5-carboxytetramethylrhodamine succinimidyl ester; 6-carboxytetramethylrhodaminesuccinimidyl ester; 5-(and-6)-carboxytetramethylrhodamine succinimidyl ester; 6-carboxy-X-rhodamine; 5-carboxy-X-rhodamine succinimidyl ester; 6-carboxy-X-rhodamine succinimidyl ester; 5-(and-6)-carboxy-X-rhodamine succinimidyl ester; 5-carboxy-X-rhodamine triethylammonium salt; Lissamine™ rhodamine B sulfonyl chloride; malachite green; isothiocyanate; NANOGOLD® mono(sulfosuccinimidyl ester); QSY® 21carboxylic acid or succinimidyl ester; QSY® 7 carboxylic acid or succinimidyl ester; Rhodamine Red™-X succinimidyl ester; 6-(tetramethylrhodamine-5-(and-6)-carboxamido) hexanoic acid; succinimidyl ester; tetramethylrhodamine-5-isothiocyanate; tetramethylrhodamine-6-isothiocyanate; tetramethylrhodamine-5-(and-6)-isothiocyanate; Texas Red® sulfonyl; Texas Red® sulfonyl chloride; Texas Red®-X STP ester or sodium salt; Texas Red®-X succinimidyl ester; Texas Red®-X succinimidyl ester; X-rhodamine-5-(and-6) isothiocyanate, BODIPY® FL; BODIPY® TMR STP ester; BODIPY® TR-X STP ester; BODIPY® 630/650-X STPester; BODIPY® 650/665-X STP ester; 6-dibromo-4,4-difluoro-5,7-dimethyl-4-bora-3a,4a-diaza-s-indacene-3-propionic acid succinimidyl ester; 4,4-difluoro-4-bora-3a,4a-diaza-s-indacene-3,5-dipropionic acid; 4,4-difluoro-5,7-dimethyl-4-bora-3a,4a-diaza-s-indacene-3-pentanoicacid; 4,4-difluoro-5,7-dimethyl-4-bora3a,4a-diaza-s-indacene-3-pentanoicacid succinimidyl ester; 4,4-difluoro-5,7-dimefhyl-4-bora-3a,4a-diaza-s-indacene-3propionicacid; 4,4-difluoro-5,7-dimethyl-4-bora-3a,4adiaza-s-indacene-3-propionicacid succinimidyl ester; 4,4-difluoro-5,7-dimefhyl-4-bora-3a,4a-diaza-s-indacene-3propionic acid; sulfosuccinimidyl ester or sodium salt; 6-((4,4-difluoro-5,7-dimethyl-4-bora-3a,4a-diaza-s-indacene-3propionyl)amino)hexanoicacid; 6-((4,4-difluoro-5,7 dimethyl-4-bora-3a,4a-diaza-s-indacene-3-propionyl)amino)hexanoic acid or succinimidyl ester; N-(4,4-difluoro 5,7-dimethyl-4-bora-3a,4a-diaza-s-indacene-3-propionyl) cysteic acid, succinimidyl ester or triethylammonium salt; 6-4,4-difluoro-1,3-dimethyl-5-(4-methoxyphenyl)-4-bora3a,4a4,4-difluoro-5,7-diphenyl-4-bora-3a,4a-diaza-sindacene-3-propionicacid; 4,4-difluoro-5,7-diphenyl-4-bora3a,4a-diaza-s-indacene-3-propionicacid succinimidyl ester; 4,4-difluoro-5-phenyl-4-bora-3a,4a-diaza-s-indacene-3-propionic acid; succinimidyl ester; 6-((4,4-difluoro-5-phenyl-4 bora-3a,4a-diaza-s-indacene-3-propionyl)amino) hexanoicacid or succinimidyl ester; 4,4-difluoro-5-(4-phenyl-1,3butadienyl)-4-bora-3a,4a-diaza-s-indacene-3-propionicacid succinimidyl ester; 4,4-difluoro-5-(2-pyrrolyl)-4-bora-3a,4a-diaza-s-indacene-3-propionic acid succinimidyl ester; 6-(((4,4-difluoro-5-(2-pyrrolyl)-4-bora-3a,4a-diaza-s-indacene-3-yl)styryloxy)acetyl)aminohexanoicacid or succinimidyl ester; 4,4-difluoro-5-styryl-4-bora-3a, 4a-diaza-s-indacene-3-propionic acid; 4,4-difluoro-5-styryl-4-bora-3a,4a-diaza-sindacene-3-propionic acid; succinimidyl ester; 4,4-difluoro-1,3,5,7-tetramethyl-4-bora-3a,4adiaza-s-indacene-8-propionicacid; 4,4-difluoro-1,3,5,7-tetramethyl-4bora-3a,4a-diaza-sindacene-8-propionic acid succinimidyl ester; 4,4-difluoro-5-(2-thienyl)-4-bora-3a,4a-diaza-sindacene-3-propionic acid succinimidyl ester; 6-(((4-(4,4-difluoro-5-(2-thienyl)-4-bora-3a,4adiazas-indacene-3-yl)phenoxy)acetyl)amino)hexanoic acid or succinimidyl ester; and 6-(((4,4-difluoro-5-(2-thienyl)-4-bora-3a,4a-diaza-s-indacene-3-yl)styryloxy)acetyl)aminohexanoic acid or succinimidyl ester, Alexa Fluor® 350 carboxylic acid; Alexa Fluor® 430 carboxylic acid; Alexa Fluor® 488 carboxylic acid; Alexa Fluor® 532 carboxylic acid; Alexa Fluor® 546 carboxylic acid; Alexa Fluor® 555 carboxylic acid; Alexa Fluor® 568 carboxylic acid; Alexa Fluor® 594 carboxylic acid; Alexa Fluor® 633 carboxylic acid; Alexa Fluor® 647 carboxylic acid; Alexa Fluor® 660 carboxylic acid; Alexa Fluor® 680 carboxylic acid, Cy3 NHS ester; Cy 5 NHS ester; Cy5.5 NHSester; and Cy7 NHS ester.

14. The method of claim 1, wherein the polymer beads in the composition are hydrogel beads.

15. The method of claim 11, wherein the hydrogel beads comprise a monomer.

16. The method of claim 15, wherein the monomer is hydroxyethyl methacrylate, ethyl methacrylate, 2-hydroxyethyl methacrylate (HEMA), propylene glycol methacrylate, acrylamide, N-vinylpyrrolidone (NVP), methyl methacrylate, glycidyl methacrylate, glycerol methacrylate (GMA), glycol methacrylate, ethylene glycol, fumaric acid, 2-hydroxyethyl methacrylate, hydroxyethoxyethyl methacrylate, hydroxydiethoxyethyl methacrylate, methoxyethyl methacrylate, methoxyethoxyethyl methacrylate, methoxydiethoxyethyl methacrylate, poly(ethylene glycol) methacrylate, methoxypoly(ethylene glycol) methacrylate, methacrylic acid, sodium methacrylate, glycerol methacrylate, hydroxypropyl methacrylate, hydroxybutyl methacrylate, phenyl acrylate, phenyl methacrylate, benzyl acrylate, benzyl methacrylate, 2-phenylethyl acrylate, 2-phenylethyl methacrylate, 2-phenoxyethyl acrylate, 2-phenoxyethyl methacrylate, phenylthioethyl acrylate, phenylthioethyl methacrylate, 2,4,6-tribromophenyl acrylate, 2,4,6-tribromophenyl methacrylate, pentabromophenyl acrylate, pentabromophenyl methacrylate, pentachlorophenyl acrylate, pentachlorophenyl methacrylate, 2,3-dibromopropyl acrylate, 2,3-dibromopropyl methacrylate, 2-naphthyl acrylate, 2-naphthyl methacrylate, 4-methoxybenzylacrylate, 4-methoxybenzyl methacrylate, 2-benzyloxyethyl acrylate, 2-benzyloxyethyl methacrylate, 4-chlorophenoxyethyl acrylate, 4-chlorophenoxyethyl methacrylate, 2-phenoxyethoxyethyl acrylate, 2-phenoxyethoxyethyl methacrylate, N-phenyl acrylamide, Nphenyl methacrylamide, N-benzyl acrylamide, N-benzyl methacrylamide, N,N-dibenzyl acrylamide, N,N-dibenzyl methacrylamide, N-diphenylmethyl acrylamide N-(4-methylphenyl)methyl acrylamide, N-1-naphthyl acrylamide, N-4-nitrophenyl acrylamide, N-(2-phenylethyl)acrylamide, N-triphenylmethyl acrylamide, N-(4-hydroxyphenyl)acrylamide, N,N-methylphenyl acrylamide, N,N-phenyl phenylethyl acrylamide, N-diphenylmethyl methacrylamide, N-(4-methyl phenyl)methyl methacrylamide, N-1-naphthyl methacrylamide, N-4-nitrophenyl methacrylamide, N-(2-phenylethyl)methacrylamide, N-triphenylmethyl methacrylamide, N-(4-hydroxyphenyl)methacrylamide, N,N-methylphenyl methacrylamide, N,N′-phenyl phenylethyl methacrylamide, N-vinylcarbazole, 4-vinylpyridine, 2-vinylpyridine, or a combination thereof.

17. The method of claim 1, wherein the composition comprises:
(c) a third population of polymer beads comprising a third fluorophore;
(d) a fourth population of polymer beads comprising a fourth fluorophore;
(e) a fifth population of polymer beads comprising a fifth fluorophore; and/or
(f) a sixth population of polymer beads comprising a sixth fluorophore.

18. The method of claim 17, wherein the composition comprises:
(g) a seventh population of polymer beads that does not comprise a fluorophore.

19. The method of claim 1, wherein each population of hydrogel beads comprises a single fluorophore.

20. The method of claim 1, wherein the first fluorophore and second fluorophore exhibit spectral overlap in their emission spectra.

21. The method of claim 1, wherein calculating a compensation or spectral unmixing matrix comprises determining the amount of spectral overlap between the first and second fluorophores.

22. The method of claim 6, comprising:
(vi) measuring fluorescence from the first and/or second fluorophore, and normalizing the measured fluorescence based on the compensation or spectral unmixing of step (iii).

23. A composition comprising:
(i) a first population of polymer beads comprising a first fluorophore; and
(ii) a second population of polymer beads comprising a second fluorophore;
wherein the first population of polymer beads and the second population of polymer beads each independently exhibit at least one optical property that is substantially similar to the optical property of a target cell.
